(19) United States
(12) Patent Application Publication
(10) Pub. No.: US 2022/0125959 A1
(43) Pub. Date: Apr. 28, 2022

(54) PROSTATE-SPECIFIC MEMBRANE ANTIGEN (PSMA) INHIBITORS AS DIAGNOSTIC AND RADIONUCLIDE THERAPEUTIC AGENTS

(71) Applicant: FIVE ELEVEN PHARMA INC., Philadelphia, PA (US)

(72) Inventors: Hank F. KUNG, Springfield, PA (US); Zhihao ZHA, Philadelphia, PA (US); Karl PLOESSL, Wilmington, DE (US); Seok Rye CHOI, Aston, PA (US)

(21) Appl. No.: 17/605,609

(22) PCT Filed: Apr. 27, 2020
(86) PCT No.: PCT/US2020/030085

§ 371 Date: Oct. 22, 2021

Related U.S. Application Data
(60) Provisional application No. 62/839,085, filed on Apr. 26, 2019.

(51) Int. Cl. A61K 51/04; A61P 35/00; A61K 47/54

(52) U.S. Cl. CPC A61K 51/0497; A61K 47/542; A61K 47/547; A61P 35/00

(57) ABSTRACT

The present disclosure relates to compounds according to Formula I. These compounds display very good binding affinities to the PSMA binding sites. They comprise a radioactive isotope or a chelating moiety that can be labeled with a radioactive metal such as [⁶⁸Ga]or [¹⁷⁷Lu]. The present disclosure also relates to pharmaceutical compositions comprising a pharmaceutical acceptable carrier and a compound of Formula I or a complex thereof, or a pharmaceutically acceptable salt thereof.

### FIELD OF THE INVENTION

This invention is in the field of radionuclide imaging and therapy agents. In particular, derivatives of urea-based prostate-specific membrane antigen (PSMA) inhibitors are disclosed, including derivatives with a chelating moiety are capable of chelating a radioactive metal, and derivatives with halogenated labeled phenyl.

### BACKGROUND OF THE INVENTION

Prostate-specific membrane antigen (PSMA) is a highly specific prostate epithelial cell membrane antigen. Its natural substrates are N-acetyl-aspartylglutamate and folyl-poly-γ-glutamates (prostate related PSMA) (Scheme 1).

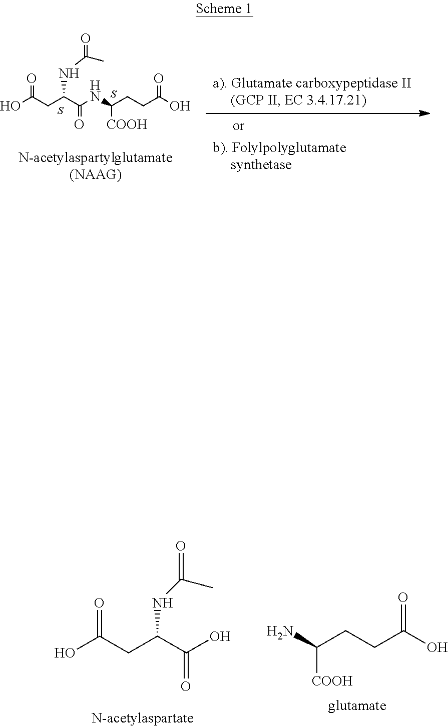

PSMA is highly expressed in various tumors, including prostate cancer. Often, PSMA expression increases in higher-grade cancers and metastatic diseases. In the vast majority of neovasculature in solid tumors, there is high expression of PSMA, but not in normal vasculature. This makes PSMA a suitable target for cancer detection and therapy.

A number of small molecule-based PSMA imaging agents have been reported in the literature. Different PSMA-targeting core structures have been employed, including: 2[(3-amino-3-carboxypropyl)(hydroxy)(phosphinyl)-methyl]pentane-1,5-dioic acid (GPI), 2-(3-mercaptopropyl)pentane-dioic acid (2-PMPA), phosphoramidates, and particularly, urea-Glu group (Glu-NH—CO—NH-Lys(Ahx)) (Scheme 2). See e.g. US2004054190; Kozikowski AP, et al., J. Med. Chem. 47:1729-38 (2004). Based on these binding core structures, many PSMA inhibitors were reported to be highly selective and potent. After labeling with different isotopes, they are disclosed as being useful in vivo imaging (SPECT or PET) as well as radionuclide therapy.

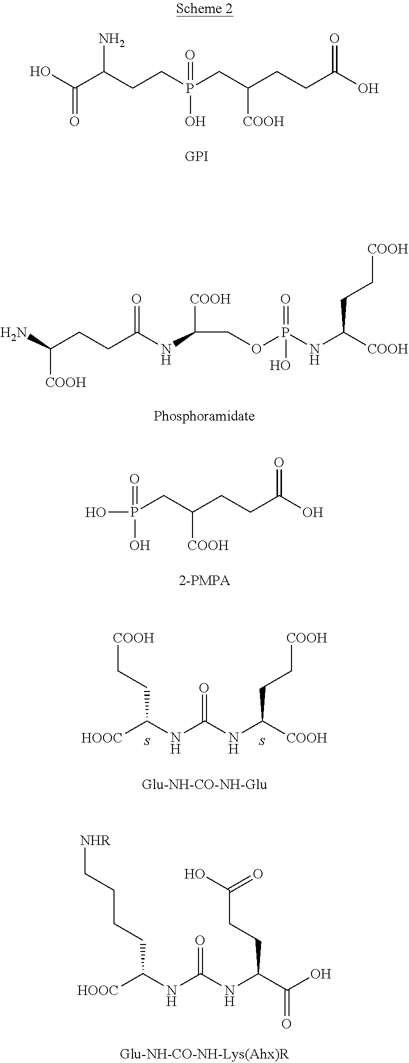

Several potential PSMA-targeted imaging agents using urea based ligand systems (Glu-NH—CO—NH or Glu-NH—CO—NH-Lys(Ahx)), including SPECT imaging agents: [¹²³I]MIP-1072, [¹²³I]MIP-1095, [^{99m}Tc]MIP-1404, and [^{99m}Tc]Tc-MIP-1405 (Scheme 3), have entered into clinical trials. Results of phase II clinical studies suggest that these SPECT PSMA imaging agents are suitable for the diagnosis of prostate and other related solid tumors.

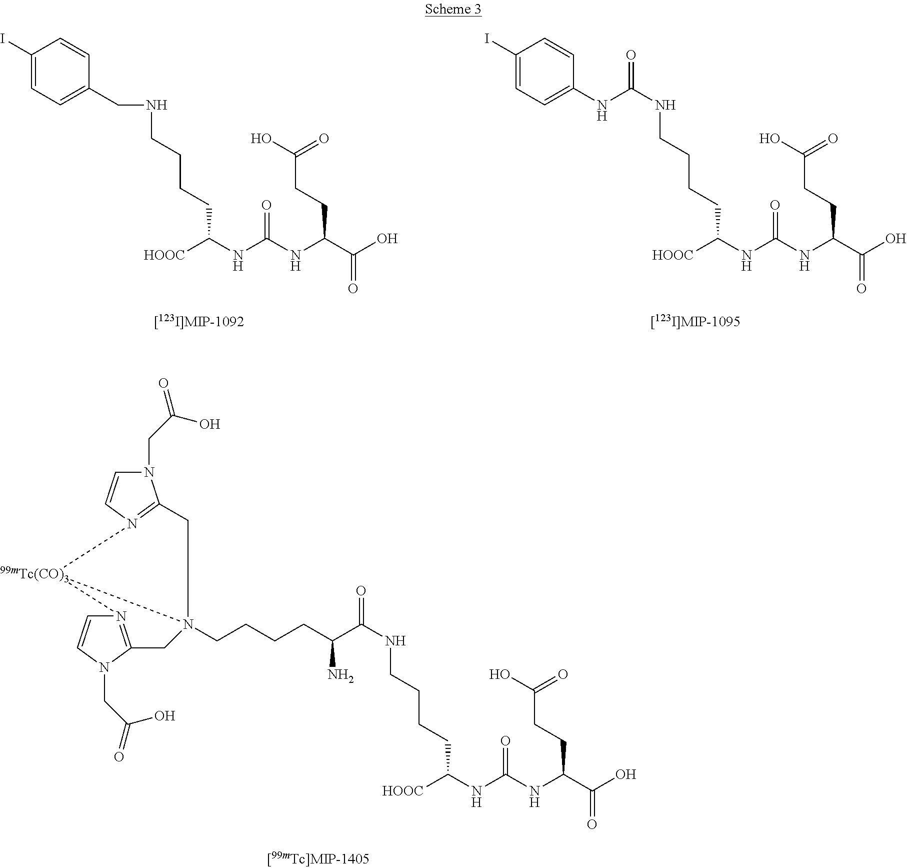

¹⁸F labeled PET imaging agents targeting PSMA have also been reported (Scheme 4).

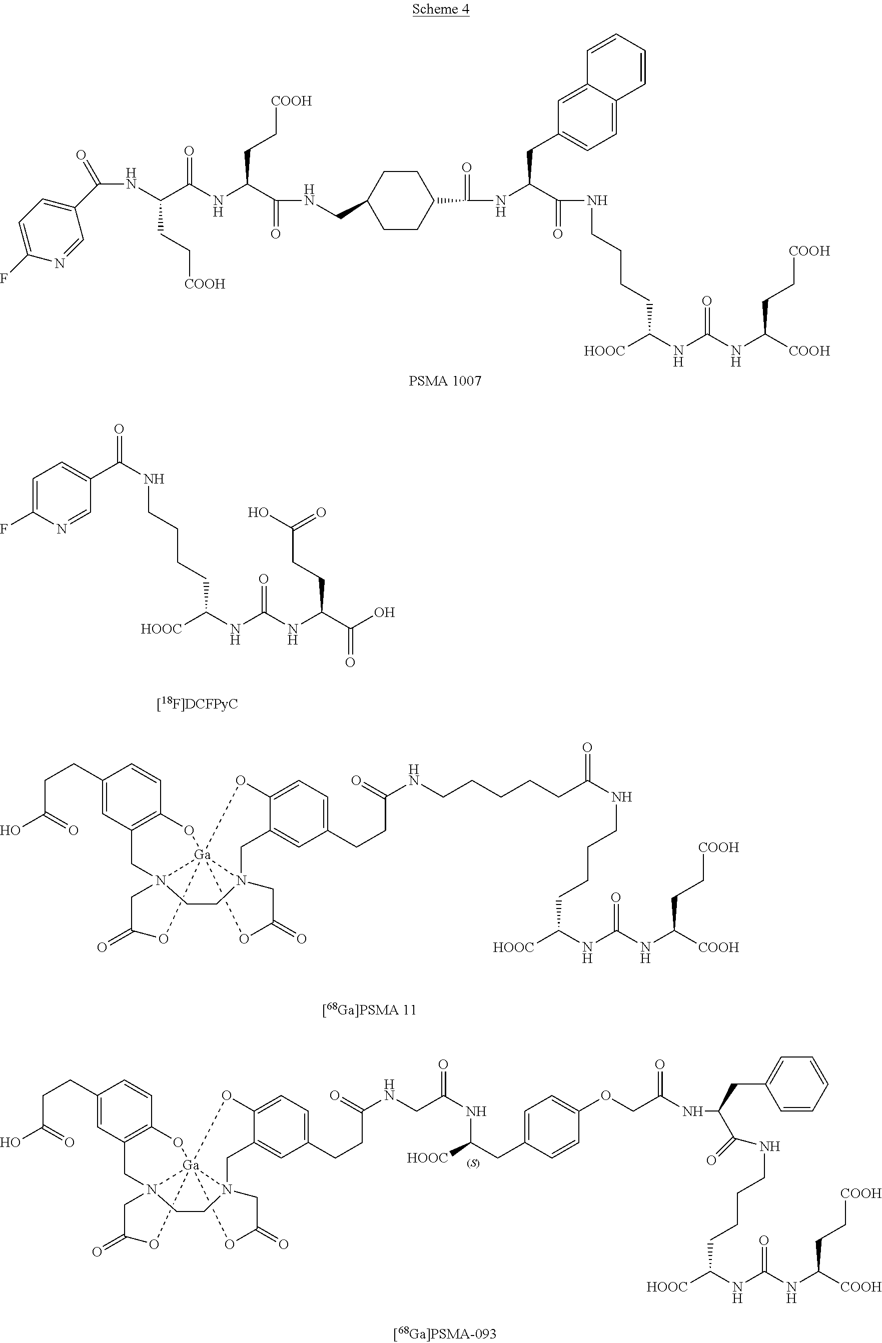

In the past two decades there are many reports on using ⁶⁸Ga labeled small molecules and peptides for imaging various tumors. Among them [⁶⁸Ga]DOTA-TOC, [⁶⁸Ga]DOTA-TATE, and [⁶⁸Ga]DOTA-NOC are employed as agents for the detection of neuroendocrine tumors (NET) expressing somatostatin receptors. ⁶⁸Ga labeled compound [⁶⁸Ga]PSMA-11is well studied (Scheme 4). Clinical data has been generated, which showed the ability to detect and monitor prostate cancer [4]. Additional ⁶⁸Ga labeled compounds targeting PSMA binding have been reported, including ⁶⁸Ga PSMA-093 (Scheme 4), which was reported to have improved tumor targeting properties and pharmacokinetics [5]. See U.S. Patent Application Publication No. 2016/0228587.

Based on targeting PSMA binding sites, which is over-expressed in majority of prostate cancer patients, ¹⁷⁷Lu labeled PSMA-617 and DOTAGA-(yl)-fk(sub-KuE) (PSMA-I&T) were reported as PSMA targeted radionuclide therapy (Scheme 5) (see Reviews [10-13] [14] [15]). Results of clinical trials for [¹⁷⁷Lu]PSMA 617 [16] and [¹⁷⁷Lu]PSMA I&T [17] (Scheme 5) were promising.

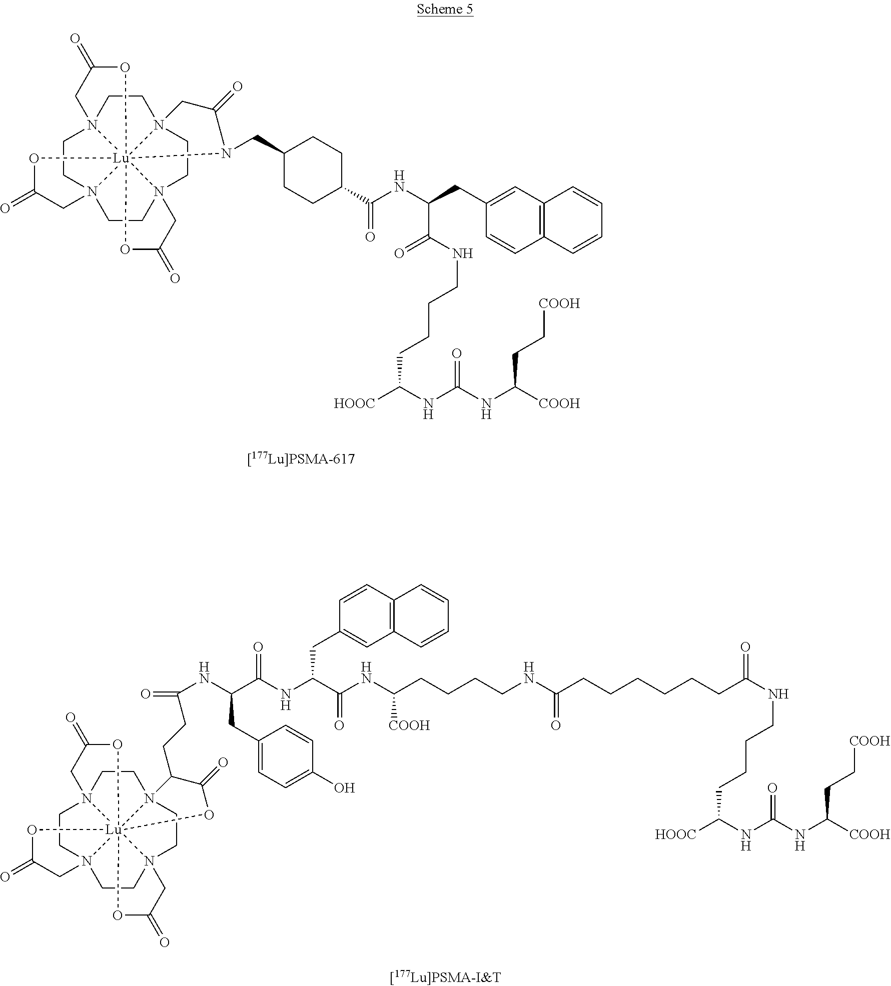

One other radionuclide for therapy is ¹³¹I. which emits electrons (beta radiation) with a physical half-life of 8.02 days and emitting maximal beta energy of 606 keV (89% abundance) and 364 keV gamma rays (81% abundance). There is a long history of using ¹³¹I iodide for treatment of thyroid cancer. This is a standard care of thyroid patients. It has been reported that ¹³¹I labeled MIP-1095 (Scheme 3) showed an high PSMA binding affinity (Ki=4.6 nM) and it is an attractive alternative PSMA targeting radionuclide therapeutic agent [1]. Previously, several radioactive iodinated imaging and therapeutic agents with structure modifications in the linker regions have been reported to have improved tumor targeting properties and pharmacokinetics. See U.S. Patent Application Publication No. 2016/0228587.

A need continued to exist to further improve the Glu-NH—CO—NH-Lys derivatives as PSMA inhibitor for in vivo imaging and radionuclide therapy.

### BRIEF SUMMARY OF THE INVENTION

In one embodiment, the present disclosure relates to a compound according to Formula I:

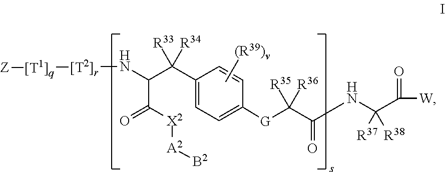

or a pharmaceutically acceptable salt thereof,
wherein

Z is a chelating moiety, or
a group having the structure of Z¹:

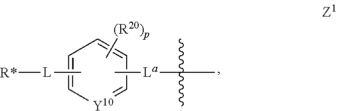

wherein Y¹⁰ is CH or N;
   each of L and L^{a} is independently a bond or a divalent linking moiety comprising 1 to 6 carbon atoms in a chain, a ring, or a combination thereof, wherein at least one carbon atom is optionally replaced with O, —NR³—, or —C(O)—;
   R* is a radioactive isotope;
   R²² is selected from the group consisting of alkyl, alkoxyl, halide, haloalkyl, and CN;
   p is an integer from 0 to 4, wherein when p is greater than 1, each
R²² is the same or different;

W is a PSMA-targeting ligand;

each T¹ independently has the structure of T¹¹ or T¹²:

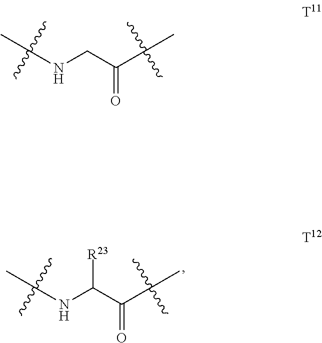

wherein R²³ is —(CH₂)ₐCO₂H, and a is an integer from 0 to 4;

each T² independently has the structure of of T²¹ or T²²:

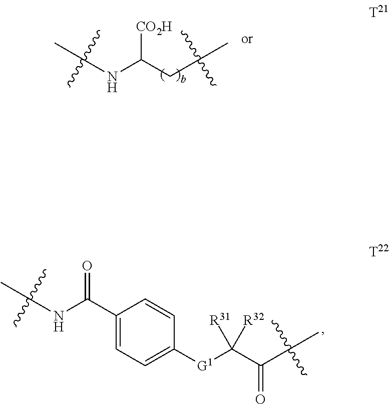

wherein b is an integer from 1 to 6, and G¹ is O, S, or NR³;

q is 0, 1, 2, or 3;

r is 0, 1, or 2;

A² is a bond or a divalent linking moiety comprising 1 to 20 carbon atoms in a chain, a ring, or a combination thereof, wherein one or more carbon atoms can be optionally replaced with O, —NR⁴⁰—, or —C(O)—;

B² is H,

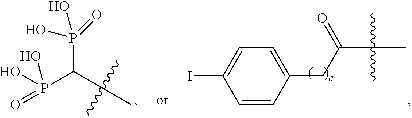

wherein c is an integer from 1 to 4,

G is O, S, or NR³;

X² is O, S, or —NR⁴¹—;

each of R³, R⁴⁰, and R⁴¹ is independently selected from the group consisting of hydrogen, alkyl, cycloalkyl, heterocycloalkyl, aryl, alkylaryl, and heteroaryl.

each of R³¹, R³², R³³, R³⁴, R³⁵, and R³⁶ is independently hydrogen, alkyl, alkoxyl, or halide;

each of R³⁷ and R³⁸ is independently hydrogen, alkyl, aryl, or alkylaryl;

each R³⁹ is independently selected from the group consisting of alkyl, alkoxyl, halide, haloalkyl, and CN;

s is 0 or 1; and

v is an integer from 0 to 4, wherein when v is greater than 1, each R³⁹ is the same or different;

provided that if s is 1, —X²-A²-B² is —OH, r is 0, q is 1, and T¹ is T¹¹,

then Z is not Z¹ or

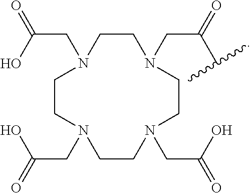 [SMILES: *C(=O)CN1CCN(CC(=O)O)CCN(CC(=O)O)CCN(CC(=O)O)CC1]

In one embodiment, the present disclosure relates to a method for imaging in a subject, comprising administering a radiolabeled compound disclosed herein to the subject; and obtaining an image of the subject or a portion of the subject. In another embodiment, the method for imaging comprises obtaining an image with a device that is capable of detecting positron emission.

Additionally, the disclosure relates to methods of making a compound of Formula I.

In another embodiment, the present disclosure relates to a method for treating one or more tumors in a subject, comprising administering an effective amount of the compound or complex disclosed herein to the subject. In some embodiments, the tumor is a PSMA-overexpressing tumor. In some embodiments, the tumor is prostate tumor, neuroendocrine tumor, or endocrine tumor. In some embodiments, the tumor is prostate tumor.

### BRIEF DESCRIPTION OF THE FIGURES

FIG. 1 shows HPLC chromatograms of radio-labeled [⁶⁸Ga]4. Stationary phase: Eclipse XDB-C18 column 5μ, 4.6×150 mm; Mobile phase: A: 0.1% TFA/water; B: 0.1% TFA/ACN; gradient: 0-8 min AB 100/0-0/100; 2 mL/min.

FIG. 2 shows HPLC chromatograms of radio-labeled [¹⁷⁷Lu]4. Stationary phase: Eclipse XDB-C18 column 5μ, 4.6×150 mm; Mobile phase: A: 0.1% TFA/water; B: 0.1% TFA/ACN; gradient: 0-4 min A/B 85/15-0/100, 4-11 min A/B 85/15 to 30/70, 11-14 min A/B 30/70 to 85/15; 1 mL/min.

FIG. 3 shows HPLC chromatograms of radiolabeled, protected intermediate [¹²⁵I]24, cold standard 26, and radioactive trace of final compound [¹²⁵I]26. Stationary phase: Agilent Porocell 120 EC-C18 column 2.7μ, 4.6×50 mm; Mobile phase: A: 0.1% TFA/water; B: 0.1% TFA/ACN; gradient: 0-1 min A/B 80/20, 1-16 min A/B 80/20 to 0/100, 16-16.5 min A/B 0/100 to 80/20, 16.5-20 min A/B 80/20; 2 mL/min.

### DETAILED DESCRIPTION OF THE INVENTION

Many different radionuclides and many different precision targets have been reported [8]. Theranostic approach provides a personalized approach for precision medicine. One of the suitable isotopes is Lu-177 [8, 18, 19]. Lutetium-177 (Lu-177) with a physical half-life of 6.65 days is a suitable therapeutic radionuclide, which emits Beta rays (490 keV), gamma rays, and X-rays (113 keV (3%), 210 keV (11%)).

Based on agents targeting PSMA, which is over-expressed in majority of prostate cancer patients, radiolabeled agents have been prepared for diagnostic imaging and radionuclide therapy. ¹⁷⁷Lu labeled PSMA-617 and DOTAGA-(yl)-fk(sub-KuE) (PSMA-I&T) were reported as PSMA targeted radionuclide therapy (see Reviews [10-13] [14] [15]. Results of clinical trials for PSMA-617 [16] and PSMA-I&T [17] as radionuclide therapeutic agents were very promising.

In the past two decades there are many reports on using radiometals labeled small molecules and peptides for imaging various tumors. Among them [⁶⁸Ga]DOTA-TOC, [⁶⁸Ga]DOTA-TATE, and [⁶⁸Ga]DOTA-NOC are commonly employed agents for the detection of neuroendocrine tumors (NET) expressing somatostatin receptors. Recently, [⁶⁸Ga]PSMA-11 has been reported as an effective PET imaging agent targeting over expression of PSMA in prostate cancer patients.

Additional chelates for making radionuclide therapeutic agents labeled with lutetium (Lu-177) have been reported. The chelating groups include many cyclic and acyclic polyaza carboxylic acids (Scheme 6) with stability constants (logK_{d}) between 15 to 30, respectively These improved chelates, 1,4,7,10-tetraazacyclodocecane,1-(glutaric acid)-4,7,10-triacetic acid (DOTAGA) and 1,4,7,10-tetraazacyclodocecane,1,7-(diglutaric acid)-4,10-diacetic acid (DOTA(GA)2), have the advantage of forming stable ¹⁷⁷Lu labeled complexes at room temperature (i.e. stable in vitro and in vivo), which simplifies preparation and makes it more suitable in a clinical setting.

Many compounds of the disclosure include DOTAGA and DOTA(GA)2, both of which can form stable chelating complexes with various radioactive metals (M), including ⁶⁸Ga (for diagnostic) [6] as well as ¹⁷⁷Lu (for radionuclide therapy) [7]. (Scheme 6).

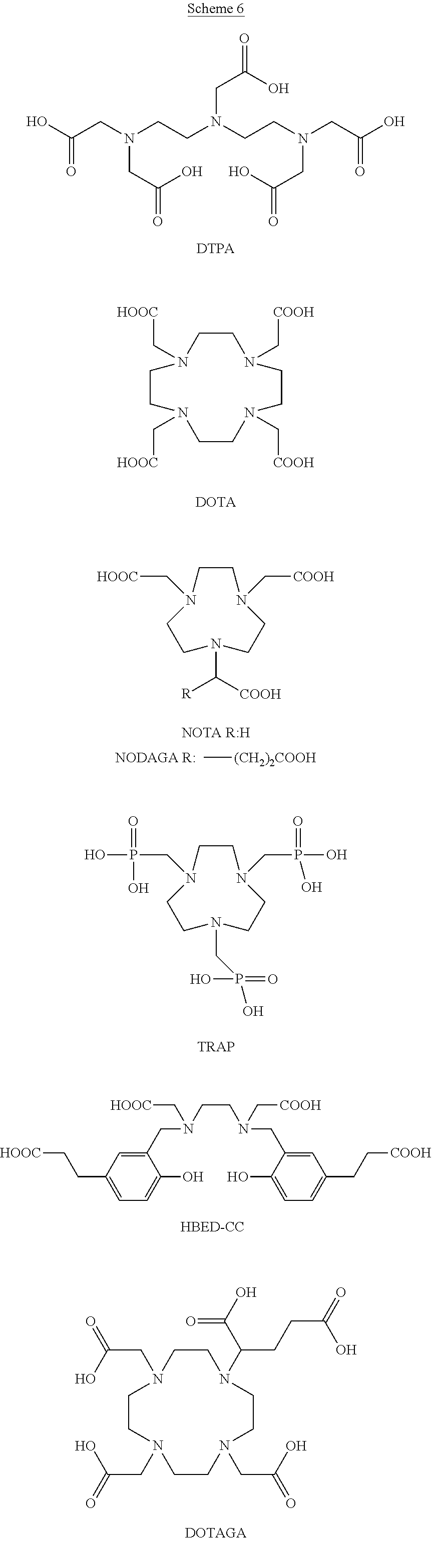

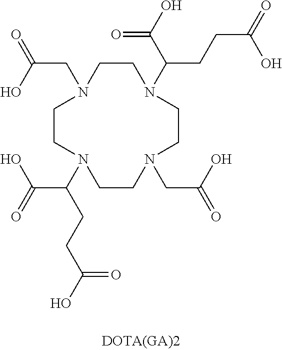 [SMILES: O=C(O)CCC(C(=O)O)N1CCN(CC(=O)O)CCN(C(CCC(=O)O)C(=O)O)CCN(CC(=O)O)CC1]

In the compounds or complexes disclosed herein, the in vivo biodistribution properties are improved by specific modification of the chemical structures (e.g., changing the linkers) of these compounds, for example, iodinated and lutetium labeled PSMA inhibitors. Structural adjustments have led to higher tumor uptake and faster renal excretion (reducing non-target radiation dose) in PSMA tumor bearing mice.

These new agents are valuable for radionuclide therapy, when labeled with beta or alpha-emitting isotopes; but these agents will also be useful as diagnostic agents when labeled with gamma-emitting isotopes.

Compounds with a novel phenoxy linker were reported. See U.S. Patent Application Publication No. 2017/0189568, which is incorporated herein by reference in its entirety. This series of PSMA inhibitors including the sub-structure of an urea based PSMA targeting moiety and a novel linker to different chelating groups had led to stable metal complexes (including Lu-177). They were tested by in vitro binding, tumor cell uptake as well as in vivo biodistribution studies. These PSMA inhibitors showed good binding affinity and in vivo targeting ability for prostate tumor bearing nude mice. For example, the novel PSMA inhibitors can have a chelating moiety, such as complexes or compounds A; or they can have a: radioactive metal DOTAGA complex, b: radioactive metal DOTA(GA)2 complex or c: radioactive halogen (Scheme 7).

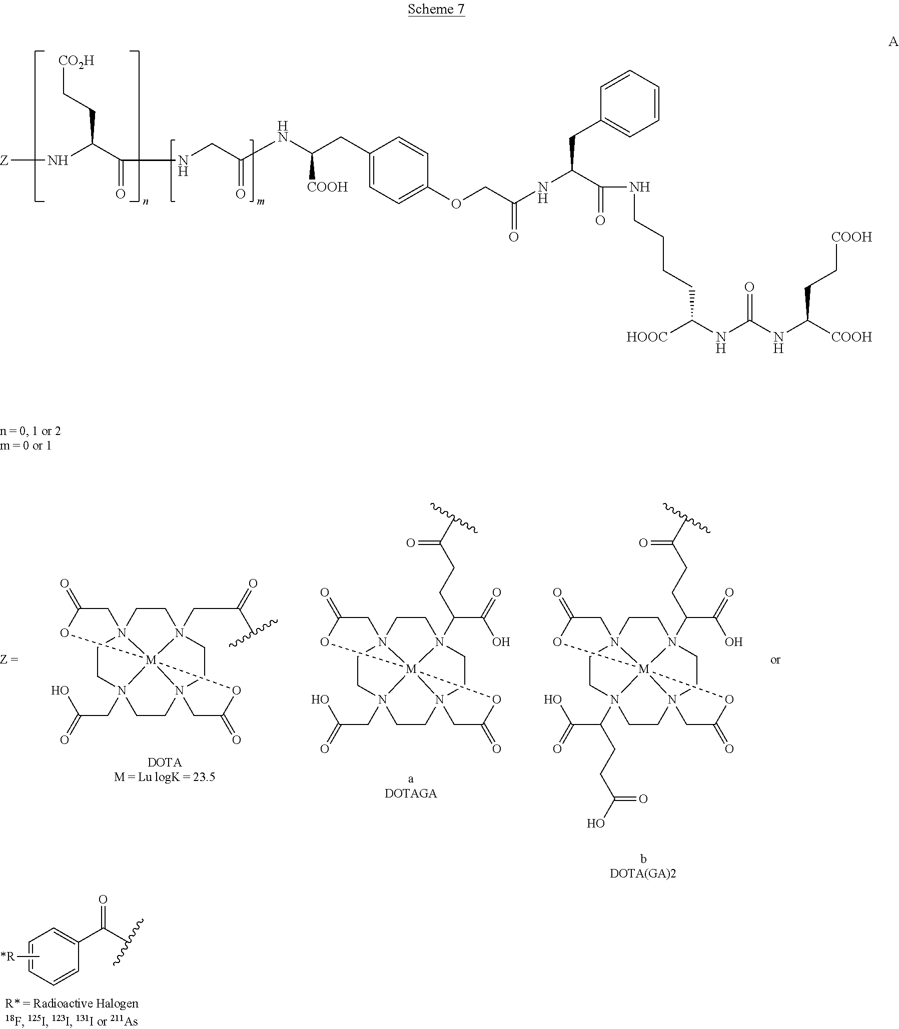

In one embodiment, the present disclosure relates to a compound according to Formula I:

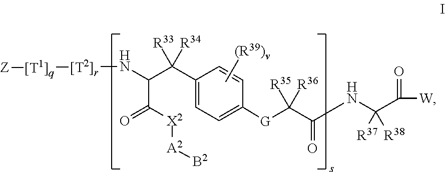

or a pharmaceutically acceptable salt thereof,
wherein

Z is a chelating moiety, or
a group having the structure of Z¹:

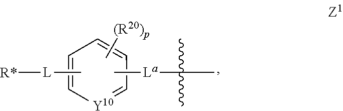

wherein Y¹⁰ is CH or N;
   each of L and L^{a} is independently a bond or a divalent linking moiety comprising 1 to 6 carbon atoms in a chain, a ring, or a combination thereof, wherein at least one carbon atom is optionally replaced with O, —NR³—, or —C(O)—;
   R* is a radioactive isotope;
   R²² is selected from the group consisting of alkyl, alkoxyl, halide, haloalkyl, and CN;
   p is an integer from 0 to 4, wherein when p is greater than 1, each R²² is the same or different;

W is a PSMA-targeting ligand;

each T¹ independently has the structure of T¹¹ or T¹²:

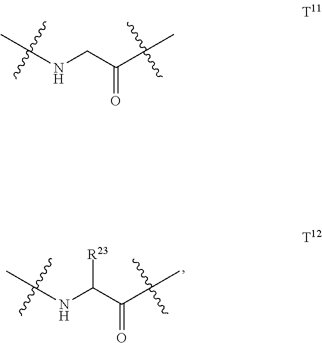

wherein R²³ is —(CH₂)ₐCO₂H, and a is an integer from 0 to 4;

each T² independently has the structure of of T²¹ or T²²:

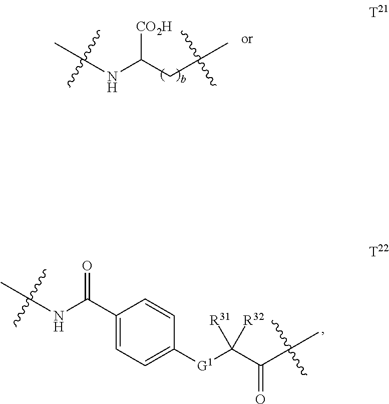

wherein b is an integer from 1 to 6, and G¹ is O, S, or NR³;

q is 0, 1, 2, or 3;

r is 0, 1, or 2;

A² is a bond or a divalent linking moiety comprising 1 to 20 carbon atoms in a chain, a ring, or a combination thereof, wherein one or more carbon atoms can be optionally replaced with O, —NR⁴⁰—, or —C(O)—;

B² is H,

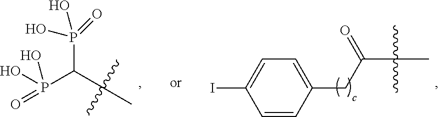

wherein c is an integer from 1 to 4,

G is O, S, or NR³;

X² is O, S, or —NR⁴¹—;

each of R³, R⁴⁰, and R⁴¹ is independently selected from the group consisting of hydrogen, alkyl, cycloalkyl, heterocycloalkyl, aryl, alkylaryl, and heteroaryl.

each of R³¹, R³², R³³, R³⁴, R³⁵, and R³⁶ is independently hydrogen, alkyl, alkoxyl, or halide;

each of R³⁷ and R³⁸ is independently hydrogen, alkyl, aryl, or alkylaryl;

each R³⁹ is independently selected from the group consisting of alkyl, alkoxyl, halide, haloalkyl, and CN;

s is 0 or 1; and

v is an integer from 0 to 4, wherein when v is greater than 1, each R³⁹ is the same or different;

provided that if s is 1, —X²-A²-B² is —OH, r is 0, q is 1, and T¹ is T¹¹,

then Z is not Z¹ or

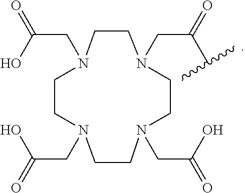 [SMILES: *C(=O)CN1CCN(CC(=O)O)CCN(CC(=O)O)CCN(CC(=O)O)CC1]

In some embodiments, Z is a chelating moiety. Chelating moieties are known in the art and they refer to metal-binding groups. In some embodiments, Z is a chelating moiety selected from the group consisting of DOTA, NOTA, NODAGA, DOTAGA, DOTA(GA)2, TRAP, NOPO, PCTA, DFO, DTPA, CHX-DTPA, AAZTA, DEDPA, and oxo-DO3A. These chelating moieties are derived from 1,4,7,10-tetraazacyclododecane-N,N′,N″,N′″-tetraacetic acid (DOTA), 1,4,7-triazacyclononane-1,4,7-triacetic acid (NOTA), 2-(4,7-bis(carboxymethyl)-1,4,7-triazonan-1-yl)pentanedioic acid (NODAGA), 1,4,7,10-tetraazacyclodocecane, 1-(glutaric acid)-4,7,10-triacetic acid (DOTAGA) and 1,4,7,10-tetraazacyclodocecane,1,7-(diglutaric acid)-4,10-diacetic acid (DOTA(GA)2), 1,4,7-triazacyclononane phosphinic acid (TRAP), 1,4,7-triazacyclononane-1-[methyl(2-carboxyethyl)phosphinic acid]-4,7-bis[methyl(2-hydroxymethyl)phosphinic acid](NOPO), 3,6,9,15-tetraazabicyclo[9.3.1.]pentadeca-1(15),11,13-triene-3,6,9-triacetic acid (PCTA), N′-{5-[Acetyl(hydroxy)amino]pentyl}-N-[5-({4-[(5-aminopentyl)(hydroxy)amino]-4-oxobutanoyl}amino)pentyl]-N-hydroxysuccinamide (DFO), Diethylenetriaminepentaacetic acid (DTPA), Trans-cyclohexyl-diethylenetriaminepentaacetic acid (CHX-DTPA), 1-oxa-4,7,10-triazacyclododecane-4,7,10-triacetic acid (oxo-Do3A), p-isothiocyanatobenzyl-DTPA (SCN-Bz-DTPA), 1-(p-isothiocyanatobenzyl)-3-methyl-DTPA (1B3M), 2-(p-isothiocyanatobenzyl)-4-methyl-DTPA (1M3B), 1-(2)-methyl-4-isocyanatobenzyl-DTPA (MX-DTPA). Useful chelating moieties are disclosed in US 2016/0228587, which is incorporated by reference herein in its entirety.

In some embodiments, Z is

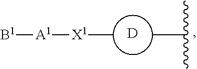

A¹ is a bond or a divalent linking moiety comprising 1 to 20 carbon atoms in a chain, a ring, or a combination thereof, wherein one or more carbon atoms can be optionally replaced with O, —NR⁴⁰—, or —C(O)—;

B¹ is H,

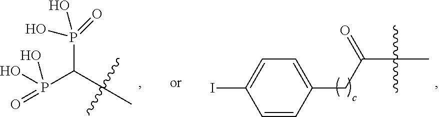

wherein c is an integer from 1 to 4;

X¹ is O, S, or —NR⁴¹—; and

D is a divalent chelating group derived from 1,4,7,10-tetraazacyclododecane-1,4,7,10-tetraacetic acid.

In some embodiments, D is selected from the group consisting of:

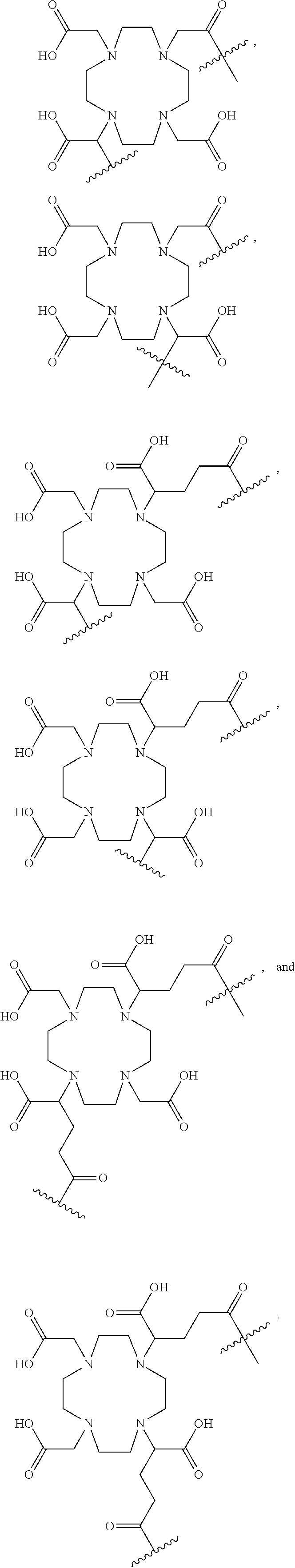

In these divalent chelating groups, the top right attachment site is connected to the T¹ group, and the bottom attachment site is connected to the X¹ group.

In some embodiments, D is selected from the group consisting of:

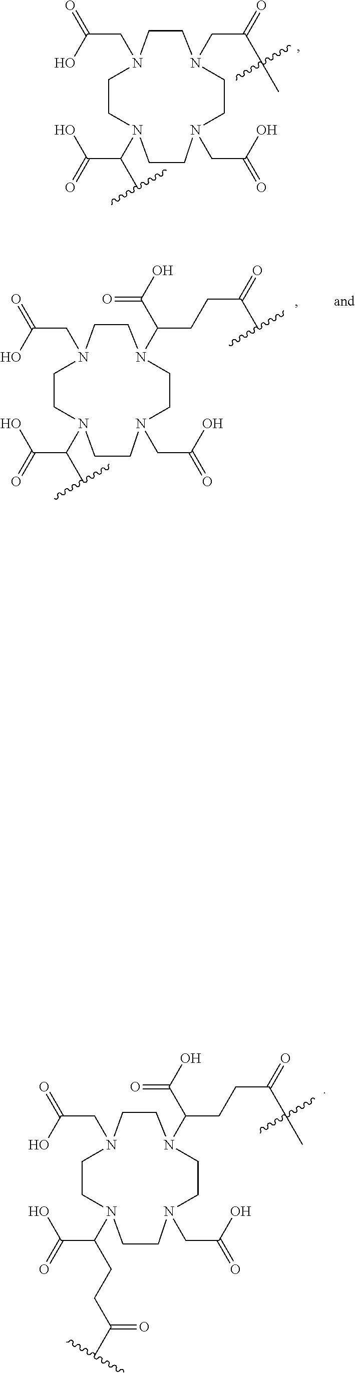 [SMILES: *C(=O)CN1CCN(CC(=O)O)CCN(C(*)C(=O)O)CCN(CC(=O)O)CC1 ; *C(=O)CCC(C(=O)O)N1CCN(CC(=O)O)CCN(C(*)C(=O)O)CCN(CC(=O)O)CC1 ; *C(=O)CCC(C(=O)O)N1CCN(CC(=O)O)CCN(C(CCC(*)=O)C(=O)O)CCN(CC(=O)O)CC1]

In some embodiments, D is selected from the group consisting of:

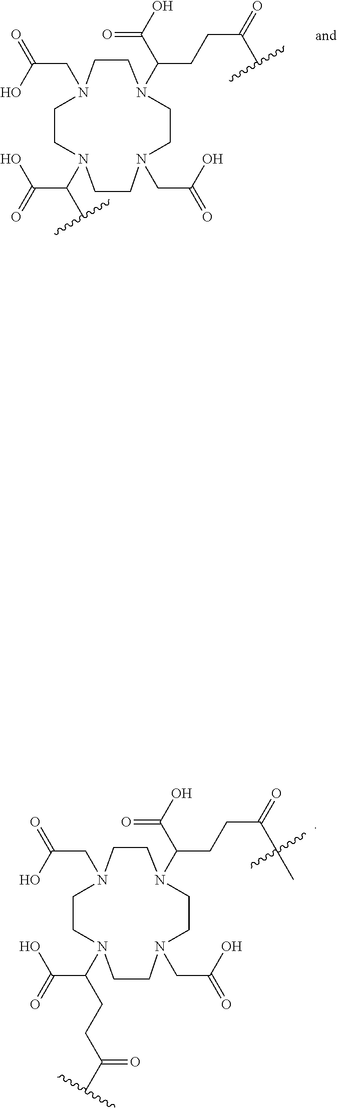 [SMILES: *C(=O)CCC(C(=O)O)N1CCN(CC(=O)O)CCN(C(*)C(=O)O)CCN(CC(=O)O)CC1 ; *C(=O)CCC(C(=O)O)N1CCN(CC(=O)O)CCN(C(CCC(*)=O)C(=O)O)CCN(CC(=O)O)CC1]

In some embodiments, A¹ is a bond or a divalent linking moiety comprising 1 to 16 carbon atoms in a chain, a ring, or a combination thereof, wherein one or more carbon atoms can be optionally replaced with O, —NR⁴⁰—or —C(O)—. In some embodiments, A¹ is a bond or —(CH₂)ₙ—, —(CH₂)ₙC(O)NH—, —(CH₂CH₂O)ₙ—, or —(CH₂CH₂O)ₙ(CH₂CH₂NH)ₙ—; and

each n is independently 1, 2, 3, or 4. In some embodiments, A¹ is a bond, —(CH₂)ₙC(O)NH—, or —(CH₂CH₂O)ₙ(CH₂CH₂NH)ₙ—; and n is 1, 2, or 3. In some embodiments, A¹ is a bond, —(CH₂)C(O)NH—, or —(CH₂CH₂O)₂(CH₂CH₂NH)—.

In some embodiments, B² is H,

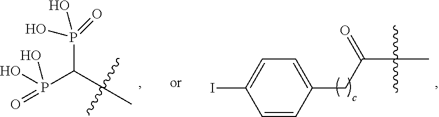

wherein c is an integer from 1 to 3. In some embodiments, c is 3.

In some embodiments, X¹ is O or —NH—. In some embodiments, X¹ is O, A¹ is a bond, and B¹ is H. In some embodiments, X¹ is —NH—, A¹ is —(CH₂)C(O)NH— or —(CH₂CH₂O)₂(CH₂CH₂NH)—, and B¹ is

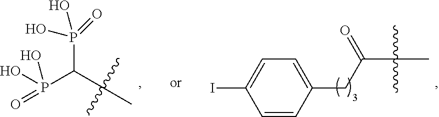

In some embodiments, Z is selected from the group consisting of:

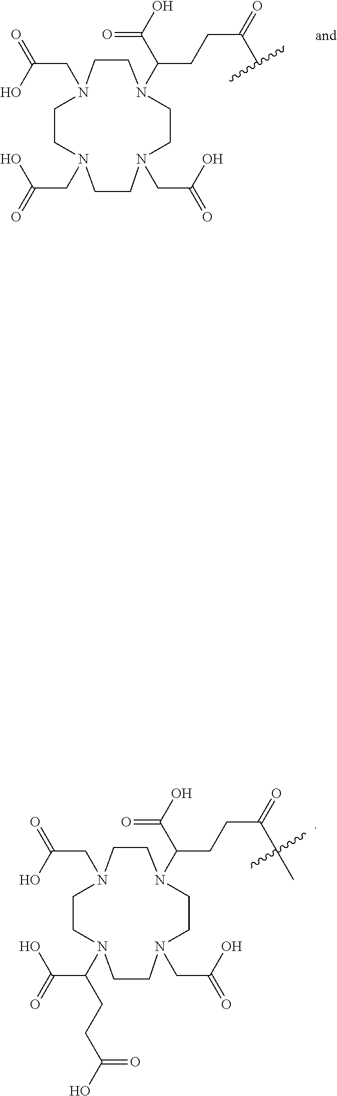 [SMILES: *C(=O)CCC(C(=O)O)N1CCN(CC(=O)O)CCN(CC(=O)O)CCN(CC(=O)O)CC1 ; *C(=O)CCC(C(=O)O)N1CCN(CC(=O)O)CCN(C(CCC(=O)O)C(=O)O)CCN(CC(=O)O)CC1]

In some embodiments, Z is selected from the group consisting of:

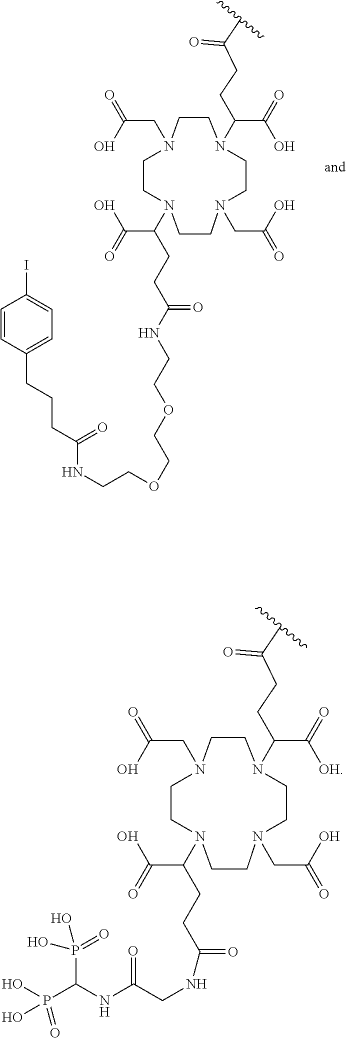

In some embodiments, Z is a group having the structure of Z¹:

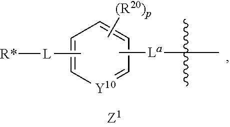

wherein Y¹⁰ is CH or N;
each of L and L^{a} is independently a bond or a divalent linking moiety comprising 1 to 6 carbon atoms in a chain, a ring, or a combination thereof, wherein at least one carbon atom is optionally replaced with O, —NR³—, or —C(O)—;
R* is a radioactive isotope;
R²² is selected from the group consisting of alkyl, alkoxyl, halide, haloalkyl, and CN;
p is an integer from 0 to 4, wherein when p is greater than 1, each R²² is the same or different.

Useful radioactive isotopes (i.e., radioisotopes) include positron emitting and photon emitting isotopes. Radioactive isotopes are known in the art, and they can be, for example, ¹¹C, ¹⁸F, ¹²³I, ¹²⁴I, ¹²⁵I, ¹³¹I, and ²¹¹As. ¹²⁴I can be used for PET imaging. ²¹¹As can be used for radionuclide therapy. In some embodiments, the radioactive isotopes are radioactive halogens. In some embodiments, the radioactive isotopes are photon emitting and can be used in SPECT, such as ¹²³I and ¹³¹I.

In some embodiments, L is a bond or a divalent linking moiety comprising 1 to 6 carbon atoms in a chain, a ring, or a combination thereof, wherein at least one carbon atom is optionally replaced with O, —NR³—, or —C(O)—. In some embodiment, L is a bond. In another embodiment, L is a divalent linking moiety comprising a C₁-C₆ alkylene group wherein at least one carbon atom is optionally replaced with O, —NR³—, or —C(O)—. In some embodiments, L is (CH₂)ₙ, —(OCH₂CH₂)ₙ—, —(NHCH₂CH₂)ₙ—, or —C(O)(CH₂)ₙ—, wherein n is 1, 2, or 3. In another embodiment, L is —OCH₂CH₂—. Other seful examples of the divalent linking moiety include —CH₂—, —CH₂CH₂—, —CH₂CH₂CH₂—, —OCH₂CH₂CH₂—, —NHCH₂CH₂—, —NHCH₂CH₂CH₂—, —COCH₂—, —COCH₂CH₂—, and —COCH₂CH₂CH₂—.

In some embodiments, L^{a} is a bond or a divalent linking moiety comprising 1 to 6 carbon atoms in a chain, a ring, or a combination thereof, wherein at least one carbon atom is optionally replaced with O, —NR³—, or —C(O)—. In another embodiment, L^{a} is a divalent linking moiety comprising a C₁-C₆ alkylene group wherein at least one carbon atom is optionally replaced with O, —NR³—, or —C(O)—. In some embodiments, L^{a} is —C(O)—.

In some embodiments, R²² is selected from the group consisting of C₁-C₄ alkyl, C₁-C₄ alkoxyl, halide, halo C₁-C₄ alkyl, and CN. In some embodiments, p is 0, 1, or 2. In some embodiments, p is 0.

In some embodiments, Y¹⁰ is CH. In some embodiments, Y¹⁰ is N.

In some embodiments, Z has the structure:

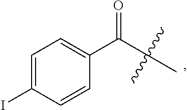 [SMILES: *C(=O)c1ccc(I)cc1]

wherein I (iodine) is radioactive. In some embodiments, the radioactive iodine is ¹²⁵I. In some embodiments, the radioactive iodine is ¹³¹I.

PSMA-targeting ligands are known in the art and they refer to groups that can bind to PSMA. PSMA-targeting ligands can be urea-based ligand systems discussed herein.

In some embodiments, the PSMA-targeting ligand W has the structure:

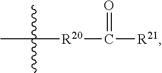

wherein R²⁰ and R²¹ are each independently an amino acid residue linked via an amino group thereof to the adjacent —C(O)— group.

In some embodiments, W has the structure:

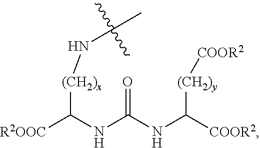

wherein R² is hydrogen or a carboxylic acid protecting group, x is an integer from 1 to 6, and y is an integer from 1 to 4. In one embodiment, W has the structure:

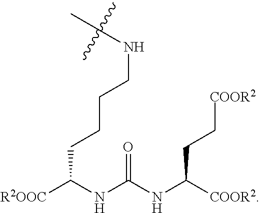

In certain embodiments, the compounds of the present disclosure are represented by generalized Formula I, and the attendant definitions.

The moiety -[T¹]_{q}-[T²]ᵣ- represents a linking moiety. In some embodiments, each T¹ independently has the structure of T¹¹ or T¹²:

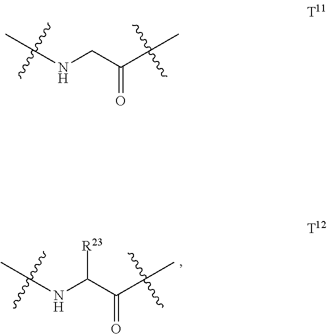

wherein R²³ is —(CH₂)ₐCO₂H, a is an integer from 0 to 4. In some embodiments, a is 0, 1, or 2. In some embodiments, a is 2.

In some embodiments, T¹² is:

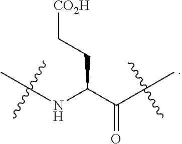 [SMILES: *N[C@@H](CCC(=O)O)C(*)=O]

In some embodiments, -[T¹]_{q}- is:

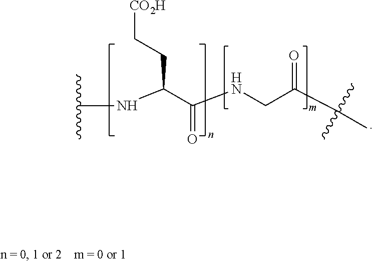

In some embodiments, each T² independently has the structure of of T²¹ or T²²:

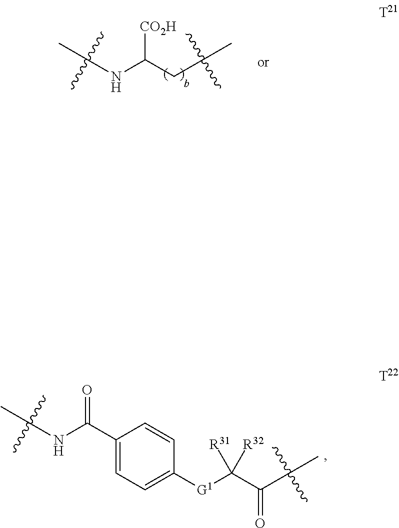

wherein b is an integer from 1 to 6, and G¹ is O, S, or NR³. In some embodiments, b is 1, 2, 3, or 4. In some embodiments, b is 3 or 4. In some embodiments, G¹ is O or —NH—. In some embodiments, G¹ is O. In some embodiments, each of R³¹ and R³² is independently hydrogen, C₁-C₄ alkyl, C₁-C₄ alkoxyl, or halide. In some embodiments, both R³¹ and R³² are hydrogen.

In some embodiments, -[T²]ᵣ- is:

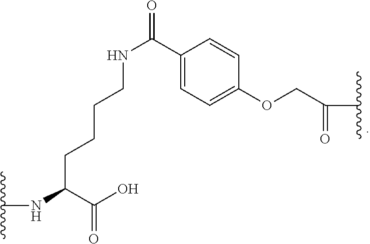 [SMILES: *N[C@@H](CCCCNC(=O)c1ccc(OCC(*)=O)cc1)C(=O)O]

In some embodiments, A² is a bond or a divalent linking moiety comprising 1 to 16 carbon atoms in a chain, a ring, or a combination thereof, wherein one or more carbon atoms can be optionally replaced with O, or —C(O)—. In some embodiments, A² is a bond or —(CH₂)ₙ—, —(CH₂)ₙC(O)O—, —(CH₂)ₙC(O)NH—, —(CH₂CH₂O)ₙ—, or —(CH₂CH₂O)ₙ(CH₂CH₂NH)ₙ—; and each n is independently 1, 2, 3, or 4. In some embodiments, A² is a bond or —(CH₂)ₙC(O)NH—; and n is 1, 2, or 3. In some embodiments, A² is a bond or —(CH₂)C(O)NH—.

In some embodiments, B² is H,

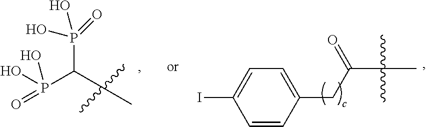

wherein c is an integer from 1 to 3. In some embodiments, c is 3.

In some embodiments, X² is O or —NH—. In some embodiments, X² is O, A² is a bond, and B² is H. In some embodiments, X² is —NH—, A² is a bond or —(CH₂)C(O)NH—, and B² is

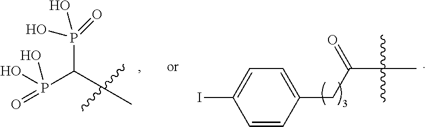

In some embodiments, each of R³, R⁴⁰, and R⁴¹ is independently selected from the group consisting of hydrogen, C₁-C₄ alkyl, C₁-C₆ cycloalkyl, heterocycloalkyl, aryl, C₁-C₄ alkylaryl, and heteroaryl. In some embodiments, each of R³, R⁴⁰, and R⁴¹ is hydrogen.

In some embodiments, each of R″, R³⁴, R³⁵, and R³⁶ is independently hydrogen, C₁-C₄ alkyl, C₁-C₄ alkoxyl, or halide. In some embodiments, R³³, R³⁴, R³⁵, and R³⁶ are hydrogen.

In some embodiments, each of R³⁷ and R³⁸ is independently hydrogen, C₁-C₄ alkyl, aryl, or C₁-C₄ alkylaryl. In some embodiments, each of R³⁷ and R³⁸ is independently hydrogen, phenyl, benzyl, or methylnaphthyl.

In some embodiments, each R³⁹ is independently selected from the group consisting of C₁-C₄ alkyl, C₁-C₄ alkoxyl, halide, halo C₁-C₄ alkyl, and CN. In some embodiments, each R³⁹ is independently methyl, methoxyl, halomethyl, or halide. In some embodiments, v is 0, 1, or 2. In some embodiments, v is 0.

In some embodiments, the compounds of Formula I have the structure of Formula I-A:

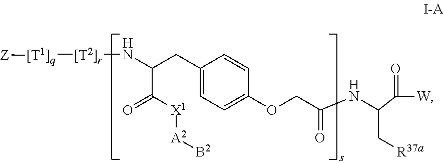

or a pharmaceutically acceptable salt thereof, wherein R^{37a} is optionally substituted phenyl or optionally substituted naphthyl.

In some embodiments, the compounds of Formula I have the structure of Formula I-B:

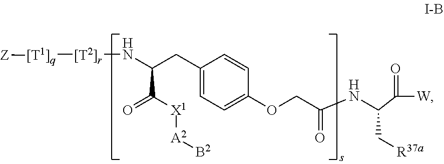

or a pharmaceutically acceptable salt thereof, wherein R^{37a} is optionally substituted phenyl or optionally substituted naphthyl.

In some embodiments, the compounds of Formula I have the structure of the following formulae:

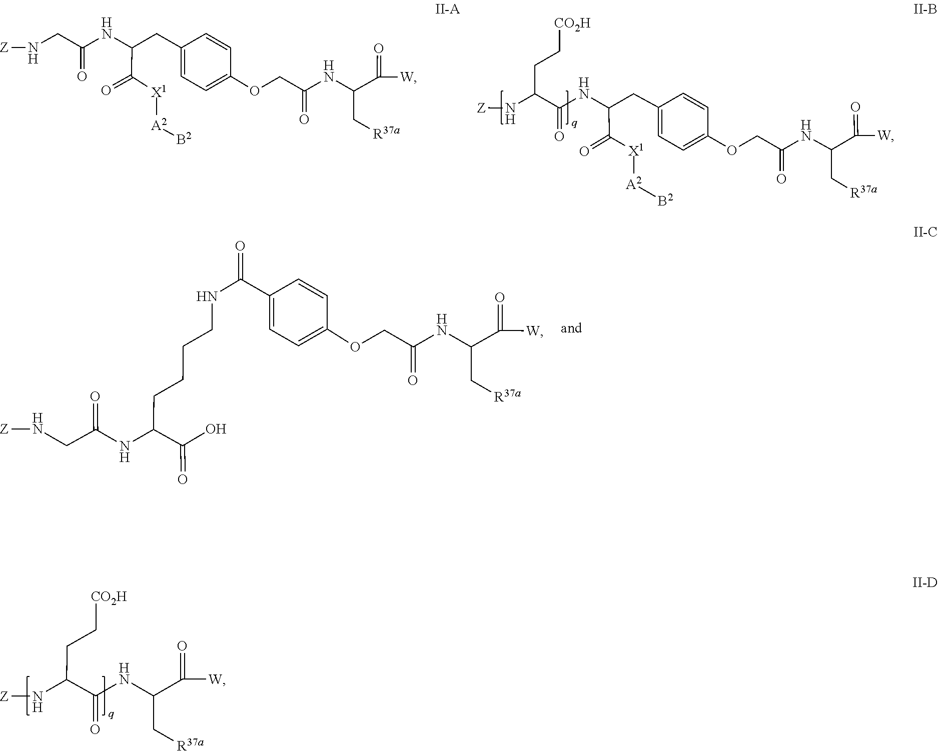

or a pharmaceutically acceptable salt thereof, wherein q is 1 or 2.

In some embodiments, the compounds of Formula I have the structure of the following formulae:

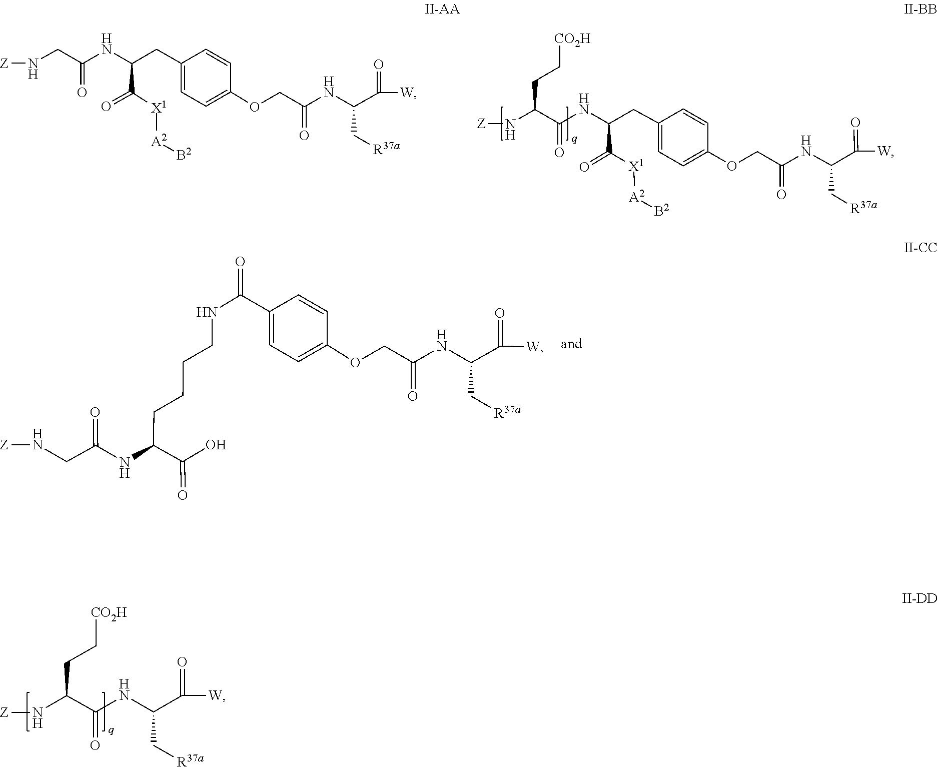

or a pharmaceutically acceptable salt thereof, wherein q is 1 or 2.

In some embodiments, the compounds of Formula I have the structure of Formula III-A:

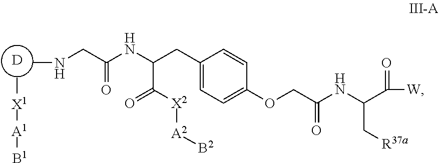

or a pharmaceutically acceptable salt thereof.

In some embodiments, the compounds of Formula I have the structure of Formula III-B:

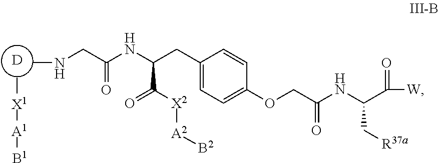

or a pharmaceutically acceptable salt thereof.

In some embodiments, the compounds of Formula I have the structure of Formula IV-A or IV-B:

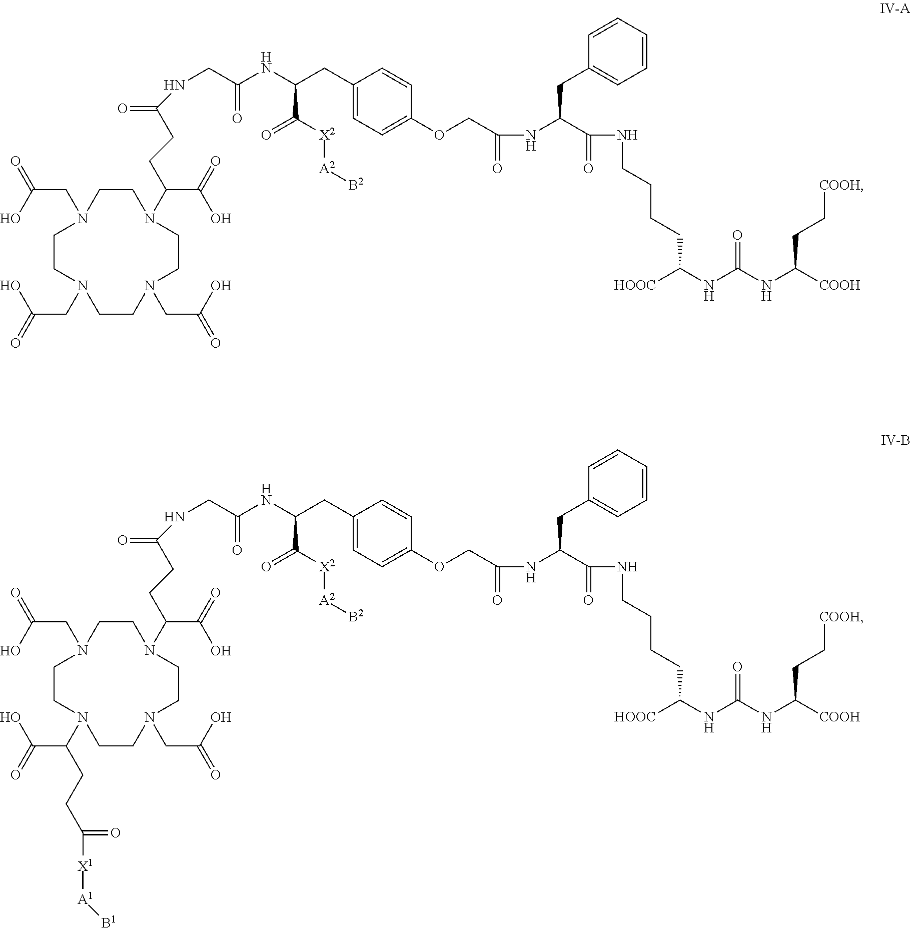

or a pharmaceutically acceptable salt thereof.

In some embodiments, R^{37a} is an aryl. In one embodiment, R^{37a} is optionally substituted phenyl. In another embodiment, R^{37a} is optionally substituted naphthyl. In some embodiments, R^{37a} is phenyl.

The definitions of A¹ , B¹, X¹, A², B², X², T¹, T², q, r, Z, and W described above for Formula I apply to any of Formulae I-A, I-B, II-A, II-B, II-C, II-D, II-AA, II-BB, II-CC, II-DD, III-A, III-B, IV-A, and IV-B.

In some embodiments, the compounds of Formula I have the following structures:

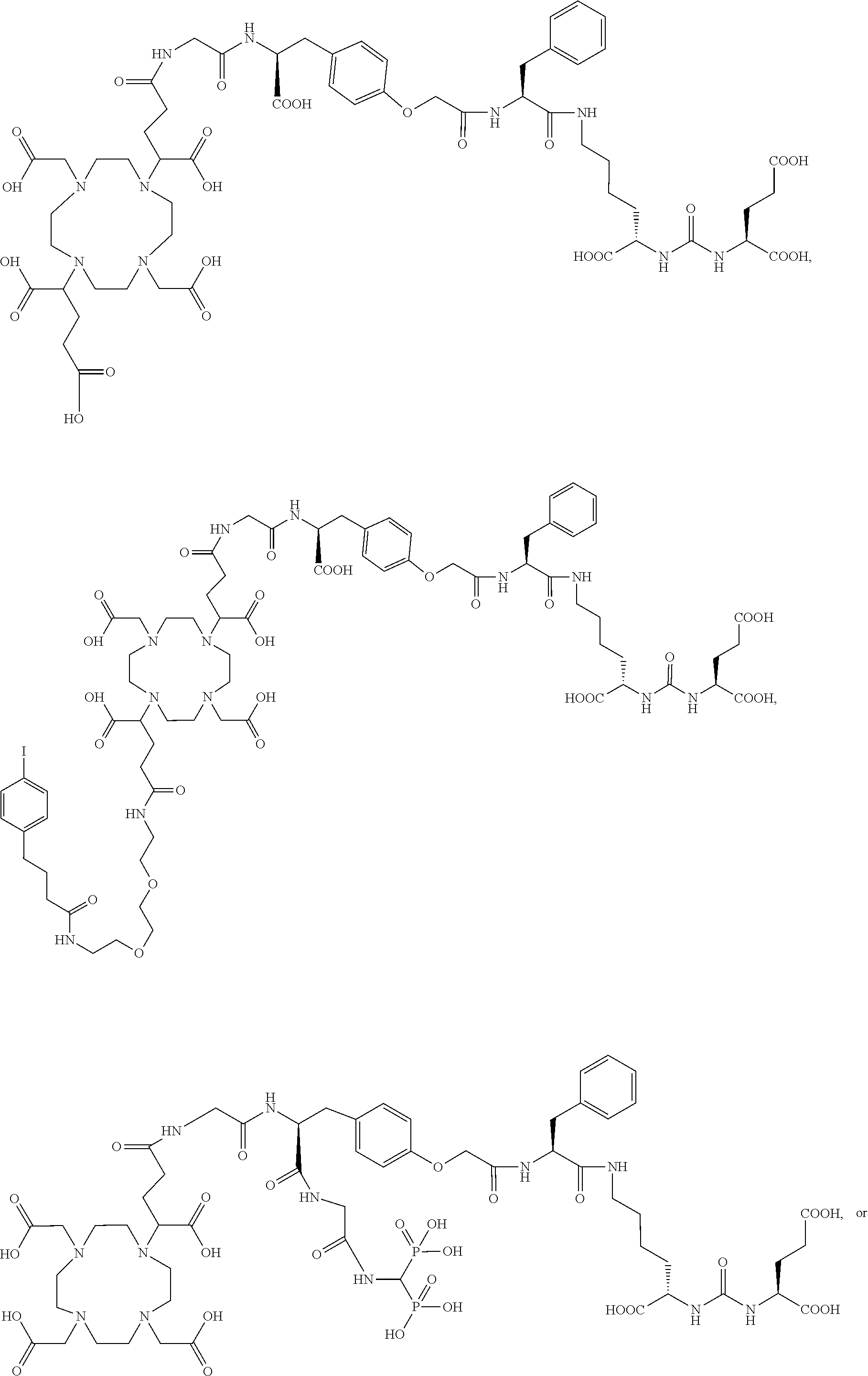

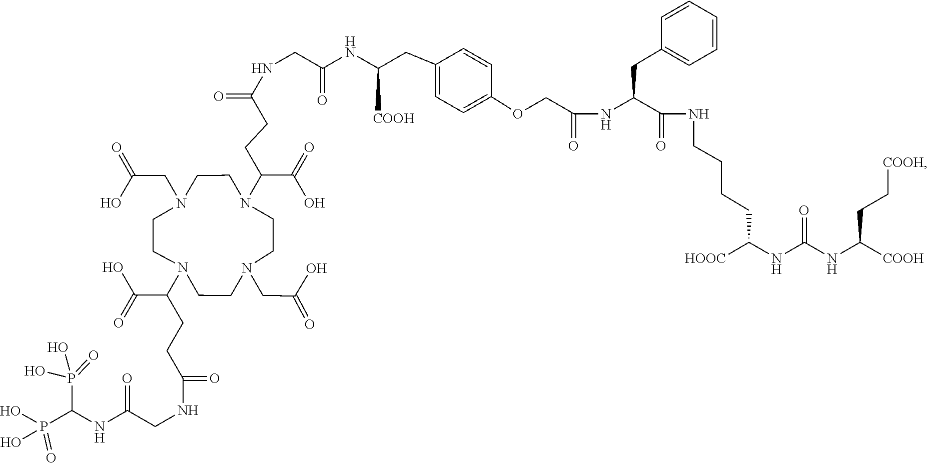

or a pharmaceutically acceptable salt thereof.

In some embodiments, the compounds of Formula I have the following structures:

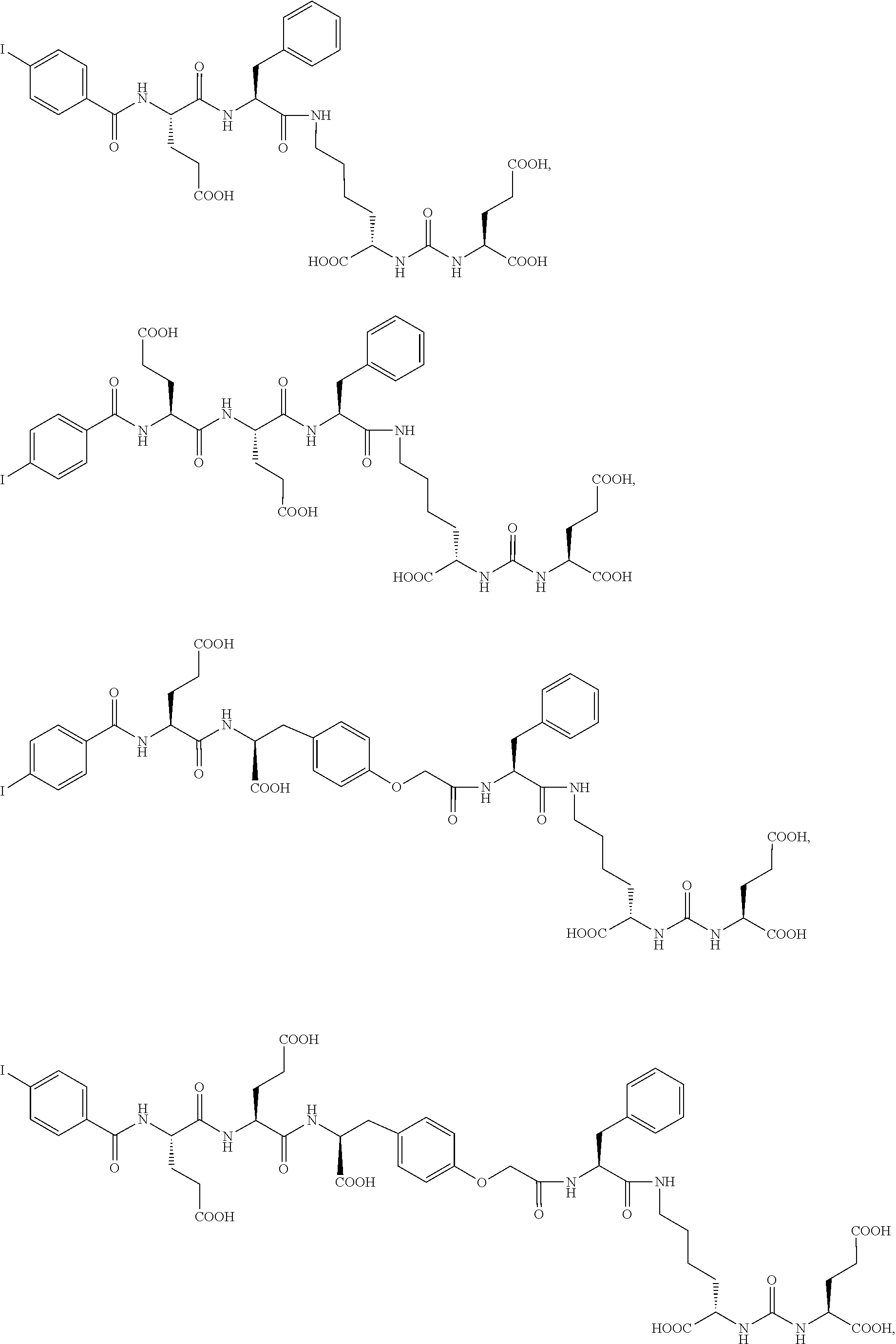

or a pharmaceutically acceptable salt thereof, wherein I (iodine) is radioactive. In some embodiments, the radioactive iodine is ¹²⁵I. In some embodiments, the radioactive iodine is ¹³¹I.

In some embodiments, the present disclosure relates to a complex comprising a compound according to Formula I disclosed herein chelated to a metal M wherein Z is a chelating moiety. In some embodiments, the metal M is selected from the group consisting of ²²⁵Ac, ⁴⁴Sc, ⁴⁷Sc, ^{203/212}Pb, ⁶⁷Ga, ⁶⁸Ga, ⁷²As, ^{99m}Tc, ¹¹¹In, ⁹⁰Y, ⁹⁷Ru, ⁶²Cu, ⁶⁴Cu, ⁵²Fe, ^{52m}Mn, ¹⁴⁰La, ¹⁷⁵Yb, ¹⁵³Sm, ¹⁶⁶Ho, ¹⁴⁹Pm, ¹⁷⁷Lu, ¹⁴²Pr, ¹⁵⁹Gd, ²¹³Bi, ⁶⁷Cu, ¹¹¹Ag, ¹⁹⁹Au, ¹⁶¹Tb, and ⁵¹ Cr. In some embodiments, the metal M is ⁶⁸Ga or ¹⁷⁷Lu. In some embodiments, the metal M is ⁶⁸Ga. In some embodiments, the metal M is ¹⁷⁷Lu.

An attractive and versatile approach in obtaining radiopharmaceuticals for PET/CT is the use of a ⁶⁸Ge/⁶⁸Ga generator to produce ⁶⁸Ga (T_{1/2}=68 min) PET imaging agents. There are several advantages for using ⁶⁸Ga for PET imaging: (1) It is a short-lived positron emitter (half-life 68 min, β⁺). (2) A ⁶⁸Ge/⁶⁸Ga generator readily produces ⁶⁸Ga in a laboratory setting without a nearby cyclotron. (3) The parent, ⁶⁸Ge, has a physical half-life of 270 days, providing a useful life of 6 to 12 months. (4) There are several commercial vendors now supplying this generator for clinical practice on a routine basis. (5) The coordination chemistry for Ga(III) is highly flexible and large number of Ga chelates with varying stability constants and metal chelating selectivity have been reported; It has been demonstrated that ⁶⁸Ga radiopharmaceuticals target various tissues or physiological processes for cancer diagnosis.

In some embodiments, the complex has the structure:

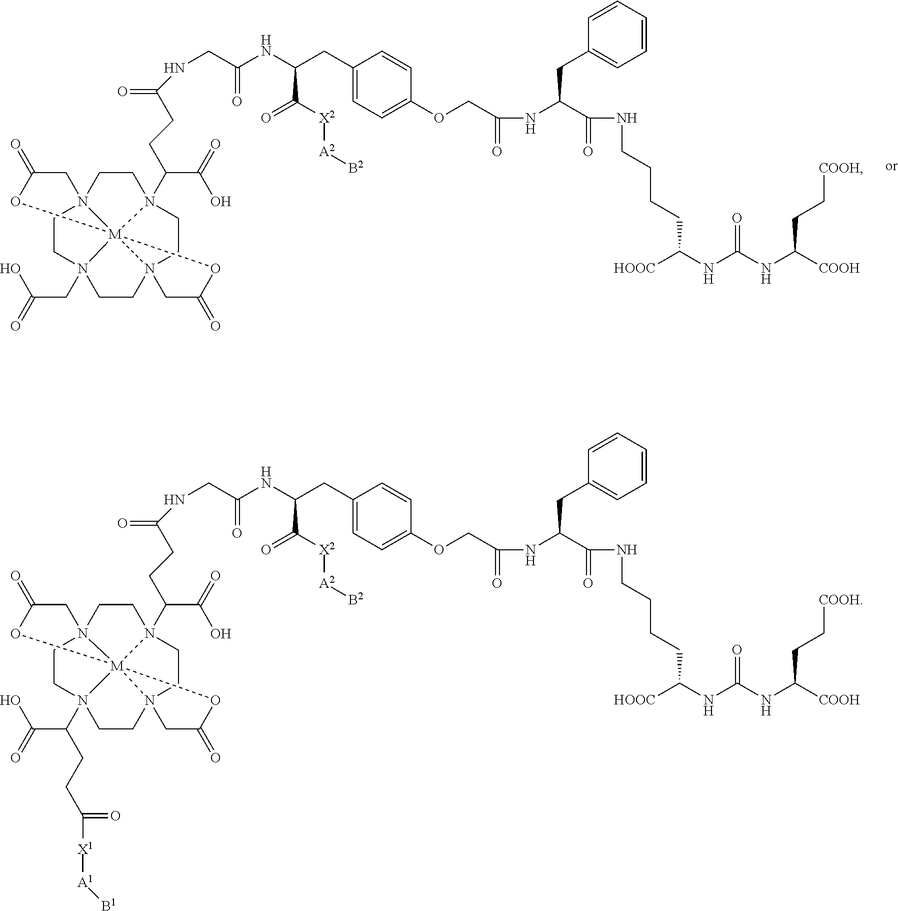

or a pharmaceutically acceptable salt thereof, wherein X¹, X², A¹, A², B¹, B², and M are defined herein. In some embodiments, X¹ is O or —NH—; X² is O or —NH—; A¹ is a bond, —(CH₂)C(O)NH—, or —(CH₂CH₂O)₂(CH₂CH₂NH)—; A² is a bond or —(CH₂)C(O)NH—; and each of B¹ and B² is independently H,

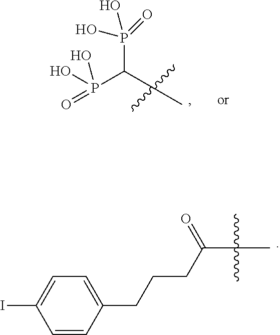 [SMILES: *C(P(=O)(O)O)P(=O)(O)O ; *C(=O)CCCc1ccc(I)cc1]

In some embodiments, the complex has the structure:

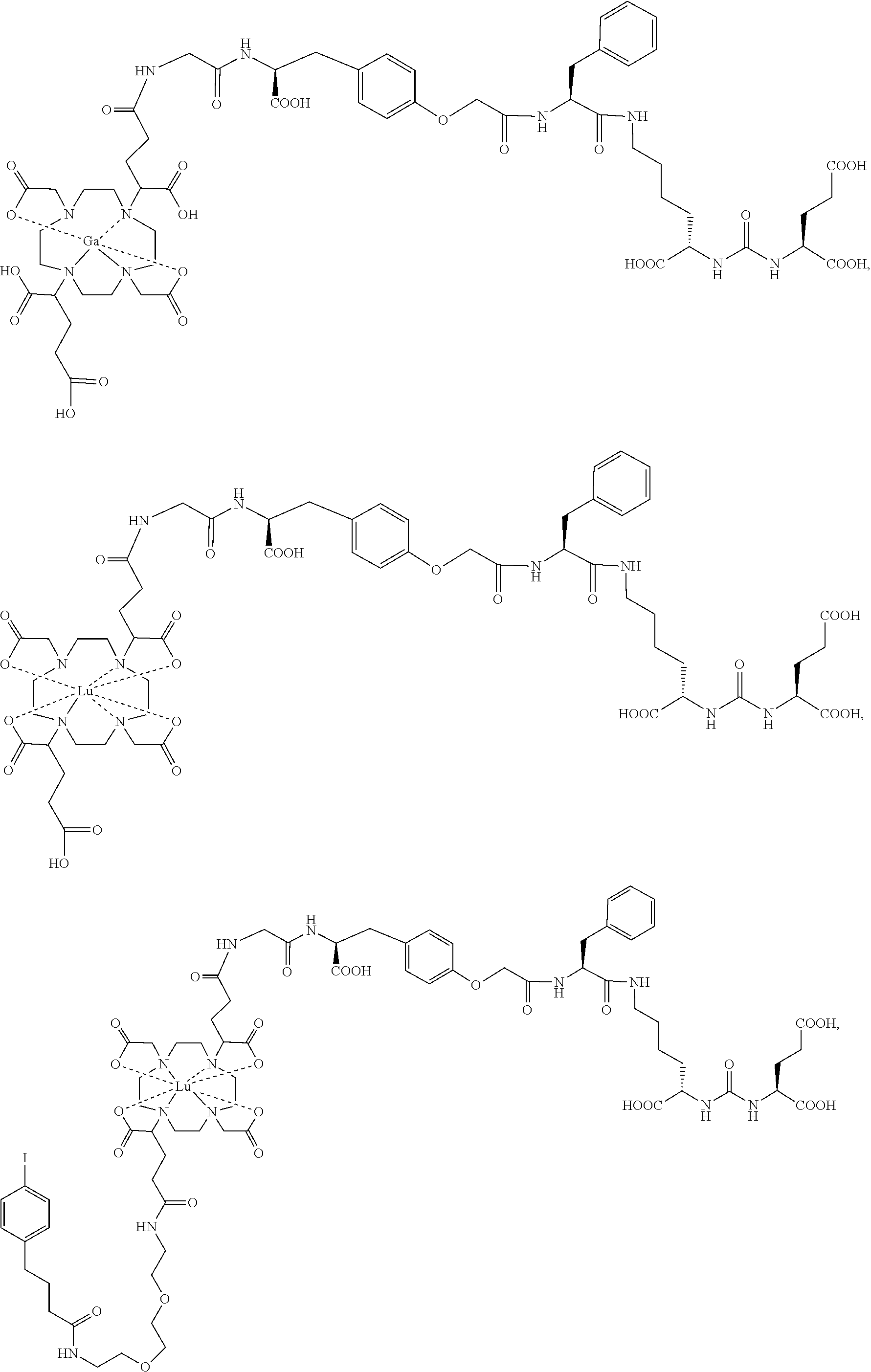

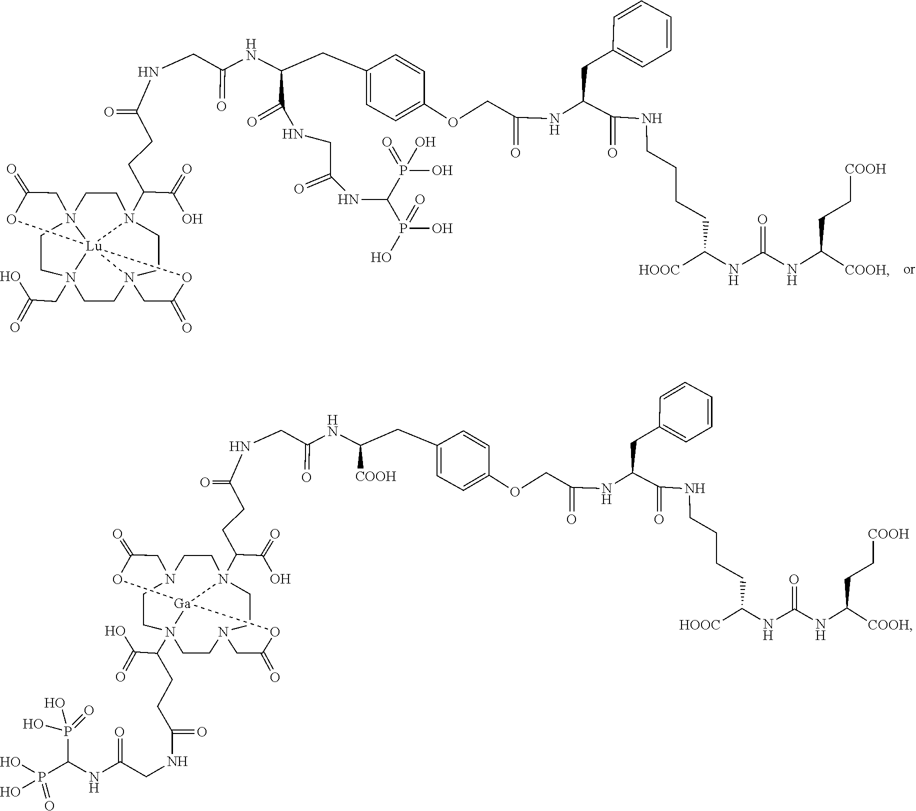

or a pharmaceutically acceptable salt thereof.

In one embodiment, the present disclosure relates to methods of making a compound of Formula I or a complex thereof.

In one embodiment, the present disclosure provides pharmaceutical compositions comprising a pharmaceutical acceptable carrier and a compound or complex disclosed herein. The present disclosure also provides pharmaceutical compositions comprising a pharmaceutical acceptable carrier and a pharmaceutically acceptable salt of a compound or complex disclosed herein.

In one embodiment, the present disclosure provides a kit formulation, comprising a sterile container containing a compound of Formula I or a pharmaceutically acceptable isotonic solution for i.v. injection thereof, and instructions for diagnostic imaging (for example, ⁶⁸Ga) and radiation therapy (for example, ¹¹⁷Lu) use.

The present disclosure also provides for methods of in vivo imaging, comprising administering an effective amount of a radiometal complex or a radioactive compound disclosed herein to a subject, and detecting the pattern of radioactivity of the complex or compound in the subject. In one embodiment, the disclosure relates to a method for imaging in a subject, comprising administering a radiolabeled compound disclosed herein to the subject; and obtaining an image of the subject or a portion of the subject. In another embodiment, the method for imaging comprises obtaining an image with a device that is capable of detecting positron emission.

The present disclosure also provides for methods of in vivo imaging, comprising administering an effective amount of a radiometal complex or a radioactive compound disclosed herein to a subject, and detecting the pattern of radioactivity of the complex or compound in said subject.

The present disclosure provide for methods of treating one or more tumors in a subject, comprising administering an effective amount of a radiometal complex or a radioactive compound disclosed herein to the subject. In some embodiments, the tumor is a PSMA-overexpressing tumor. In some embodiments, the tumor is prostate tumor, neuroendocrine tumor, or endocrine tumor. In some embodiments, the tumor is prostate tumor.

Typical subjects to which compounds of the disclosure may be administered will be mammals, particularly primates, especially humans. For veterinary applications, a wide variety of subjects will be suitable, e.g. livestock such as cattle, sheep, goats, cows, swine and the like; poultry such as chickens, ducks, geese, turkeys, and the like; and domesticated animals particularly pets such as dogs and cats. For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids and cell samples of the above subjects will be suitable for use such as mammalian, particularly primate such as human, blood, urine or tissue samples, or blood urine or tissue samples of the animals mentioned for veterinary applications.

Radiopharmaceuticals in accordance with this disclosure can be positron emitting gallium-68 complexes which, when used in conjunction with a ⁶⁸Ge/⁶⁸Ga parent/daughter radionuclide generator system, will allow PET imaging studies, avoiding the expense associated with operation of an in-house cyclotron for radionuclide production.

The complexes are formulated into aqueous solutions suitable for intravenous administration using standard techniques for preparation of parenteral diagnostics. An aqueous solution of the present complexes can be sterilized, for example, by passage through a commercially available 0.2 micron filter. The complexes are typically administered intravenously in an amount effective to provide tissue concentrations of the radionuclide complex sufficient to provide the requisite photon (gamma/positron) flux for imaging the tissue. The dosage level for any given complex of this disclosure to achieve acceptable tissue imaging depends on its particular biodistribution and the sensitivity of the tissue imaging equipment. Effective dosage levels can be ascertained by routine experimentation. They typically range from about 5 to about 30 millicuries. Where the complexes are gallium-68 complexes for PET imaging of myocardial tissue, adequate photon fluxes can be obtained by intravenous administration of from about 5 to about 30 millicuries of the complex.

The term “amino acid” used herein include both naturally occurring amino acids and unnatural amino acids. Naturally occurring amino acid refers to amino acids that are known to be used for forming the basic constituents of proteins, including alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and combinations thereof. Examples of unnatural amino acids include: an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; and a cyclic amino acid other than proline.

The term “alkanoyl” used herein refers to the following structure:

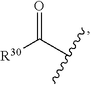

wherein R³⁰ is alkyl, cycloalkyl, aryl, (cycloalkyl)alkyl, or arylalkyl, any of which is optionally substituted. The acyl group can be, for example, C₁₋₆ alkylcarbonyl (such as, for example, acetyl), arylcarbonyl (such as, for example, benzoyl), levulinoyl, or pivaloyl. In another embodiment, the acyl group is benzoyl.

The term “alkyl” used herein includes both branched and straight-chain saturated aliphatic hydrocarbon groups, having the specified number of carbon atoms. Examples of alkyl include, but are not limited to, methyl, ethyl, n-propyl, i-propyl, n-butyl, s-butyl, t-butyl, n-pentyl, and s-pentyl. Preferred alkyl groups are C₁-C₁₀ alkyl groups. Typical C₁₋₁₀ alkyl groups include methyl, ethyl, n-propyl, n-butyl, n-pentyl, n-hexyl, n-heptyl, n-octyl, n-nonyl, and n-decyl, isopropyl, sec-butyl, tert-butyl, iso-butyl, iso-pentyl, neopentyl, 1-methylbutyl, 2-methylbutyl, 3-methylbutyl, 1,1-dimethylpropyl, 1,2-dimethylpropyl, 1-methylpentyl, 2-methylpentyl, 3-methylpentyl, 4-methylpentyl, 1-ethylbutyl, 2-ethylbutyl, 3-ethylbutyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-methylhexyl, 2-methylhexyl, 3-methylhexyl, 4-methylhexyl, 5-methylhexyl, 1,2-dimethylpentyl, 1,3-dimethylpentyl, 1,2-dimethylhexyl, 1,3-dimethylhexyl, 3,3-dimethylhexyl, 1,2-dimethylheptyl, 1,3-dimethylheptyl, and 3,3-dimethylheptyl, among others. In one embodiment, useful alkyl groups are selected from straight chain C₁₋₆ alkyl groups and branched chain C₃₋₆ alkyl groups. Typical C₁₋₆ alkyl groups include methyl, ethyl, propyl, isopropyl, butyl, sec-butyl, tert-butyl, iso-butyl, pentyl, 3-pentyl, hexyl, among others. In one embodiment, useful alkyl groups are selected from straight chain C₂₋₆ alkyl groups and branched chain C₃₋₆ alkyl groups. Typical C₂₋₆ alkyl groups include ethyl, propyl, isopropyl, butyl, sec-butyl, tert-butyl, iso-butyl, pentyl, 3-pentyl, hexyl among others. In one embodiment, useful alkyl groups are selected from straight chain C₁₋₄ alkyl groups and branched chain C₃₋₆ alkyl groups. Typical C₁₋₄ alkyl groups include methyl, ethyl, propyl, isopropyl, butyl, sec-butyl, tert-butyl, and iso-butyl.

The term “cycloalkyl” used herein includes saturated ring groups, having the specified number of carbon atoms, such as cyclopropyl, cyclobutyl, cyclopentyl, or cyclohexyl. Cycloalkyl groups typically will have 3 to about 12 ring members. In one embodiment, the cycloalkyl has one or two rings. In another embodiment, the cycloalkyl is a C₃-C₈ cycloalkyl. In another embodiment, the cycloalkyl is a C₃₋₇ cycloalkyl. In another embodiment, the cycloalkyl is a C₃₋₆ cycloalkyl. Exemplary cycloalkyl groups include cyclopropyl, cyclobutyl, cyclopentyl, cyclohexyl, cycloheptyl, cyclooctyl, norbornyl, decalin, and adamantyl.

The term “heterocycloalkyl” used herein refers to saturated heterocyclic alkyl groups.

The term “aryl” used herein includes C₆₋₁₄ aryl, especially C₆₋₁₀ aryl. Typical C₆₋₁₄ aryl groups include phenyl, naphthyl, phenanthryl, anthracyl, indenyl, azulenyl, biphenyl, biphenylenyl, and fluorenyl groups, more preferably phenyl, naphthyl, and biphenyl groups.

The term “heteroaryl” or “heteroaromatic” used herein refers to groups having 5 to 14 ring atoms, with 6, 10 or 14 π electrons shared in a cyclic array, and containing carbon atoms and 1, 2, or 3 oxygen, nitrogen or sulfur heteroatoms, or 4 nitrogen atoms. In one embodiment, the heteroaryl group is a 5- to 10-membered heteroaryl group. Examples of heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. Typical heteroaryl groups include thienyl (e.g., thien-2-yl and thien-3-yl), furyl (e.g., 2-furyl and 3-furyl), pyrrolyl (e.g., pyrrol-1-yl, 1H-pyrrol-2-yl and 1H-pyrrol-3-yl), imidazolyl (e.g., imidazol-1-yl, 1H-imidazol-2-yl and 1H-imidazol-4-yl), tetrazolyl (e.g., tetrazol-1-yl and tetrazol-5-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, pyrimidin-5-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). A 5-membered heteroaryl can contain up to 4 heteroatoms. A 6-membered heteroaryl can contain up to 3 heteroatoms. Each heteroatom is independently selected from nitrogen, oxygen and sulfur.

Suitable carboxylic acid protecting group are well known and include, for example, any suitable carboxylic acid protecting group disclosed in Wuts, P. G. M. & Greene, T. W., Greene's Protective Groups in Organic Synthesis, 4rd Ed., pp. 16-430 (J. Wiley & Sons, 2007), herein incorporated by reference in its entirety. Those skilled in the art will be familiar with the selection, attachment, and cleavage of protecting groups and will appreciate that many different protective groups are known in the art, the suitability of one protective group or another being dependent on the particular synthetic scheme planned. Suitable carboxylic acid protecting group include, for example, the methyl esters, t-butyl esters, benzyl esters, and allyl esters.

### Materials and Methods for Synthesis

### General

All reagents and solvents were purchased commercially (Aldrich, Acros, or Alfa

Inc.) and were used without further purification, unless otherwise indicated. Solvents were dried through a molecular sieve system (Pure Solve Solvent Purification System; Innovative Technology, Inc.). ¹H and ¹³C NMR spectra were recorded on a Bruker Avance spectrometer at 400 MHz and 100 MHz, respectively, and referenced to NMR solvents as indicated. Chemical shifts are reported in ppm (δ), with a coupling constant, J, in Hz. The multiplicity is defined by singlet (s), doublet (d), triplet (t), broad (br), and multiplet (m). High-resolution mass spectrometry (HRMS) data was obtained with an Agilent (Santa Clara, Calif.) G3250AA LC/MSD TOF system. Thin-layer chromatography (TLC) analyses were performed using Merck (Darmstadt, Germany) silica gel 60 F₂₅₄ plates. Generally, crude compounds were purified by flash column chromatography (FC) packed with silica gel (Aldrich). High performance liquid chromatography (HPLC) was performed on an Agilent 1100 series system. A gamma counter (Cobra II auto-gamma counter, Perkin-Elmer) measured ⁶⁸Ga radioactivity. Reactions of non-radioactive chemical compounds were monitored by thin-layer chromatography (TLC) analysis with pre-coated plates of silica gel 60 F₂₅₄. An aqueous solution of [⁶⁸Ga]GaCl₃ was obtained from a ⁶⁸Ge/⁶⁸Ga generator (Radiomedix Inc.). Solid-phase extraction cartridges (SEP Pak® Light QMA, Oasis® HLB 3cc) were obtained from Waters (Milford, Mass., USA).

Compounds 4, 7, 17, 18, 26, 27, 29, 38, 42, and 51, all containing the urea-Glu group (Glu-NH—CO—NH—), were prepared as described in the following sections. It is noted that PSMA-11 and MIP-1095 are known PSMA imaging agents, and they are presented as a positive control for binding to PSMA.

Preparation of the intermediate compound 2 was based on the following chemical reactions (Scheme 8) and described in U.S. Patent Application Publication No. 2017/0189568, which is incorporated herein by reference in its entirety.

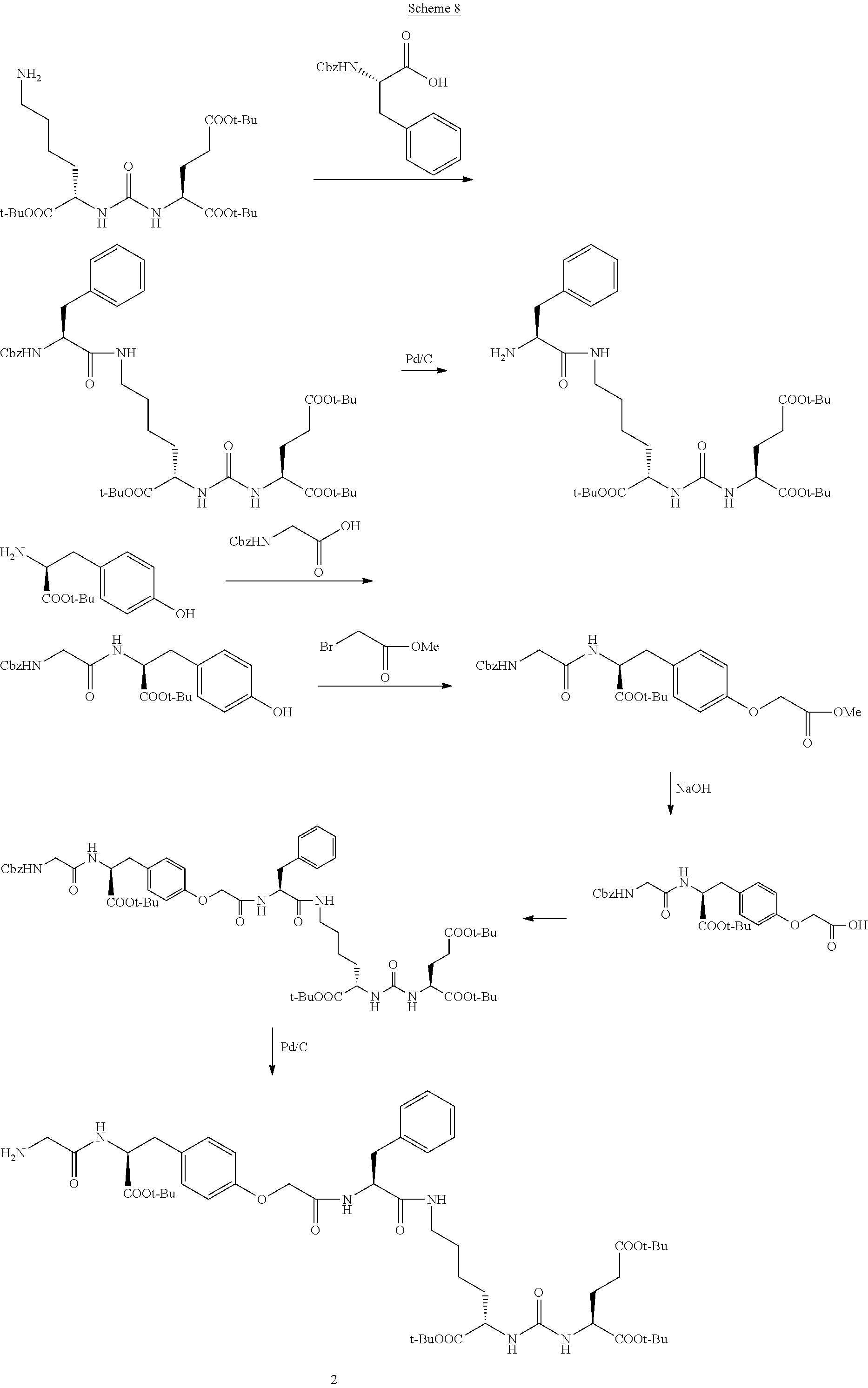

Preparation of compound 4 was based on the following chemical reactions (Scheme 9). Compounds 1 and 2 were synthesized according to known methods [5].

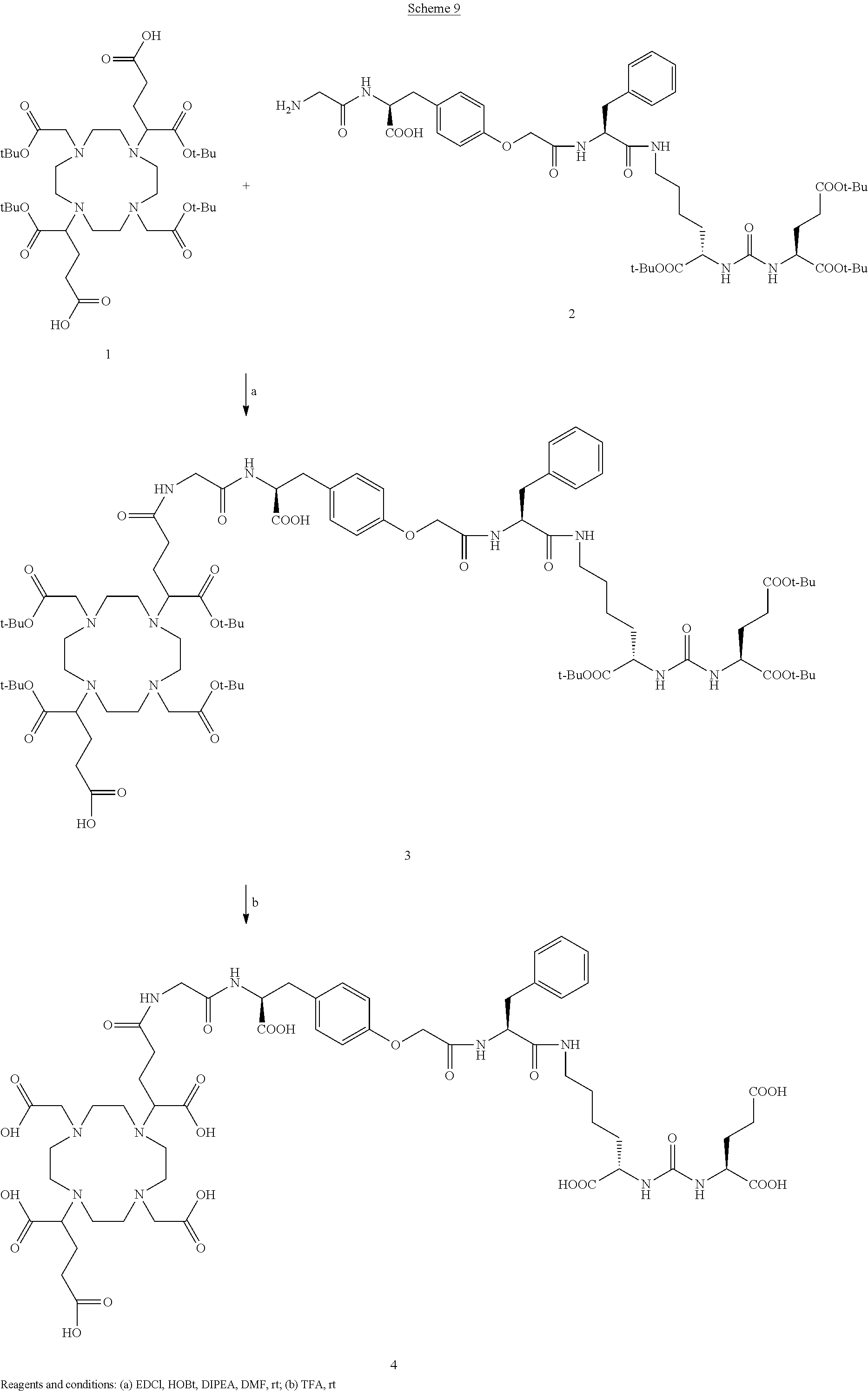

Preparation of compound 7 was based on the following chemical reactions (Scheme 10).

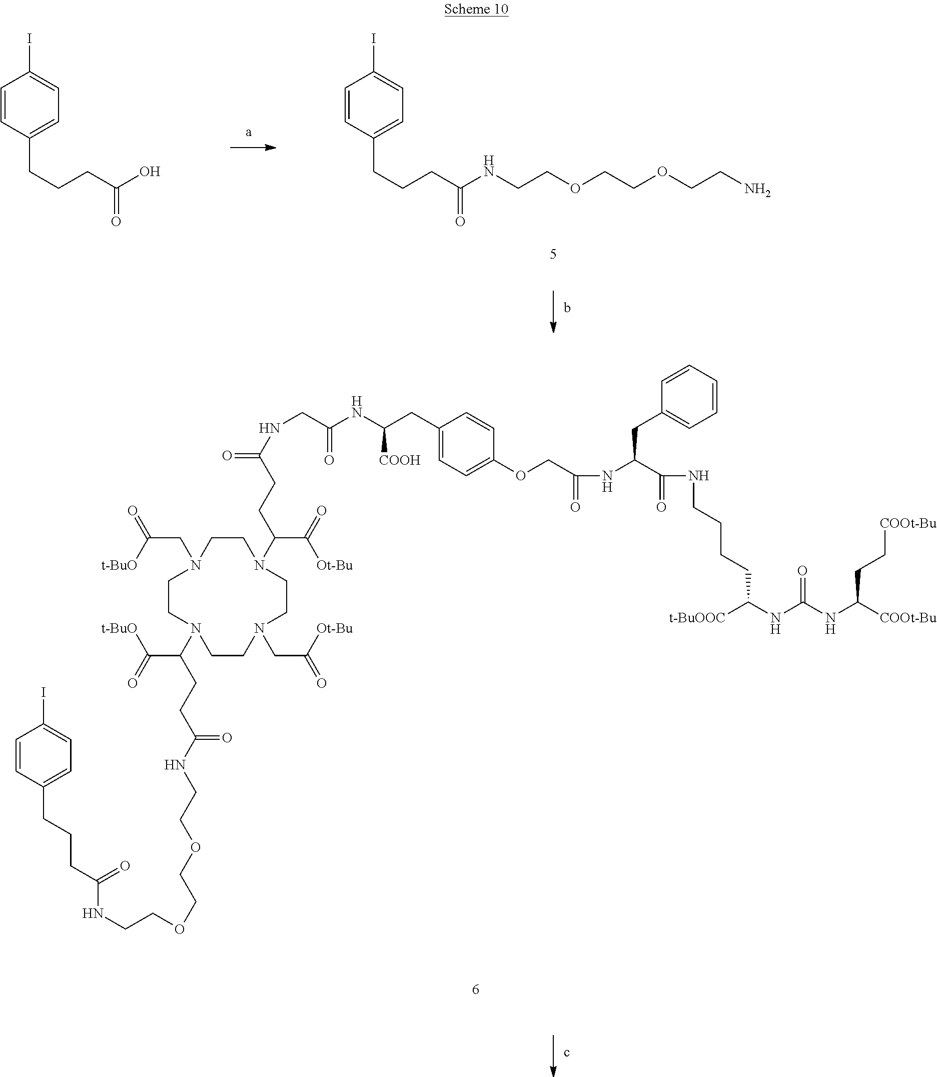

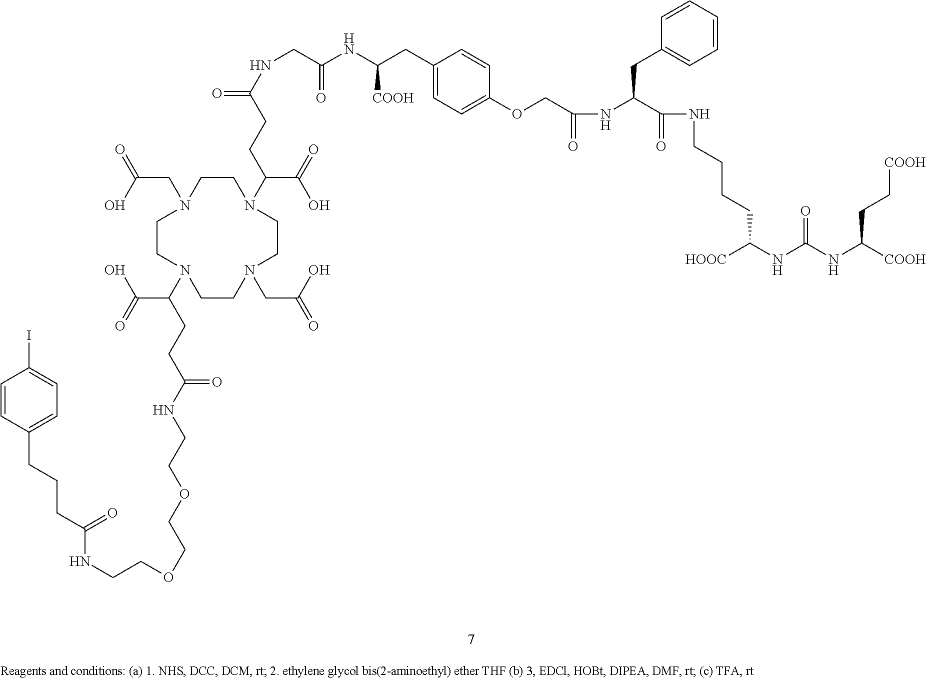 [SMILES: O=C(O)CC[C@H](NC(=O)N[C@@H](CCCCNC(=O)[C@H](Cc1ccccc1)NC(=O)COc1ccc(C[C@H](NC(=O)CNC(=O)CCC(C(=O)O)N2CCN(CC(=O)O)CCN(C(CCC(=O)NCCOCCOCCNC(=O)CCCc3ccc(I)cc3)C(=O)O)CCN(CC(=O)O)CC2)C(=O)O)cc1)C(=O)O)C(=O)O]

### EXAMPLE 1

### 4-(7-(5-((2-(((S)-2-(4-(((4S,11S,15S)-4-benzyl-11,15-bis(tert-butoxycarbonyl)-20,20-dimethyl-2,5,13,18-tetraoxo-19-oxa-3,6,12,14-tetraazahenicosyl)oxy)phenyl)-1-carboxyethyl)amino)-2-oxoethyl)amino)-1-(tert-butoxy)-1,5-dioxopentan-2-yl)-4,10-bis(2-(tert-butoxy)-2-oxoethyl)-1,4,7,10-tetraazacyclododecan-1-yl)-5-(tert-butoxy)-5-oxopentanoic acid (3)

To a solution of 2 (124 mg, 0.129 mmol) in 5 mL DMF, N,N-diisopropylethylamine (DIPEA, 49 mg, 0.38 mmol), 1-hydroxybenzotriazole hydrate (HOBt, 32.7 mg, 0.19 mmol), N-(3-dimethylaminopropyl)-N-ethylcarbodiimide hydrochloride (EDC, 37 mg, 0.19 mmol) and 1 (100 mg, 0.129 mmol) were added at 0° C. The mixture was stirred at rt overnight before 30 mL EtOAc were added to the reaction mixture. It was then washed with H2O (10 mL×2) and brine (10 mL), dried over MgSO4, and filtered. The filtrate was concentrated, and the residue was purified by FC (DCM/MeOH/NH4OH=90/9/1) to give 40 mg 3 as colorless oil. (yield: 17.6%).

### EXAMPLE 2

### (4S,11S,15S)-4-benzyl-1-(4-((2S)-2-(2-(4-(4,10-bis(carboxymethyl)-7-(1,3-dicarboxypropyl)-1,4,7,10-tetraazacyclododecan-1-yl)-4-carboxybutanamido)acetamido)-2-carboxyethyl)phenoxy)-2,5,13-trioxo-3,6,12,14-tetraazaheptadecane-11,15,17-tricarboxylic acid (4)

A solution of 3 (20 mg, 0.011 mmol) in 1 mL TFA was stirred at rt for 5 h. The reaction mixture was evaporated in vacuo, and the residue was recrystallized from Ether/EtOH. The resulting white solid was dissolved in 1 mL MeOH and purified by semi prep-HPLC to give 5 as a yellow oil (yield: 10 mg, 71.3%): ¹HNMR(400 MHz, MeOD) δ: 7.16-7.29(m, 7H), 6.85-6.89(m, 2H), 4.65-4.67(m, 2H), 4.45-4.55(m, 2H), 4.31-4.34(m, 2H), 4.23-4.24(m, 4H), 2.95-3.92(m, 25 H), 2.62-2.70(m, 4H), 2.40-2.45(m, 2H), 1.62-2.17(m, 8H), 1.36-1.47(m, 4H); HRMS calcd for C₅₆H₇₉N₁₀O₂₄ (M+H)⁺, 1275.5269; found 1275.5338.

### EXAMPLE 3

### N-(2-(2-(2-aminoethoxy)ethoxy)ethyl)-4-(4-iodophenyl)butanamide (5)

To a solution of 4-(p-iodophenyl)butyric acid (145 mg, 0.5 mmol) in 5 mL DCM was added NHS (69 mg, 0.6 mmol) and DCC (125 mg, 0.6 mmol). The reaction was stirred at rt for 2 h. 20 mL THF was then added into the mixture, followed by ethylene glycol bis(2-aminoethyl) ether (210 mg, 1.5 mmol). The reaction mixture was then stirred at rt overnight and the solvent was removed, and the residue was purified by FC (DCM/MeOH/NH₄OH=90/9/1) to give 120 mg 5 as colorless oil. (yield: 57.1%). ¹HNMR(400 MHz, MeOD) δ: 7.61(d, 2H, J=8.0 Hz), 6.96(d, 2H, J=8.0 Hz), 6.24(br S, 1H), 3.52-3.60(m, 8H), 3.45-3.49(m, 2H), 2.87-2.89(m, 2H), 2.60-2.64(m, 2H), 2.17-2.21(m, 2H), 1.94-1.98(m, 2H).

### EXAMPLE 4

### (2S)-3-(4-(((4S,11S,15S)-4-benzyl-11,15-bis(tert-butoxycarbonyl)-20,20-dimethyl-2,5,13,18-tetraoxo-19-oxa-3,6,12,14-tetraazahenicosyl)oxy)phenyl)-2-(2-(4-(4,10-bis(2-(tert-butoxy)-2-oxoethyl)-7-(22-(4-iodophenyl)-2,2-dimethyl-4,8,19-trioxo-3,12,15-trioxa-9,18-diazadocosan-5-yl)-1,4,7,10-tetraazacyclododecan-1-yl)-5-(tert-butoxy)-5-oxopentanamido)acetamido)propanoic acid (6)

To a solution of 3 (10 mg, 0.01 mmol) in 5 mL DMF, N,N-diisopropylethylamine (DIPEA, 3.9 mg, 0.07 mmol), 1-hydroxybenzotriazole hydrate (HOBt, 2 mg, 0.015 mmol), N-(3-dimethylaminopropyl)-N-ethylcarbodiimide hydrochloride (EDC, 2.9 mg, 0.015 mmol) and 5 (4.2 mg, 0.01 mmol) were added at 0° C. The mixture was stirred at rt overnight before 30 mL EtOAc were added to the reaction mixture. It was then washed with H₂O (10 mL×2) and brine (10 mL), dried over MgSO₄, and filtered. The filtrate was concentrated, and the residue was purified by FC (DCM/MeOH/NH₄OH=90/9/1) to give 20 mg 6 as colorless oil. (yield: 92%).

### EXAMPLE 5

### (4S,11S,15S)-4-benzyl-1-(4-((2S)-2-carboxy-2-(2-(4-carboxy-4-(7-(1-carboxy-18-(4-iodophenyl)-4,15-dioxo-8,11-dioxa-5,14-diazaoctadecyl)-4,10-bis(carboxymethyl)-1,4,7,10-tetraazacyclododecan-1-yl)butanamido)acetamido)ethyl)phenoxy)-2,5,13-trioxo-3,6,12,14-tetraazaheptadecane-11,15,17-tricarboxylic acid (7)

A solution of 6 (20 mg, 0.0092 mmol) in 1 mL TFA was stirred at rt for 5 h. The reaction mixture was evaporated in vacuo, and the residue was recrystallized from Ether/EtOH. The resulting white solid was dissolved in 1 mL MeOH and purified by semi prep-HPLC to give 7 as a yellow oil (yield: 12 mg, 77.8%): 1HNMR(400 MHz, MeOD) δ: 7.62(d, 2H, J=7.6 Hz), 7.16-7.29(m, 7H), 7.01(d, 2H, J=7.6 Hz), 6.88(m, 2H), 4.66-4.67(m, 2H), 4.45-4.55(m, 2H), 4.32(m, 2H), 4.24(m, 2H), 3.00-3.98(m, 35H), 2.59-2.67(m, 8H), 2.43(m, 2H), 2.20-2.36(m, 2H), 1.64-2.16(m, 10H), 1.35-1.54(m, 4H); HRMS calcd for C72H102IN12O26 (M+H)+, 1677.6073; found 1677.6157.

Preparation of compounds 17 and 18 was based on the following chemical reactions (Scheme 11)

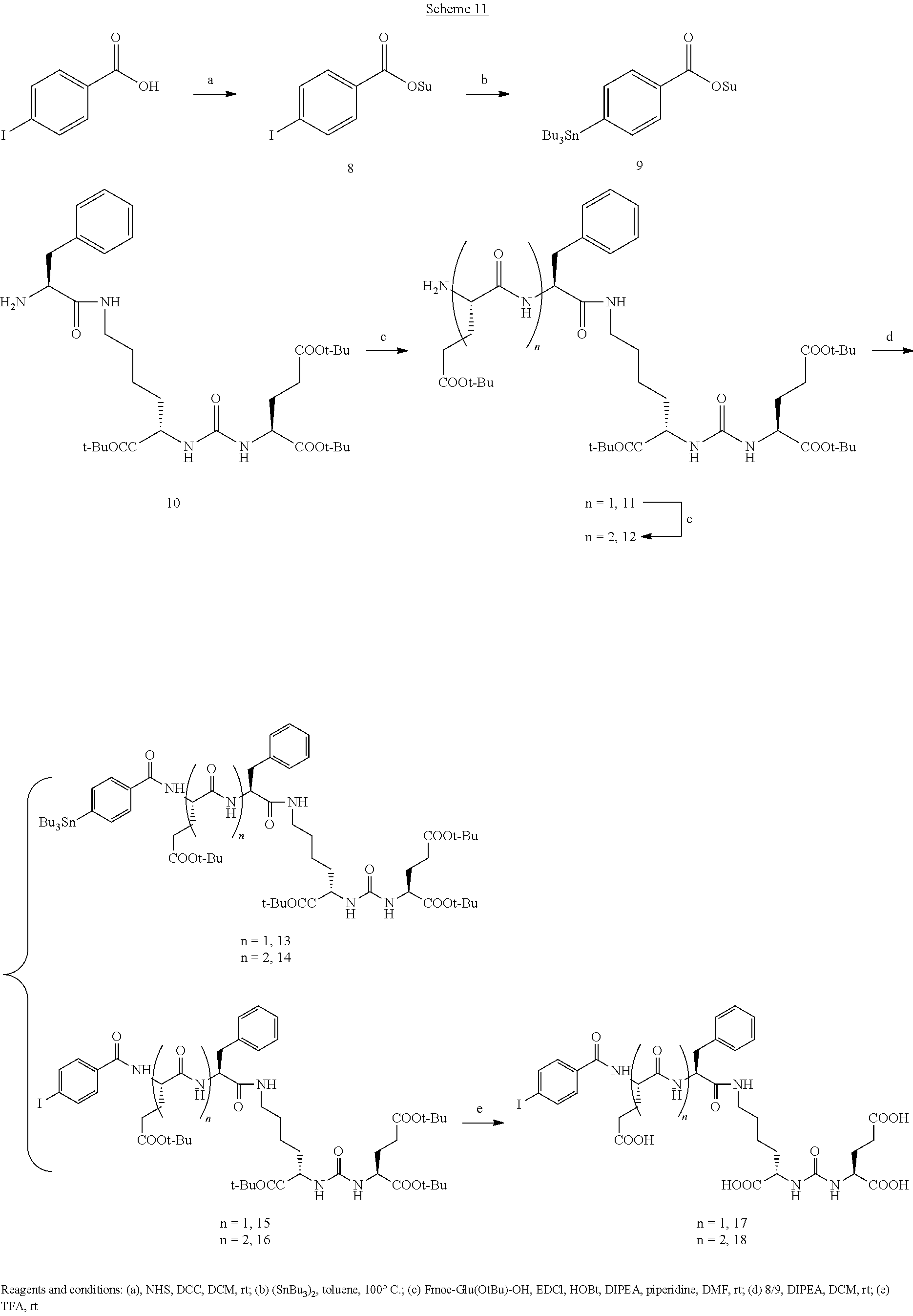

Di-tert-butyl (((S)-6-((S)-2-((S)-2-amino-5-(tert-butoxy)-5-oxopentanamido)-3-phenylpropanamido)-1-(tert-butoxy)-1-oxohexan-2-yl)carbamoyl)-L-glutamate (11).

To a solution of 10 (440 mg, 0.69 mmol) in 10 mL DMF, N,N-diisopropylethylamine (DIPEA, 267 mg, 2.07 mmol), 1-hydroxybenzotriazole hydrate (HOBt, 175 mg, 1 mmol), N-(3-dimethylaminopropyl)-N-ethylcarbodiimide hydrochloride (EDC, 191 mg, 1 mmol) and Fmoc-Glu(OtBu)-OH (300 mg, 0.69 mmol) were added at 0° C. After stirred at rt overnight, 1 mL piperidine was added into the mixture and maintained at rt for 2 h. 50 mL EtOAc were added to the reaction mixture. It was then washed with H₂O (20 mL×2) and brine (20 mL), dried over MgSO₄, and filtered. The filtrate was concentrated, and the residue was purified by FC (DCM/MeOH/NH₄OH=90/9/1) to give 366 mg 11 as colorless oil. (yield: 64.8%). HRMS calcd for C₄₂H₇₀N₅O₁₁ (M+H)⁺, 820.5072; found 820.5103.

Di-tert-butyl (((S)-6-((S)-2-((S)-2-((S)-2-amino-5-(tert-butoxy)-5-oxopentanamido)-5-(tert-butoxy)-5-oxopentanamido)-3-phenylpropanamido)-1-(tert-butoxy)-1-oxohexan-2-yl)carbamoyl)-L-glutamate (12).

Compound 12 was prepared from 11 (266 mg, 0.32 mmol), N,N-diisopropylethylamine (DIPEA, 123 mg, 0.96 mmol), 1-hydroxybenzotriazole hydrate (HOBt, 81 mg, 0.48 mmol), N-(3-dimethylaminopropyl)-N-ethylcarbodiimide hydrochloride (EDC, 91 mg, 0.48 mmol) and Fmoc-Glu(OtBu)-OH (143 mg, 0.32 mmol) following the same procedure described for compound 11. Compound 12: 159 mg (yield: 49.4%). HRMS calcd for C₅₁H₈₅N₆O₁₄ (M+H)⁺, 1005.6124; found 1005.6087.

Di-tert-butyl (((S)-1-(tert-butoxy)-6-((S)-2-((S)-5-(tert-butoxy)-5-oxo-2-(4-(tributylstannyl)benzamido)pentanamido)-3-phenylpropanamido)-1-oxohexan-2-yl)carbamoyl)-L-glutamate (13).

To a solution of 11 (43 mg, 0.05 mmol) in 10 mL DMF, DIPEA (10 mg, 0.08 mmol) and 9 (37 mg, 0.06 mmol) were added at 0° C. The mixture was stirred at rt for 5 h and the solvent was removed in vacuo. The residue was purified by FC (DCM/MeOH/NH₄OH=95/5/0.5) to give 17.7 mg 13 as colorless oil. (yield: 28.1%). ¹HNMR(400 MHz, CDCl₃) δ: 8.03(d, 1H, J=4.4 Hz), 7.76(d, 2H, J=6.4 Hz), 7.48-7.59(m, 2H), 7.15(s, 4H), 7.09(s, 1H), 6.91-6.97(m, 2H), 5.99(d, 1H, J=7.6 Hz), 5.79(d, 1H, J=8.4 Hz), 5.31(s, 1H), 4.53-4.60(m, 2H), 4.29-4.34(m, 2H), 3.06-3.35(m, 4H), 2.30-2.37(m, 4H), 2.04-2.09(m, 3H), 1.79-1.87(m, 1H), 1.53-1.59(m, 6H), 1.42-1.45(m, 40H), 1.29-1.37(m, 6H), 1.08-1.12(m, 6H), 0.88-0.91(m, 9H); HRMS calcd for C₆₁H₉₉N₅NaO₁₂Sn (M+Na)⁺, 1236.6210; found 1236.6248.

Di-tert-butyl (((S)-1-(tert-butoxy)-6-((S)-2-((S)-5-(tert-butoxy)-2-((S)-5-(tert-butoxy)-5-oxo-2-(4-(tributylstannyl)benzamido)pentanamido)-5-oxopentanamido)-3-phenylpropanamido)-1-oxohexan-2-yl)carbamoyl)-L-glutamate (14).

To a solution of 12 (40 mg, 0.04 mmol) in 10 mL DCM, DIPEA (77 mg, 0.06 mmol) and 9 (24 mg, 0.048 mmol) were added at 0° C. The mixture was stirred at rt overnight and the solvent was removed in vacuo. The residue was purified by FC (DCM/MeOH/NH₄OH=95/5/0.5) to give 25.6 mg 14 as colorless oil. (yield: 45.8%). ¹HNMR(400 MHz, MeOD) δ: 8.82(d, 1H, J=3.6 Hz), 8.70(d, 1H, J=6.4 Hz), 7.92(d, 2H, J=6.4 Hz), 7.51-7.62(m, 3H), 7.11-7.17(m, 5H), 6.86(s, 1H), 6.36(d, 1H, J=8.0 Hz), 5.53(d, 1H, J=7.2 Hz), 4.80-4.84(m, 1H), 4.30-4.45(m, 4H), 3.62-3.65(m, 1H), 3.37-3.39(m, 1H), 3.20-3.25(m, 1H), 2.97-3.03(m, 1H), 2.65-2.69(m, 1H), 2.50-2.57(m, 1H), 2.24-2.30(m, 5H), 2.03-2.08(m, 2H), 1.62-1.85(m, 5H), 1.38-1.56(m, 55H), 1.07-1.11(m, 6H), 0.88-0.91(m, 9H); HRMS calcd for C₇₀H₁₁₄N₆NaO₁₅Sn (M+Na)⁺, 1421.7262; found 1421.7242.

Di-tert-butyl (((S)-1-(tert-butoxy)-6-((S)-2-((S)-5-(tert-butoxy)-2-(4-iodobenzamido)-5-oxopentanamido)-3-phenylpropanamido)-1-oxohexan-2-yl)carbamoyl)-L-glutamate (15).

Compound 15 was prepared from 12 (37 mg, 0.045 mmol), DIPEA (9 mg, 0.07 mmol) and 8 (19 mg, 0.054 mmol), following the same procedure described for compound 13. Compound 15: 24 mg (yield: 50.7%). ¹HNMR(400 MHz, CDCl₃) δ: 8.12(d, 1H, J=5.6 Hz), 7.77(d, 2H, J=7.6 Hz), 7.57(d, 2H, J=7.6 Hz), 7.09-7.16(m, 6H), 6.94(s, 1H), 5.99(d, 1H, J=4.8Hz), 5.83(d, 1H, J=8.0 Hz), 4.53-4.61(m, 2H), 4.15-4.36(m, 2H), 3.39(d, 1H, J=7.6 Hz), 3.01-3.22(m, 2H), 2.98-3.04(m, 1Hz), 2.28-2.41(m, 4H), 2.00-2.07(m, 3H), 1.50-1.85(m, 3H), 1.42-1.45(m, 40H); HRMS calcd for C₄₉H₇₃IN₅O₁₂ (M+H)⁺, 1050.4300; found 1050.4326.

Di-tert-butyl (((S)-1-(tert-butoxy)-6-((S)-2-((S)-5-(tert-butoxy)-2-((S)-5-(tert-butoxy)-2-(4-iodobenzamido)-5-oxopentanamido)-5-oxopentanamido)-3-phenylpropanamido)-1-oxohexan-2-yl)carbamoyl)-L-glutamate (16).

Compound 16 was prepared from 12 (40 mg, 0.04 mmol), DIPEA (26 mg, 0.048 mmol) and 8 (17 mg, 0.048 mmol), following the same procedure described for compound 13. Compound 16: 40 mg (yield: 80.1%). ¹HNMR(400 MHz, MeOD) δ: 8.87(d, 1H, J=3.6 Hz), 8.81(d, 1H, J=6.4 Hz), 7.82(d, 2H, J=8.4 Hz), 7.72(d, 2H, J=8.4 Hz), 7.50(d, 1H, J=8.8 Hz), 7.11-7.17(m, 5H), 6.92(s, 1H), 6.31(d, 1H, J=8.4 Hz), 5.52(d, 1H, J=7.6 Hz), 4.72-4.83(m, 1H), 4.31-4.42(m, 4H), 3.59-3.63(m, 1H), 3.32-3.40(m, 1H), 3.20-3.25(m, 1H), 2.94-3.01(m, 1H), 2.56-2.65(m, 1H), 2.45-2.50(m, 1H), 2.10-2.32(m, 5H), 2.01-2.08(m, 2H), 1.62-1.88(m, 5H), 1.41-1.56(m, 49H); HRMS calcd for C₅₈H₈₈IN₆O₁₅ (M+H)⁺, 1235.5352; found 1235.5422.

(((S)-1-Carboxy-5-((S)-2-((S)-4-carboxy-2-(4-iodobenzamido)butanamido)-3-phenylpropanamido)pentyl)carbamoyl)-L-glutamic acid (17).

Compound 17 was prepared from 15 (17 mg, 0.016 mmol) in 1 mL TFA, following the same procedure described for compound 4. Compound 17: 8.6 mg (yield: 64.2%). ¹HNMR(400 MHz, MeOD) δ: 7.86(d, 2H, J=7.6 Hz), 7.61(d, 2H, J=8.0 Hz), 7.18(s, 4H), 7.15(s, 1H), 4.54-4.57(m, 1H), 4.46-4.49(m, 1H), 4.21-4.30(m, 2H), 3.58-3.60(m, 2H), 3.47-3.52(m, 1H), 3.11-3.16(m, 3H), 2.95-3.00(m, 1H), 2.34-2.41(m, 4H), 1.99-2.17(m, 4H), 1.75-1.77(m, 1H), 1.60-1.64(m, 1H), 1.43-1.45(m, 2H), 1.12-1.27(m, 2H); HRMS calcd for C₃₃H₄₁IN₅O₁₂ (M+H)⁺, 826.1796; found 826.1755.

(((S)-1-Carboxy-5-((S)-2-((S)-4-carboxy-2-((S)-4-carboxy-2-(4-iodobenzamido)butanamido)butanamido)-3-phenylpropanamido)pentyl)carbamoyl)-L-glutamic acid (18).

Compound 18 was prepared from 16 (38 mg, 0.031 mmol) in 1 mL TFA, following the same procedure described for compound 4. Compound 18: 10.1 mg (yield: 34.1%). ¹HNMR(400 MHz, MeOD) δ: 8.51(d, 1H, J=6.4 Hz), 7.98(d, 1H, J=8.4 Hz), 7.86(d, 2H, J=8.4 Hz), 7.71(s, 1H), 7.66(d, 2H, J=8.4 Hz), 7.16-7.22(m, 5H), 4.54-4.58(m, 1H), 4.45-4.48(m, 1H), 4.26-4.31(m, 3H), 3.15-3.21(m, 3H), 3.15-3.21(m, 1H), 2.49-2.52(m, 2H), 2.31-2.41(m, 2H), 2.25-2.28(m, 1H), 2.08-2.19(m, 4H), 1.77-1.97(m, 4H), 1.62-1.68(m, 1H), 1.44-1.49(m, 2H), 1.34-1.39(m, 2H); HRMS calcd for C₃₈H₄₈IN₆O₁₅ (M+H)⁺, 955.2222; found 955.2273.

Preparation of compounds 26 and 27 was based on the following chemical reactions (Scheme12)

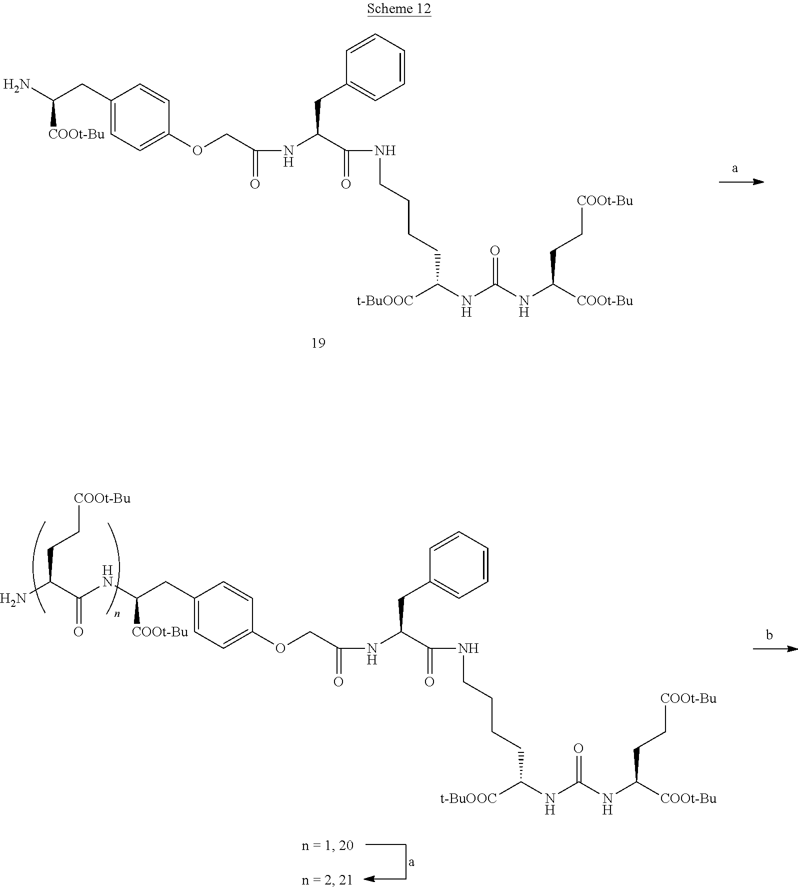

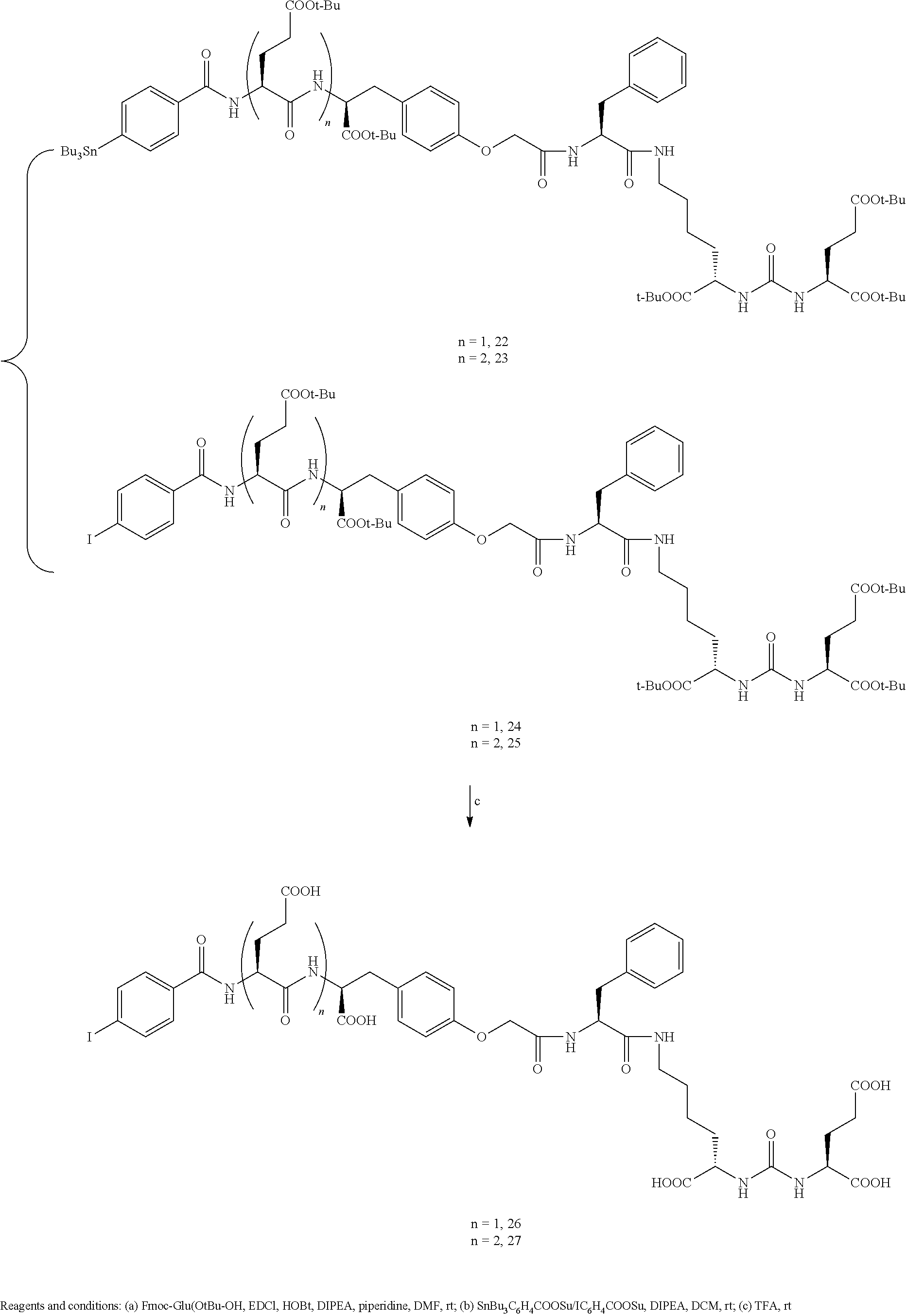

Di-tert-butyl (((S)-6-(S)-2-(2-(4-((S)-2-((S)-2-amino-5-(tert-butoxy)-5-oxopentanamido)-3 -(tert-butoxy)-3 -oxopropyl)phenoxy)acetamido)-3-phenylpropanamido)-1-(tert-butoxy)-1-oxohexan-2-yl)carbamoyl)-L-glutamate (20).

Compound 20 was prepared from 19 (455 mg, 0.5 mmol), DIPEA, (193 mg, 1.5 mmol), HOBt(127 mg, 0.75 mmol), EDC(142 mg, 0.75 mmol) and Fmoc-Glu(OtBu)-OH (221 mg, 0.5 mmol) following the same procedure described for compound 11. Compound 20: 361 mg (yield: 65.8%). HRMS calcd for C₅₇H₈₉N₆O₁₅ (M+H)⁺, 1097.6386; found 1097.6399.

Di-tert-butyl (((S)-6-(S)-2-(2-(4((S)-2-((S)-2-((S)-2-amino-5-(tert-butoxy)-5-oxopentanamido)-5-(tert-butoxy)-5-oxopentanamido)-3-(tert-butoxy)-3-oxopropyl)phenoxy)acetamido)-3-phenylpropanamido)-1-(tert-butoxy)-1-oxohexan-2-yl)carbamoyl)-L-glutamate (21).

Compound 21 was prepared from 20 (220 mg, 0.2 mmol), DIPEA, (78 mg, 0.6 mmol), HOBt(51 mg, 0.3 mmol), EDC(57 mg, 0.3 mmol) and Fmoc-Glu(OtBu)-OH (88 mg, 0.2 mmol) following the same procedure described for compound 11. Compound 21: 156 mg (yield: 60.8%). HRMS calcd for C₆₆H₁₀₄N₇O₁₈ (M+H)⁺, 1282.7438; found 1282.7511.

Di-tert-butyl (((S)-1-(tert-butoxy)-6-((S)-2-(2-(4-((S)-3-(tert-butoxy)-2-((S)-5-(tert-butoxy)-5-oxo-2-(4-(tributylstannyl)benzamido)pentanamido)-3-oxopropyl)phenoxy)acetamido)-3-phenylpropanamido)-1-oxohexan-2-yl)carbamoyl)-L-glutamate (22).

Compound 22 was prepared from 20 (76 mg, 0.07 mmol), DIPEA (27 mg, 0.21 mmol) and 9 (69.4 mg, 0.14 mmol), following the same procedure described for compound 13. Compound 22: 33.6 mg (yield: 48.0%). ¹HNMR(400 MHz, CD₂Cl₂) δ: 7.70(d, 2H, J=6.8 Hz), 7.51(d, 2H, J=7.2 Hz), 7.38(d, 2H, J=6.4 Hz), 7.62-7.30(m, 2H), 7.19-7.23(m, 1H), 6.88(d, 2H, J=7.6 Hz), 6.54(d, 2H, J=7.6 Hz), 5.55(d, 1H, J=8.4 Hz), 4.76(s, 1H), 4.48(s, 1H), 4.25(s, 1H), 3.16-3.40(m, 5H), 2.97-3.08(m, 2H), 2.25-2.47(m, 5H), 2.10-2.17 (m, 3H), 1.87-1.95(m, 2H), 1.51-1.57(m, 13H), 1.43(d, 25H, J=11.2 Hz), 1.27-1.36(m, 18H), 1.12-1.27(m, 7H), 1.08-1.12(m, 6H), 0.87-0.92(m, 9H); HRMS calcd for C₇₆H₁₁₈N₆NaO₁₆Sn (M+Na)⁺, 1513.7524; found 1513.7674.

Di-tert-butyl (((S)-1-(tert-butoxy)-6-((S)-2-(2-(4-((S)-3-(tert-butoxy)-2-((S)-5-(tert-butoxy)-2-((S)-5-(tert-butoxy)-5-oxo-2-(4-(tributylstannyl)benzamido)pentanamido)-5-oxopentanamido)-3-oxopropyl)phenoxy)acetamido)-3-phenylpropanamido)-1-oxohexan-2-yl)carbamoyl)-L-glutamate (23).

Compound 23 was prepared from 21 (50 mg, 0.04 mmol), DIPEA (6 mg, 0.048 mmol) and 9 (13.8 mg, 0.04 mmol), following the same procedure described for compound 13. Compound 23: 35 mg (yield: 57.8%). ¹HNMR(400 MHz, CDCl₃) δ: 7.81(d, 2H, J=6.4 Hz), 7.54-7.56(m, 3H), 7.32-7.34(m, 1H), 7.19-7.28(m, 5H), 7.09-7.11(m, 3H), 6.76-6.78(m, 3H), 6.08(s, 1H), 5.69(d, 1H, J=7.2 Hz), 4.80-4.82(m, 1H), 4.63-4.69(m, 2H), 4.36-4.51(m, 5H), 3.37-3.39(m, 1H), 2.96-3.12(m, 5H), 2.52-2.56(m, 1H), 2.32-2.43(m, 5H), 2.01-2.20(m, 6H), 1.75-1.84(m, 2H), 1.28-1.55(m, 64H), 1.07-1.11(m, 6H), 0.88-0.92(m, 9H); HRMS calcd for C₈₅H₁₃₃NaN₇O₁₉Sn (M+H)⁺, 1698.8576; found 1698.8774.

Di-tert-butyl (O)-1-(tert-butoxy)-6-(S)-2-(2-(4-((S)-3-(tert-butoxy)-2-((S)-5-(tert-butoxy)-2-(4-iodobenzamido)-5-oxopentanamido)-3-oxopropyl)phenoxy)acetamido)-3-phenylpropanamido)-1-oxohexan-2-yl)carbamoyl)-L-glutamate (24).

Compound 24 was prepared from 20 (67 mg, 0.06 mmol), DIPEA (24 mg, 0.19 mmol) and 8 (33 mg, 0.096 mmol), following the same procedure described for compound 13. Compound 24: 41.4 mg (yield: 50.6%). ¹HNMR(400 MHz, CD₂Cl₂) δ: 7.76(d, 2H, J=8.0 Hz), 7.52(d, 2H, J=7.6 Hz), 7.22-7.32(m, 5H), 6.91(d, 2H, J=7.6 Hz), 6.57(d, 2H, J=7.2 Hz), 5.10-5.18(m, 2H), 4.73(s, 1H), 4.44(s, 1H), 4.21(s, 1H), 4.07(s, 1H), 3.13-3.34(m, 5H), 2.93-3.05(m, 2H), 2.25-2.48(m, 5H), 2.00-2.13 (m, 3H), 1.84-1.90(m, 2H), 1.32-1.49(m, 49H); HRMS calcd for C₆₄H₉₂IN₆O₁₆ (M+H)⁺, 1327.5614; found 1327.5533.

Di-tert-butyl (((S)-1-(tert-butoxy)-6-((S)-2-(2-(4-((S)-3-(tert-butoxy)-2-((S)-5-(tert-butoxy)-2-((S)-5-(tert-butoxy)-2-(4-iodobenzamido)-5-oxopentanamido)-5-oxopentanamido)-3-oxopropyl)phenoxy)acetamido)-3-phenylpropanamido)-1-oxohexan-2-yl)carbamoyl)-L-glutamate (25).

Compound 25 was prepared from 21 (50 mg, 0.04 mmol), DIPEA (6 mg, 0.048 mmol) and 8 (23 mg, 0.04 mmol), following the same procedure described for compound 13. Compound 25: 12.5 mg (yield: 18.6%). ¹HNMR(400 MHz, CDCl₃) δ: 7.85(d, 2H, J=8.4 Hz), 7.64-7.70(m, 3H), 7.17-7.26(m, 5H), 6.98-7.09(m, 3H), 6.72(d, 2H, J=7.6 Hz), 6.28(s, 1H), 5.70(s, 1H), 4.93-4.95(m, 1H), 4.66-4.67(m, 1H), 4.57-4.58(m, 2H), 4.14-4.37(m, 5H), 3.48-3.63(m, 1H), 3.35-3.38(m, 1H), 3.02-3.13(m, 5H), 2.40-2.52(m, 2H), 2.26-2.36(m, 6H), 1.85-2.16(m, 6H), 1.59-1.69(m, 2H), 1.41-1.50(m, 58H); HRMS calcd for C₇₃H₁₀₇IN₇O₁₉ (M+H)⁺, 1535.6564; found 1535.6607.

(((S)-1-Carboxy-5-((S)-2-(2-(4-((S)-2-carboxy-2-((S)-4-carboxy-2-(4-iodobenzamido)butanamido)ethyl)phenoxy)acetamido)-3-phenylpropanamido)pentyl)carbamoyl)-L-glutamic acid (26).

Compound 26 was prepared from 24 (41 mg, 0.03 mmol) in 1 mL TFA, following the same procedure described for compound 4. Compound 26: 16.0 mg (yield: 49.4%). ¹HNMR(400 MHz, MeOD) δ: 7.82(d, 2H, J=7.2 Hz), 7.55(d, 2H, J=7.6 Hz), 7.13-7.25(m, 7H), 6.74(d, 2H, J=7.6 Hz), 4.56-4.67(m, 3H), 4.23-4.42 (m, 4H), 3.58-3.63 (m, 2H), 2.93-3.19(m, 7H), 2.39-2.43 (m, 4H), 2.11-2.16(m, 2H), 1.99-2.06(m, 1H), 1.78-1.91(m, 2H), 1.60-1.65(m, 1H), 1.27-1.45(m, 4H); HRMS calcd for C₄₄H₅₂IN₆O₁₆ (M+H)⁺, 1047.2484; found 1047.2558.

(((S)-1-Carboxy-5-((S)-2-(2-(4-((S)-2-carboxy-2-((S)-4-carboxy-2-((S)-4-carboxy-2-(4- iodobenzamido)butanamido)butanamido)ethyl)phenoxy)acetamido)-3-phenylpropanamido)pentyl)carbamoyl)-L-glutamic acid (27).

Compound 27 was prepared from 25 (29 mg, 0.019 mmol) in 1 mL TFA, following the same procedure described for compound 4. Compound 27: 9.7 mg (yield: 41.4%). ¹HNMR(400 MHz, MeOD) δ: 8.15(d, 1H, J=8.4 Hz), 7.84(d, 2H, J=8.4 Hz), 7.61(d, 2H, J=8.4 Hz), 7.14-7.28(m, 7H), 6.82(d, 2H, J=8.4 Hz), 4.62-4.68(m, 2H), 4.39-4.55(m, 4H), 4.31-4.32(m, 1H), 4.23-4.24(m, 1H), 3.06-3.20(m, 4H), 2.92-3.02(m, 2H), 2.33-2.45(m, 6H), 2.03-2.15(m, 4H), 1.86-1.93(m, 2H), 1.74-1.78(m, 1H), 1.59-1.61(m, 1H), 1.36-1.44(m, 2H), 1.31-1.33(m, 2H); HRMS calcd for C₇₃H₁₀₇IN₇O₁₉ (M+H)⁺, 1535.6564; found 1535.6607.

Preparation of compound 29 was based on the following chemical reactions (Scheme 13)

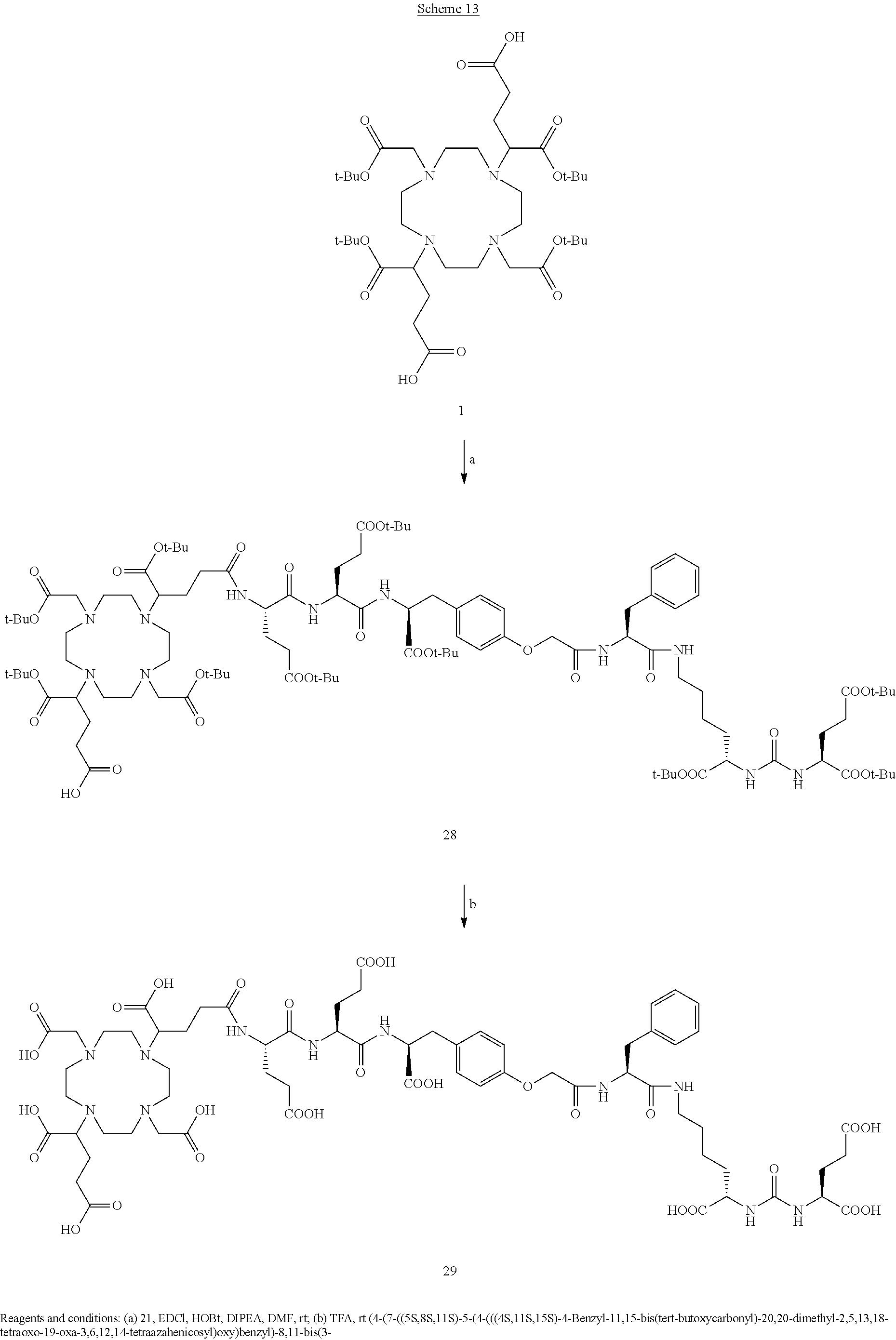

(tert-butoxy)-3-oxopropyl)-2,2,19,19-tetramethyl-4,7,10,13,17-pentaoxo-3,18-dioxa-6,9,12-triazaicosan-16-yl)-4,10-bis(2-(tert-butoxy)-2-oxoethyl)-1,4,7,10-tetraazacyclododecan-1-yl)-5-(tert-butoxy)-5-oxopentanoic acid (28).

To a solution of 21 (61 mg, 0.05 mmol) in 3 mL DMF, DIPEA (39 mg, 0.03 mmol), HOBt (17 mg, 0.1 mmol), EDC (19 mg, 0.1 mmol) and 1 (77 mg, 0.1 mmol) were added at 0° C. After stirred at rt overnight, 20 mL EtOAc were added to the reaction mixture. It was then washed with H₂O (10 mL×2) and brine (10 mL), dried over MgSO₄, and filtered. The filtrate was concentrated, and the residue was purified by FC (DCM/MeOH/NH₄OH=90/9/1) to give 25 mg 28 as colorless oil. (yield: 24.6%). HRMS calcd for C₁₀₄H₁₀₇N₁O₂₉ (M+H)⁺, 2037.2166; found 2037.2224.

(((1S)-5-((2S)-2-(2-(4-((2S)-2-((2S)-2-((2S)-2-(4-(4,10-Bis(carboxymethyl)-7-(1,3- dicarboxypropyl)-1,4,7,10-tetraazacyclododecan-1-yl)-4-carboxybutanamido)-4-carboxybutanamido)-4-carboxybutanamido)-2-carboxyethyl)phenoxy)acetamido)-3-phenylpropanamido)-1-carboxypentyl)carbamoyl)-L-glutamic acid (29).

Compound 29 was prepared from 28 (23 mg, 0.011 mmol) in 1 mL TFA, following the same procedure described for compound 4. Compound 29: 9.7 mg (yield: 59.8%). ¹HNMR(400 MHz, DMSO) δ: 8.15(s, 1H), 8.02-8.05(m, 3H), 7.18-7.25(m, 5H), 6.74(d, 2H, J=7.6 Hz), 6.28-6.33(m, 2H), 4.51-4.54(m, 2H), 4.37-4.41(m, 3H), 4.25-4.29(m, 2H), 4.03-4.10(m, 3H), 3.80(s, 4H), 3.59-3.62(m, 4H), 2.88-3.09(m, 18H), 2.24-2.33(m, 8H), 1.86-1.93(m, 6H), 1.63-1.75(m, 6H), 1.48-1.52(m, 2H), 1.34-1.36(m, 2H), 1.22-1.26(m, 2H); HRMS calcd for C₆₄H₈₉N₁₁O₂₉ (M+H)⁺, 1476.5906; found 1476.5995.

Preparation of compound 38 was based on the following chemical reactions (Scheme 14)

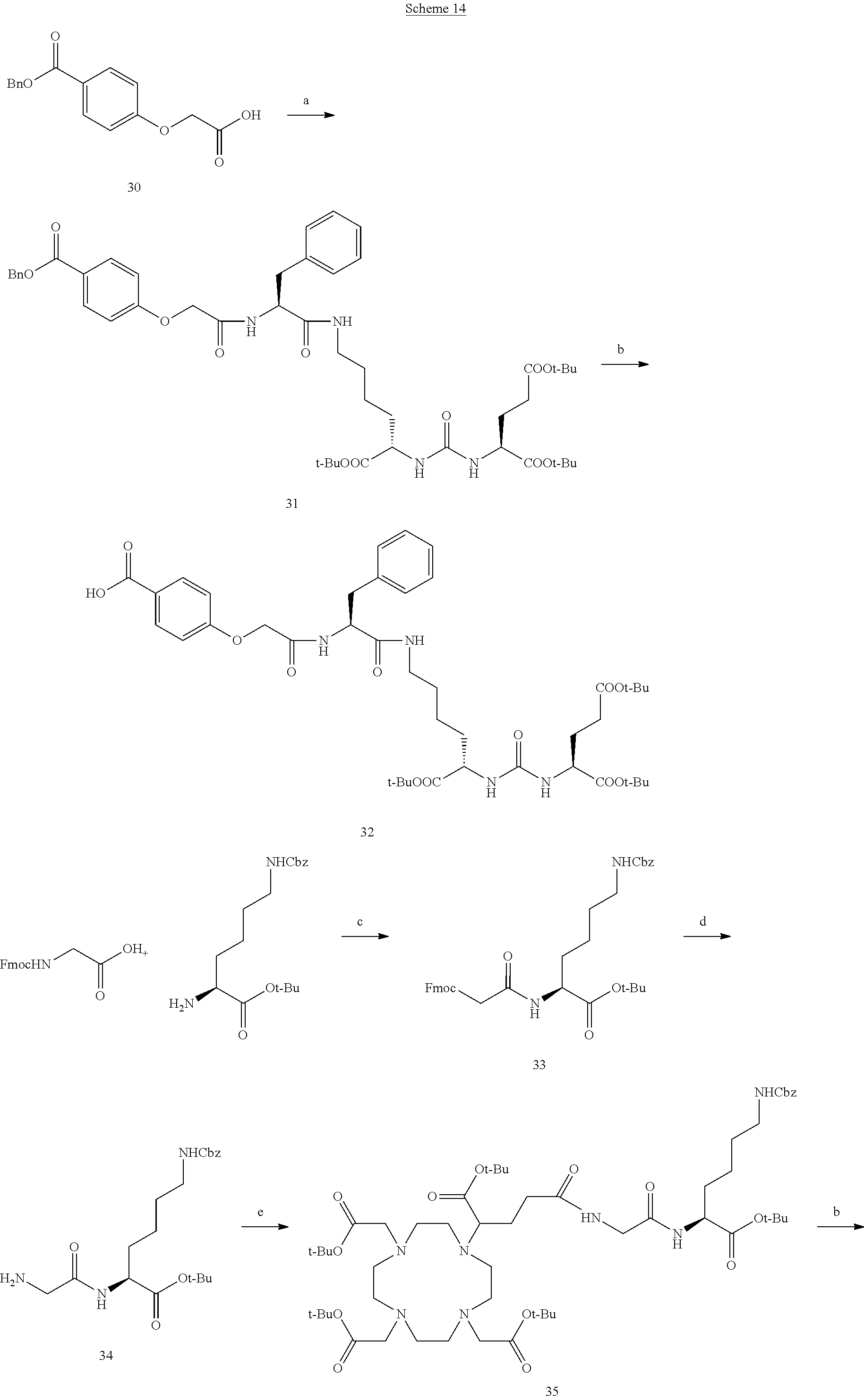

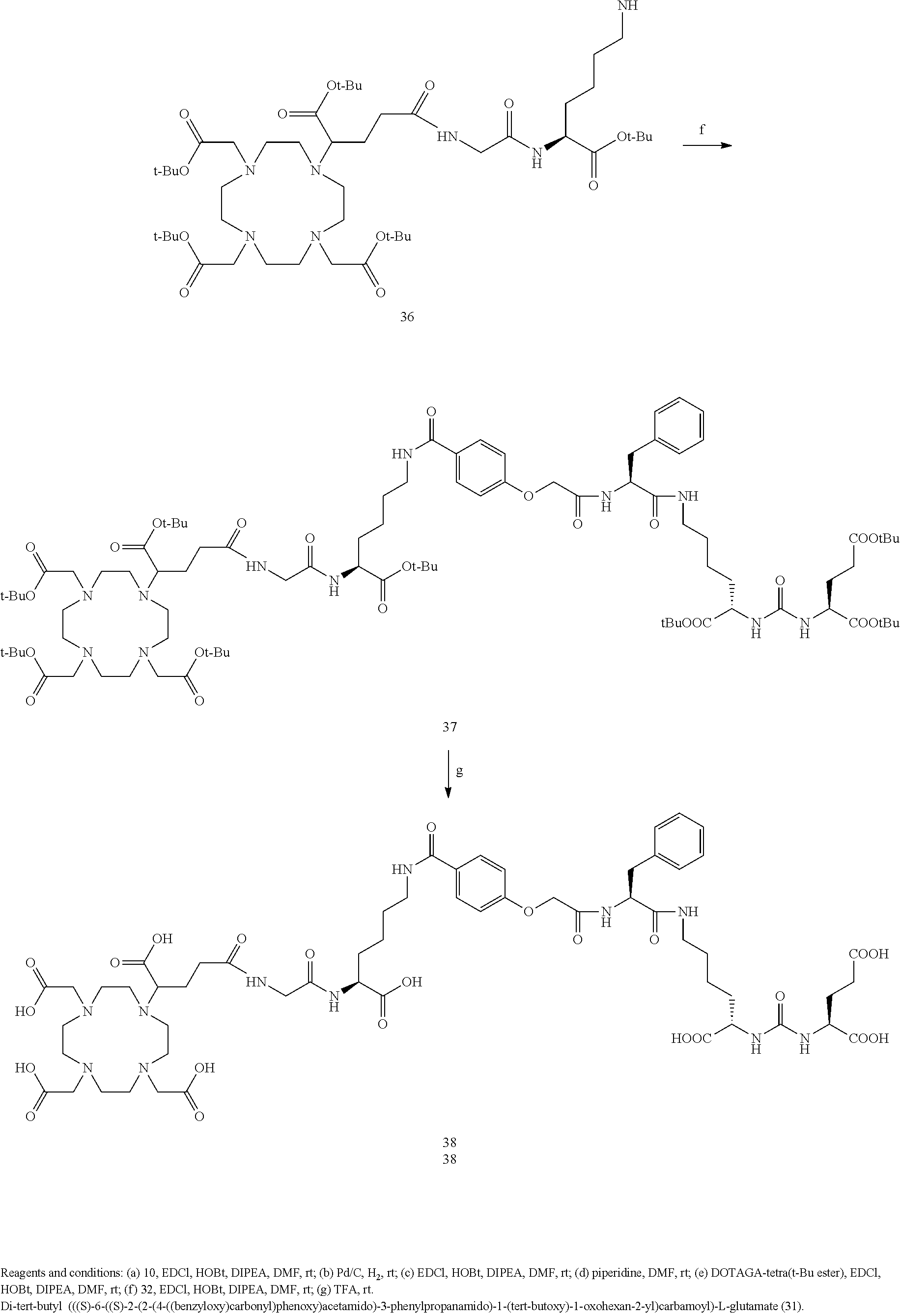

Compound 31 was prepared from 10 (635 mg, 1 mmol), DIPEA (387 mg, 3 mmol), HOBt(253 mg, 1.5 mmol), EDC(285 mg, 1.5 mmol) and 30 (286 mg, 1 mmol), following the same procedure described for compound 28. Compound 31: 672 mg (yield: 74.5%). HRMS calcd for C₄₉H₆₇N₄O₁₂ (M+H)⁺, 903.4755, found 903.4789.

4-(((4S,11S,15S)-4-Benzyl-11,15-bis(tert-butoxycarbonyl)-20,20-dimethyl-2,5,13,18-tetraoxo-19-oxa-3,6,12,14-tetraazahenicosyl)oxy)benzoic acid (32).

A mixture of the ester 31 (672 mg, 0.75 mmol) and 10% Pd/C (120 mg) in EtOH (20 mL) was shaken with hydrogen for 3 h. This mixture was then filtered and the filtrate was concentrated under vacuum to give 578 mg 32 as colorless oil (yield: 95%). HRMS calcd for C₄₂H₆₁N₄O₁₂ (M+H)⁺, 813.4286, found 813.4356.

tert-Butyl N⁶-((benzyloxy)carbonyl)-N²-glycyl-L-lysinate (34).

Compound 34 was prepared from H-Lys(Z)-OtBu (746 mg, 2 mmol), DIPEA (780 mg, 6 mmol), HOBt(506 mg, 3 mmol), EDC(570 mg, 3 mmol), piperidine (1 mL) and Fmoc-Gly-OH (594 mg, 2 mmol) following the same procedure described for compound 11. Compound 34: 424 mg (yield: 54.3%). HRMS calcd for C₂₀H₃₂N₃O₅ (M+H)⁺, 394.2342, found 394.2392.

Tri-tert-butyl 2,2′,2″-(10-((9S)-9-(tert-butoxycarbonyl)-20,20-dimethyl-3,11,14,18-tetraoxo-1-phenyl-2,19-dioxa-4,10,13 -triazahenicosan-17-yl)-1,4,7,10-tetraazacyclododecane-1,4,7-triyl)triacetate (35).

Compound 35 was prepared from DOTAGA-tetra(t-Bu ester) (140 mg, 0.2 mmol), DIPEA (78 mg, 0.6 mmol), HOBt(51 mg, 0.3 mmol), EDC(57 mg, 0.3 mmol) and 34 (79 mg, 0.2 mmol), following the same procedure described for compound 28. Compound 35: 103 mg (yield: 50.1%). HRMS calcd for C₅₅H₉₄N₇O₁₄(M+H)⁺, 1076.6859, found 1076.6938.

Tri-tert-butyl 2,2′,2″-(10-((5S)-5-(4-aminobutyl)-2,2,16,16-tetramethyl-4,7,10,14-tetraoxo-3,15-dioxa-6,9-diazaheptadecan-13-yl)-1,4,7,10-tetraazacyclododecane-1,4,7-triyl)triacetate (36).

Compound 36 was prepared from 35 (100 mg, 0.1 mmol), and Pd/C (20 mg), following the same procedure described for compound 32. Compound 36: 83.7 mg (yield: 89.0%). HRMS calcd for C₄₇H₈₈N₇O₁₂ (M+H)⁺, 942.6491, found 942.6583.

Di-tert-butyl (((2S)-1-(tert-butoxy)-6-((2S)-2-(2-(4-(((5S)-6-(tert-butoxy)-5-(2-(5-(tert-butoxy)-5-oxo-4-(4,7,10-tris(2-(tert-butoxy)-2-oxoethyl)-1,4,7,10-tetraazacyclododecan-1-yl)pentanamido)acetamido)-6-oxohexyl)carbamoyl)phenoxy)acetamido)-3-phenylpropanamido)-1-oxohexan-2-yl)carbamoyl)-L-glutamate (37).

Compound 37 was prepared from 36 (40 mg, 0.042 mmol), DIPEA (16.2 mg, 0.126 mmol), HOBt (11 mg, 0.063 mmol), EDC (12 mg, 0.063 mmol) and 32 (34 mg, 0.2 mmol), following the same procedure described for compound 28. Compound 37: 21 mg (yield: 28.8%). HRMS calcd for C₈₉H₁₄₆N₁₁O₂₃ (M+H)⁺, 1737.0593, found 1737.0675.

(((1S)-1-Carboxy-5-((2S)-2-(2-(4-(((5S)-5-carboxy-5-(2-(4-carboxy-4-(4,7,10-tris(carboxymethyl)-1,4,7,10-tetraazacyclododecan-1-yl)butanamido)acetamido)pentyl)carbamoyl)phenoxy)acetamido)-3-phenylpropanamido)pentyl)carbamoyl)-L-glutamic acid (38).

Compound 38 was prepared from 37 (20 mg, 0.011 mmol) in 1 mL TFA, following the same procedure described for compound 4. Compound 38: 6.8 mg (yield: 48.0%). HRMS calcd for C₅₇H₈₂N₁₁O₂₃ (M+H)⁺, 1288.5585; found 1476.5995.

Preparation of compound 42 was based on the following chemical reactions (Scheme 15)

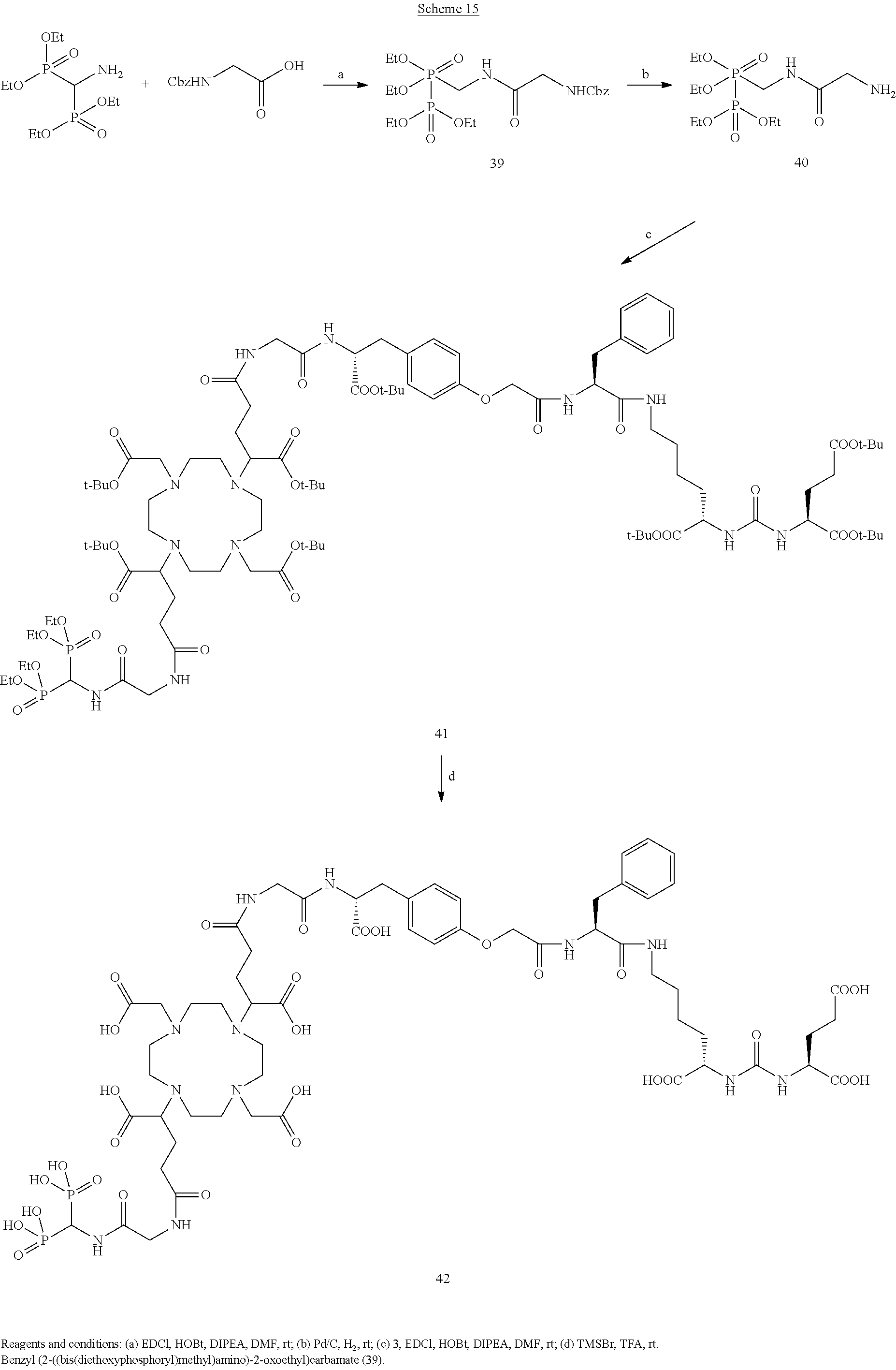

Compound 39 was prepared from Z-Gly (209 mg, 1 mmol), DIPEA (387 mg, 3 mmol), HOBt(253 mg, 1.5 mmol), EDC(285 mg, 1.5 mmol) and tetraethyl(aminomethylene)bis(phosphonate) (303 mg, 1 mmol) following the same procedure described for compound 28. Compound 39: 150 mg (yield: 30.4%). HRMS calcd for C₁₉H₃₃N₂O₉P₂(M+H)⁺, 495.1661, found 495.1679.

Tetraethyl ((2-aminoacetamido)methylene)bis(phosphonate) (40).

Compound 40 was prepared from 39 (1 g, 2 mmol), and Pd/C (200 mg), following the same procedure described for compound 32. Compound 40: 525 mg (yield: 72.9%). HRMS calcd for C₁₁H₂₇N₂O₇P₂(M+H)⁺, 361.1293, found 361.1342.

Di-tert-butyl (((2S)-6-((2S)-2-(2-(4-((2R)-2-(2-(4-(7-(5-((2-((bis(diethoxyphosphoryl)methyl)amino)-2-oxoethyl)amino)-1-(tert-butoxy)-1,5-dioxopentan-2-yl)-4,10-bis(2-(tert-butoxy)-2-oxoethyl)-1,4,7,10-tetraazacyclododecan-1-yl)-5-(tert-butoxy)-5-oxopentanamido)acetamido)-3-(tert-butoxy)-3-oxopropyl)phenoxy)acetamido)-3-(naphthalen-2-yl)propanamido)-1-(tert-butoxy)-1-oxohexan-2-yl)carbamoyl)-L-glutamate (41).

Compound 41 was prepared from 40 (13.7 mg, 0.038 mmol), DIPEA (14.7 mg, 0.114 mmol), HOBt (9.6 mg, 0.057 mmol), EDC (10.8 mg, 0.057 mmol) and 3 (65 mg, 0.038 mmol) following the same procedure described for compound 28. Compound 41: 44 mg (yield: 56.1%). HRMS calcd for C₉₉H₁₆₇N₁₂O₃₀P₂(M+H)⁺, 2066.1386, found 2066.1480.

(((1 S)-1-Carboxy-5-((2S)-2-(2-(4-((2R)-2-carboxy-2-(2-(4-carboxy-4-(7-(1-carboxy-4-((2-((diphosphonomethyl)amino)-2-oxoethyl)amino)-4-oxobutyl)-4,10-bis(carboxymethyl)-1,4,7,10-tetraazacyclododecan-1-yl)butanamido)acetamido)ethyl)phenoxy)acetamido)-3-phenylpropanamido)pentyl)carbamoyl)-L-glutamic acid (42).

To a solution of 41 (42 mg, 0.02 mmol) in 1 mL DMF was added 1 mL TMSBr at 0° C. The mixture was slowly warm to rt and stirred overnight and the solvent was removed in vacuo. The residue was treated with 1 mL TFA. After stirred at rt for 5 h, the solvent was removed and the residue was purified by semi-prep HPLC to give 12 mg 42 as white solid. (yield: 39.9%). ¹HNMR(400 MHz, DMSO) δ: 7.16-7.24(m, 5H), 7.09(d, 2H, J=8.4 Hz), 6.73(d, 2H, J=8.4 Hz), 4.49-4.53(m, 1H), 4.36-4.42(m, 4H), 4.07-4.10(m, 1H), 4.00-4.04(m, 1H), 3.68-3.83(m, 8H), 3.29-3.39(m, 2H), 3.17-3.28(m, 2H), 2.94-3.09(m, 12H), 2.79-2.88(m, 6H), 2.22-2.34(m, 6H), 1.88-1.94(m, 2H), 1.64-1.74(m, 2H), 1.49-1.54(m, 1H), 1.32-1.36(m, 2H), 1.17-1.24(m, 2H); HRMS calcd for C₅₉H₈₈N₁₂O₃₀P₂(M+2H)²⁺, 753.2597, found 753.2769.

Preparation of compound 51 was based on the following chemical reactions (Scheme 16)

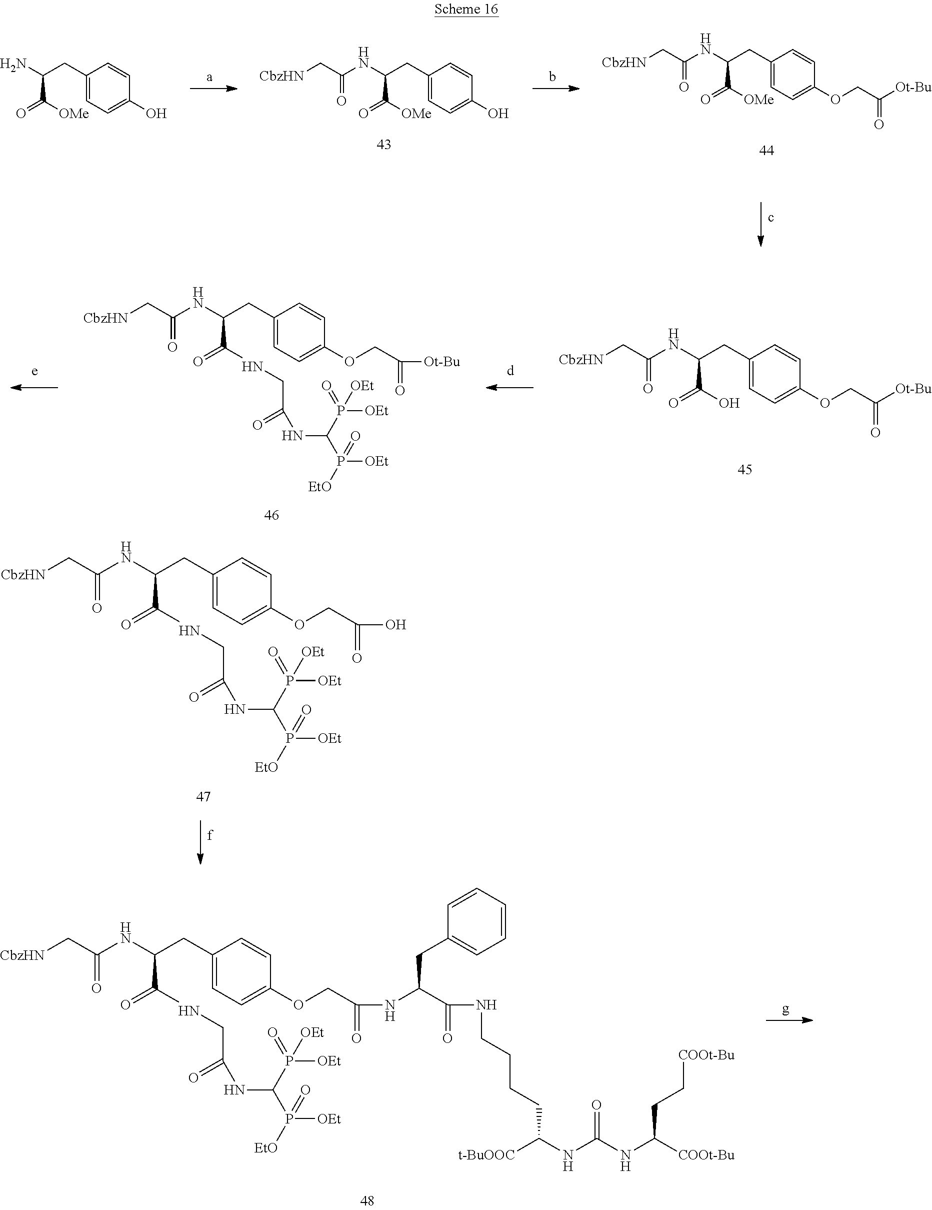

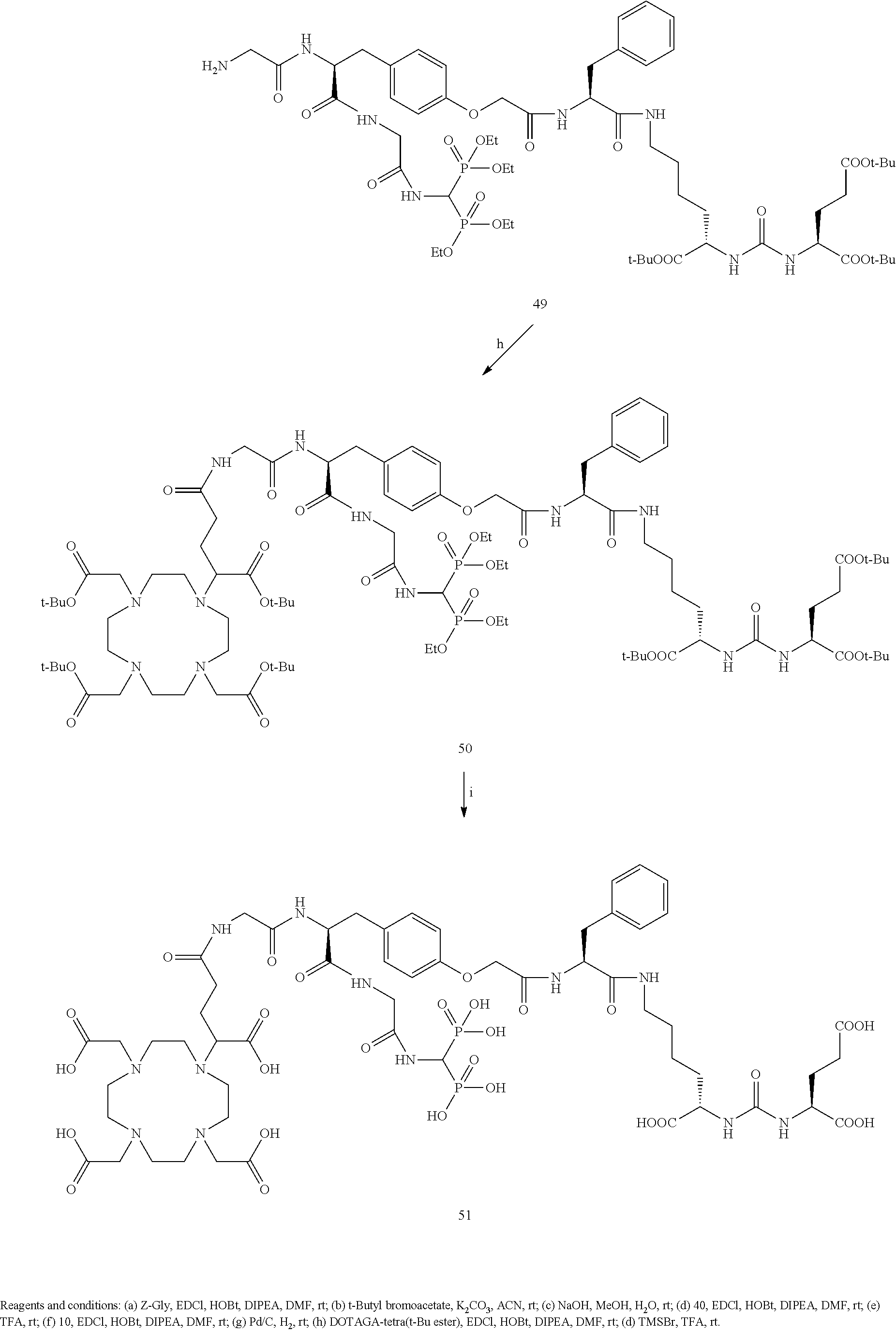

Methyl ((benzyloxy)carbonyl)glycyl-L-tyrosinate (43).

Compound 43 was prepared from Z-Gly (1.045 g, 5 mmol), DIPEA (1.94 g, 15 mmol), HOBt(1.26 g, 7.5 mmol), EDC(1.42 g, 7.5 mmol) and methyl L-tyrosinate (975 mg, 5 mmol) following the same procedure described for compound 28. Compound 43: 760 mg (yield: 50.5%). HRMS calcd for C₂₀H₂₃N₂O₆ (M+H)⁺, 387.1556, found 387.1579.

Methyl (S)-2-(2-(((benzyloxy)carbonyl)amino)acetamido)-3-(4-(2-(tert-butoxy)-2-oxoethoxy)phenyl)propanoate (44).

To a solution of 43 (760 mg, 2 mmol) in 20 mL ACN, t-butyl bromoacetate (390 mg, 2 mmol) and K₂CO₃ (552 mg, 4 mmol) were added. The mixture was then stirred at rt for 3 h and filtered. The filtrate was concentrated, and the residue was purified by FC (EtOAc/hexane=1/1) to give 44 as a colorless oil (yield: 770 mg, 77%). HRMS calcd. for C₂₆H₃₃N₂O₈ (M+H)⁺: 501.2237, found 501.2143.

(S)-2-(2-(((Benzyloxy)carbonyl)amino)acetamido)-3-(4-(2-(tert-butoxy)-2-oxoethoxy)phenyl)propanoic acid (45).

A solution of 44 (770 mg, 1.54 mmol) in 20 mL MeOH/NaOH (1 N) (1/1) was stirred at rt for 2 h. HCl (1 N) was then added to the reaction mixture to pH=4-5. The resulting mixture was extracted with EtOAc (50 mL×3). The organic layer was then dried over MgSO₄ and filtered. The filtrate was concentrated, and the residue was purified by FC (DCM/MeOH/NH₄OH=90/9/1) to give 45 as a white solid (yield: 560 mg, 74.8%). HRMS calcd. for C₂₅H₃₁N₂O₈ (M+H)⁺: 487.2080, found 487.1997. tert-Butyl (S)-2-(4-(2-(2-(((benzyloxy)carbonyl)amino)acetamido)-3-((2-((bis(diethoxyphosphoryl)methyl)amino)-2-oxoethyl)amino)-3-oxopropyl)phenoxy)acetate (46).

Compound 46 was prepared from 45 (560 mg, 1.15 mmol), DIPEA (451 mg, 3.5 mmol), HOBt(291 mg, 1.73 mmol), EDC(328 mg, 1.73 mmol) and 40 (400 mg, 1.11 mmol) following the same procedure described for compound 28. Compound 46: 760 mg (yield: 79.8%). HRMS calcd for C₃₆H₅₅N₄O₁₄P₂ (M+H)⁺, 829.3190, found 829.3320.

(S)-2-(4-(2-(2-(Benzyloxy)carbonyl)amino)acetamido)-3-((2-((bis(diethoxyphosphoryl)methyl)amino)-2-oxoethyl)amino)-3-oxopropyl)phenoxy)acetic acid (47).

A solution of 46 (760 mg, 0.92 mmol) in 10 mL TFA was tstirred at rt for 5 h. The solvent was removed, and the residue was purified by FC (EtOAc) to give 47 as a colorless oil (yield: 320 mg, 45.1%). HRMS calcd. for C₃₂H₄₇N₄O₁₄P₂(M+H)⁺: 773.2564, found 773.2652.

Di-tert-butyl (((S)-6-(S)-2-(2-(4-(S)-2-(2-(((benzyloxy)carbonyl)amino)acetamido)-3-((2-((bis(diethoxyphosphoryl)methyl)amino)-2-oxoethyl)amino)-3-oxopropyl)phenoxy)acetamido)-3-phenylpropanamido)-1-(tert-butoxy)-1-oxohexan-2-yl)carbamoyl)-L-glutamate (48).

Compound 48 was prepared from 47 (320 mg, 0.415 mmol), DIPEA (155 mg, 1.2 mmol), HOBt(100 mg, 0.6 mmol), EDC(114 mg, 0.6 mmol) and 10 (261 mg, 0.415 mmol) following the same procedure described for compound 28. Compound 48: 310 mg (yield: 53.8%). HRMS calcd for C₆₅H₉₉N₈O₂₁P₂ (M+H)⁺, 1389.6400, found 1389.6318. Di-tert-butyl (((S)-6-(S)-2-(2-(4-(S)-2-(2-aminoacetamido)-3-((bis(diethoxyphosphoryl)methyl)amino)-2-oxoethyl)amino)-3-oxopropyl)phenoxy)acetamido)-3-phenylpropanamido)-1-(tert-butoxy)-1-oxohexan-2-yl)carbamoyl)-L-glutamate (49).

Compound 49 was prepared from 48 (310 mg, 0.22 mmol), and Pd/C (60 mg), following the same procedure described for compound 32. Compound 49: 250 mg (yield: 90.6%). HRMS calcd for C₅₇H₉₃N₈O₁₉P₂ (M+H)⁺, 1255.6032, found 1255.6122. Di-tert-butyl (((2S)-6-((2S)-2-(2-(4-((2S)-3-((2-((bis(diethoxyphosphoryl)methyl)amino)-2-oxoethyl)amino)-2-(2-(5-(tert-butoxy)-5-oxo-4-(4,7,10-tris(2-(tert-butoxy)-2-oxoethyl)-1,4,7,10-tetraazacyclododecan-1-yl)pentanamido)acetamido)-3-oxopropyl)phenoxy)acetamido)-3-phenylpropanamido)-1-(tert-butoxy)-1-oxohexan-2-yl)carbamoyl)-L-glutamate (50).

Compound 50 was prepared from 49 (230 mg, 0.183 mmol), DIPEA (58 mg, 0.45 mmol), HOBt(38 mg, 0.225 mmol), EDC(43 mg, 0.225 mmol) and DOTAGA-tetra(t-Bu ester) (107 mg, 0.152 mmol) following the same procedure described for compound 28. Compound 50: 58 mg (yield: 19.7%). HRMS calcd for C₉₂H₁₅₅N₁₂O₂₈P₂ (M+H)⁺, 1938.0549, found 1938.0721.

(((1S)-1-Carboxy-5-((2S)-2-(2-(4-((2S)-2-(2-(4-carboxy-4-(4,7,10-tris(carboxymethyl)-1,4,7,10-tetraazacyclododecan-1-yl)butanamido)acetamido)-3-((2-((diphosphonomethyl)amino)-2-oxoethyl)amino)-3-oxopropyl)phenoxy)acetamido)-3-phenylpropanamido)pentyl)carbamoyl)-L-glutamic acid (51).

Compound 51 was prepared from 50 (50 mg, 0.026 mmol), TMSBr (1 mL) , DMF (1 mL) and TFA (1 mL), following the same procedure described for compound 42. Compound 51: 12 mg (yield: 32.2%). ¹HNMR(400 MHz, DMSO) δ: 7.13-7.27(m, 5H), 6.74(d, 2H, J=8.4 Hz), 6.28-6.34(m, 3H), 4.45-4.57(m, 5H), 4.04-4.11(m, 2H), 3.74-3.94(m, 6H), 3.48-3.61(m, 6H), 3.30-3.32(m, 2H), 2.84-3.10(m, 12H), 2.72-2.74(m, 2H), 2.45-2.47(m, 4H), 2.22-2.28(m, 2H), 1.88-1.97(m, 2H), 1.64-1.74(m, 2H), 1.49-1.53(m, 1H), 1.33-1.38(m, 2H), 1.25-1.29(m, 2H); HRMS calcd for C₅₆H₈₁N₁₂O₂₈P₂ (M−H)⁻, 1431.4764; found 1431.4543.

(4S,11S,15S)-4-benzyl-1-(4-((2S)-2-(2-(4-(4,10-bis(carboxymethyl)-7-(1,3-dicarboxypropyl)-1,4,7,10-tetraazacyclododecan-1-yl)-4-carboxylatebutanamido)acetamido)-2-carboxyethyl)phenoxy)-2,5,13-trioxo-3,6,12,14-tetraazaheptadecane-11,15,17-tricarboxylate gallium ([^{nat}Ga]4).

To a solution of compound 4 (30 mg, 0.0235 mmol) in 1 mL H2O was added 60 μL GaCl3 solution (1.13 M). The pH was adjusted to 4-5 by adding 1 N HCl and the mixture was stirred at 80° C. for 1 h and then purified by semi-prep HPLC. Solvent was removed under vacuum to give 6.8 mg white solid. HRMS calculated for C₅₆H₇₆GaN₁₀O₂₄ (M+H)+: 1341.4290, found 1341.4325.

(4S,11S,15 S)-4-benzyl-1-(4-(2S)-2-(2-(4-(4,10-bis(carboxymethyl)-7-(1,3-dicarboxypropyl)-1,4,7,10-tetraazacyclododecan-1-yl)-4-carboxylatebutanamido)acetamido)-2-carboxyethyl)phenoxy)-2,5,13-trioxo-3,6,12,14-tetraazaheptadecane-11,15,17-tricarboxylate lutetium ([^{nat}Lu]4).

A solution of LuCl3 (0.25 M) in 100 μL 0.1 N HCl was added to the compound 4 (20 mg, 15.7 μmol) in 1 mL HEPES (0.5 M, pH 5). The mixture was stirred at 98° C. for 10 min and then purified by semi-prep HPLC. Solvent was removed under vacuum to give 15 mg white solid. HRMS calculated for C₅₆H₇₆LuN₁₀O₂₄ (M+H)+: 1487.4442, found 1487.4527.

### EXAMPLE 6

### Evaluation of PSMA Binding Affinity—IC50

In vitro binding assays were carried out to determine the PSMA binding affinity of various compounds. By incubating PSMA positive cells either with: 1). LNCaP with 0.2 nM [⁶⁸Ga]PSMA-11 or [¹²⁵I]MIP-1095, as the ligand, in the presence of 10 different concentrations of competing ligands; non-specific binding was defined with 20 μM 2-PMPA (2-(phosphonomethyl)pentanedioic acid); or 2). PC-3 PIP cells with [¹²⁵I]MIP-1095 (0.18 nM diluted in PBS) in presence of different concentration of test compounds (10⁻⁵-10⁻¹⁰ nM diluted in PBS containing 0.1% bovine serum albumin). Nonspecific binding (NSB) was defined with 2 μM the known PSMA inhibitor, PSMA-617. After incubation at 37° C. for 1 h, the bound and free fractions were separated by vacuum filtration through GF/B filters using a Brandel M-24R cell harvester. The filters were washed twice with cold Tris-HCl buffer (50 mM, pH=7.4), and the radioactivity on the filters was counted in a gamma counter (Wizard², Perkin-Elmer) with 50% efficiency. The nonspecific bound was less than 10% of the total bound. Data were analyzed using GraphPad Prism 6.0 with a nonlinear regression algorithm to obtain the half maximal inhibitory concentration (IC₅₀).

The binding affinities to PSMA of the test compounds were measured by a competitive binding assay using either LNCap or PC-3 PIP cells suspension and the known radiotracers known to have high affinity and specificity to PSMA, [⁶⁸Ga]PSMA-11 or [¹²⁵I]MIP-1095. The IC₅₀ values of four iodinated compounds, three DOTA, DOTAG and DOTA(GA)2 related compounds and two known PSMA inhibitors are shown in Table 1. Complexes of compound 4 and natural Ga and natural Lu were also tested. Results showed that all of the compounds claimed in this application displayed excellent binding affinity showing IC50 values between 1 to 50 nM. After labeling with radioisotope they are expected to bind to the tumor tissues over expressing PSMA binding sites.

TABLE 1
Binding affinity to PSMA binding sites (IC50, nM, n = 3)

| | | IC₅₀ (LNCaP human | |
|---|---|---|---|
| | | PCa cells and | IC₅₀ *(PC-3 PIP cells |
| ID number | Structure | [68Ga]PSMA-11) | and [125I]MIP-1095) |
| PSMA-11 | 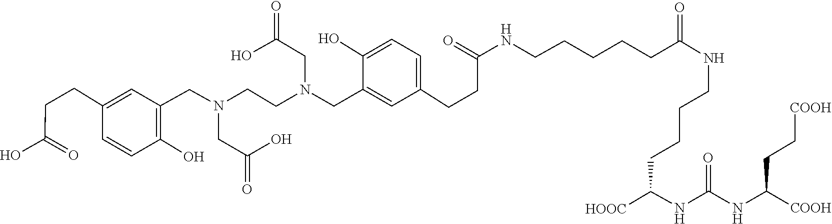 [SMILES: O=C(O)CCc1ccc(O)c(CN(CCN(CC(=O)O)Cc2cc(CCC(=O)NCCCCCC(=O)NCCCC[C@H](NC(=O)N[C@@H](CCC(=O)O)C(=O)O)C(=O)O)ccc2O)CC(=O)O)c1] | | 47.8 ± 3.16 |
| PSMA-617 | 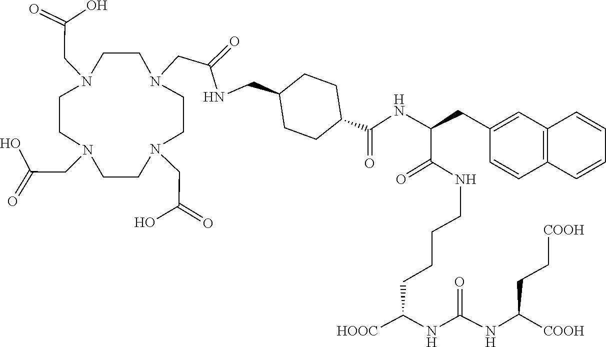 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O] | 11.1 ± 0.8 | 10.9 ± 0.37 |
| 4 | 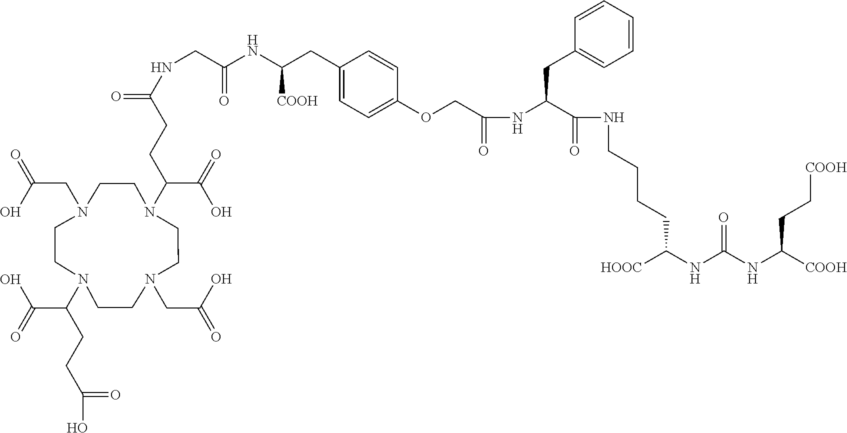 [SMILES: O=C(O)CCC(C(=O)O)N1CCN(CC(=O)O)CCN(C(CCC(=O)NCC(=O)N[C@@H](Cc2ccc(OCC(=O)N[C@@H](Cc3ccccc3)C(=O)NCCCC[C@H](NC(=O)N[C@@H](CCC(=O)O)C(=O)O)C(=O)O)cc2)C(=O)O)C(=O)O)CCN(CC(=O)O)CC1] | 28.7 ± 1.7 | 33.9 ± 2.38 |
| 7 | 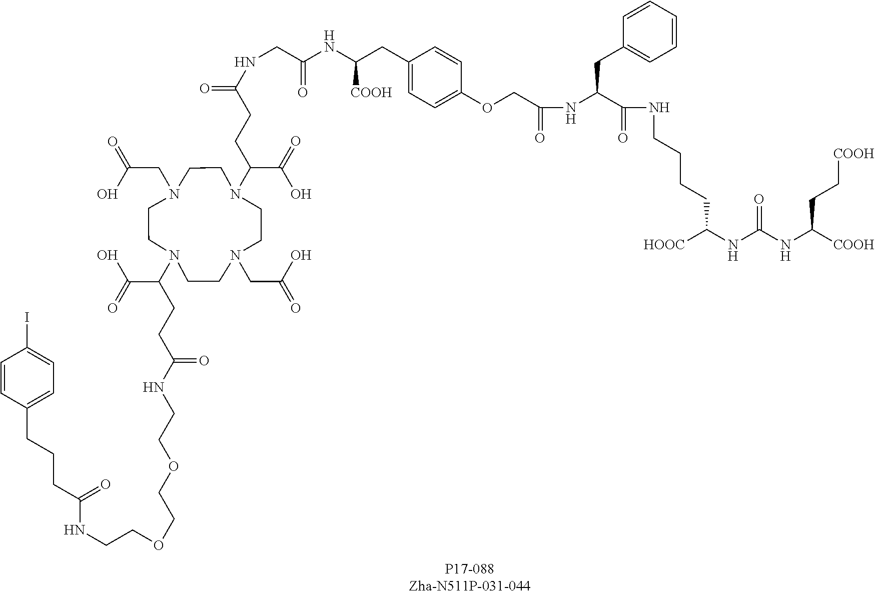 [SMILES: O=C(O)CC[C@H](NC(=O)N[C@@H](CCCCNC(=O)[C@H](Cc1ccccc1)NC(=O)COc1ccc(C[C@H](NC(=O)CNC(=O)CCC(C(=O)O)N2CCN(CC(=O)O)CCN(C(CCC(=O)NCCOCCOCCNC(=O)CCCc3ccc(I)cc3)C(=O)O)CCN(CC(=O)O)CC2)C(=O)O)cc1)C(=O)O)C(=O)O] | 15.4 ± 0.8 | 13.6 ± 1.02 |
| [^{nat}Ga]4 | 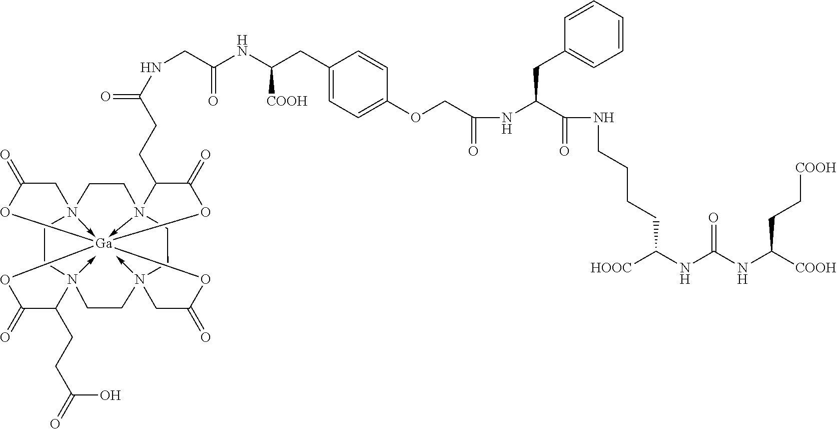 | | 22.2 ± 2.1^{a} |
| [^{nat}Lu]4 | 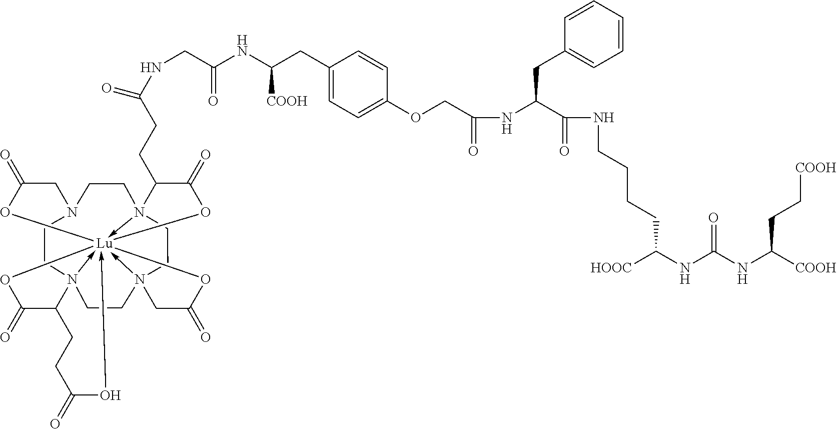 | | 18.7 ± 0.3^{a} |
| 17 | 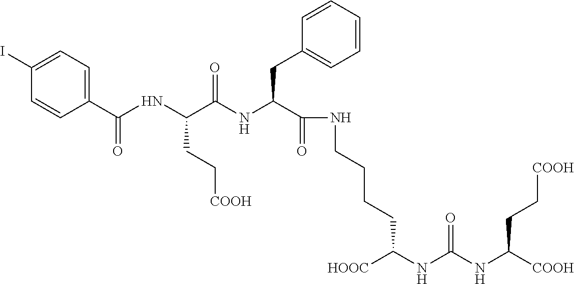 [SMILES: O=C(O)CC[C@H](NC(=O)N[C@@H](CCCCNC(=O)[C@H](Cc1ccccc1)NC(=O)[C@H](CCC(=O)O)NC(=O)c1ccc(I)cc1)C(=O)O)C(=O)O] | | 2.63 ± 0.13 |
| 18 | 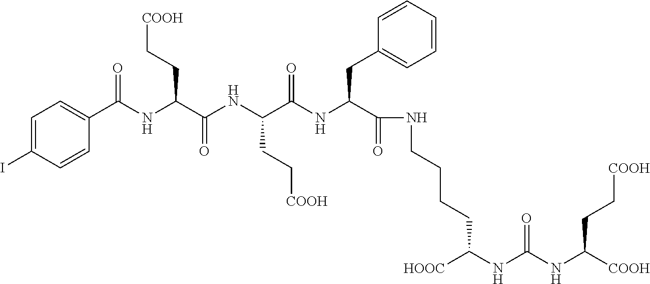 [SMILES: O=C(O)CC[C@H](NC(=O)N[C@@H](CCCCNC(=O)[C@H](Cc1ccccc1)NC(=O)[C@H](CCC(=O)O)NC(=O)[C@H](CCC(=O)O)NC(=O)c1ccc(I)cc1)C(=O)O)C(=O)O] | | 1.99 ± 0.06 |
| 26 | 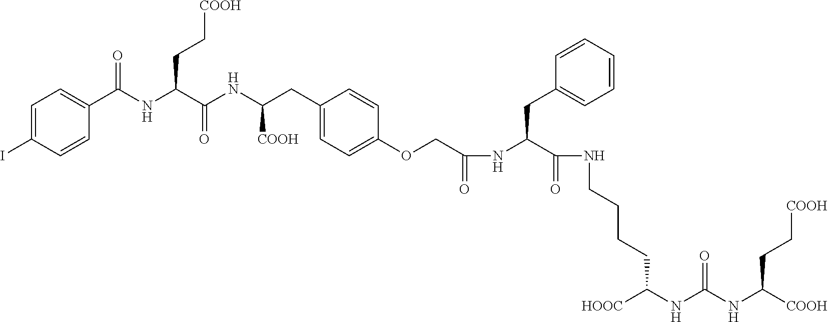 [SMILES: O=C(O)CC[C@H](NC(=O)N[C@@H](CCCCNC(=O)[C@H](Cc1ccccc1)NC(=O)COc1ccc(C[C@H](NC(=O)[C@H](CCC(=O)O)NC(=O)c2ccc(I)cc2)C(=O)O)cc1)C(=O)O)C(=O)O] | | 4.74 ± 0.37 |
| 27 | 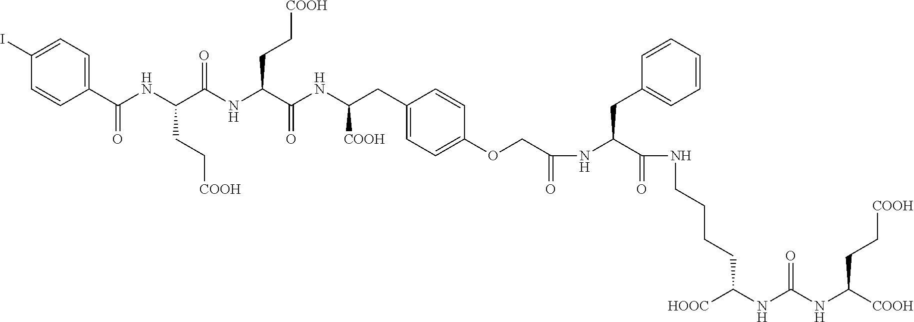 [SMILES: O=C(O)CC[C@H](NC(=O)N[C@@H](CCCCNC(=O)[C@H](Cc1ccccc1)NC(=O)COc1ccc(C[C@H](NC(=O)[C@H](CCC(=O)O)NC(=O)[C@H](CCC(=O)O)NC(=O)c2ccc(I)cc2)C(=O)O)cc1)C(=O)O)C(=O)O] | | 3.52 ± 0.19 |
| 29 | 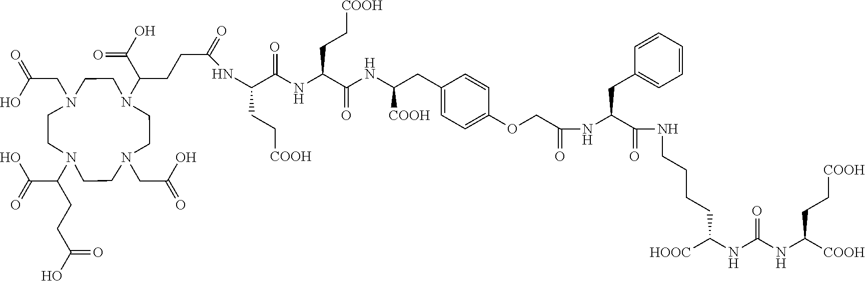 [SMILES: O=C(O)CCC(C(=O)O)N1CCN(CC(=O)O)CCN(C(CCC(=O)N[C@@H](CCC(=O)O)C(=O)N[C@@H](CCC(=O)O)C(=O)N[C@@H](Cc2ccc(OCC(=O)N[C@@H](Cc3ccccc3)C(=O)NCCCC[C@H](NC(=O)N[C@@H](CCC(=O)O)C(=O)O)C(=O)O)cc2)C(=O)O)C(=O)O)CCN(CC(=O)O)CC1] | | 22.6 ± 1.59 |
| 38 | 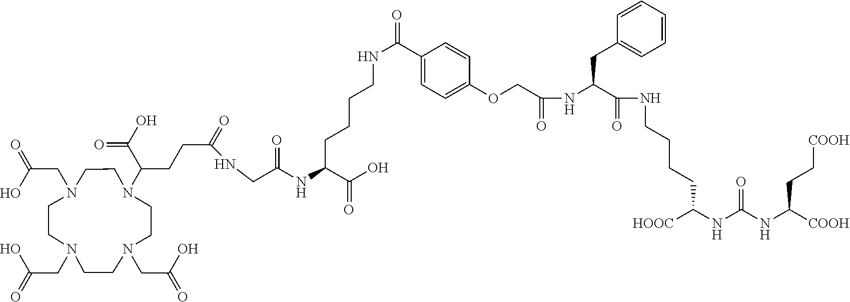 [SMILES: O=C(O)CC[C@H](NC(=O)N[C@@H](CCCCNC(=O)[C@H](Cc1ccccc1)NC(=O)COc1ccc(C(=O)NCCCC[C@H](NC(=O)CNC(=O)CCC(C(=O)O)N2CCN(CC(=O)O)CCN(CC(=O)O)CCN(CC(=O)O)CC2)C(=O)O)cc1)C(=O)O)C(=O)O] | | 28.1 ± 0.85 |
| 42 | 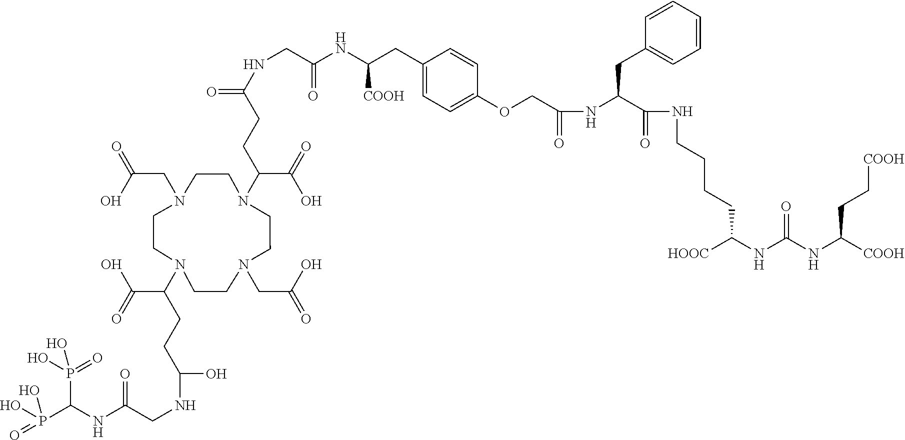 [SMILES: O=C(O)CC[C@H](NC(=O)N[C@@H](CCCCNC(=O)[C@H](Cc1ccccc1)NC(=O)COc1ccc(C[C@H](NC(=O)CNC(=O)CCC(C(=O)O)N2CCN(CC(=O)O)CCN(C(CCC(O)NCC(=O)NC(P(=O)(O)O)P(=O)(O)O)C(=O)O)CCN(CC(=O)O)CC2)C(=O)O)cc1)C(=O)O)C(=O)O] | 19.3 ± 1.9 | |
| 51 | 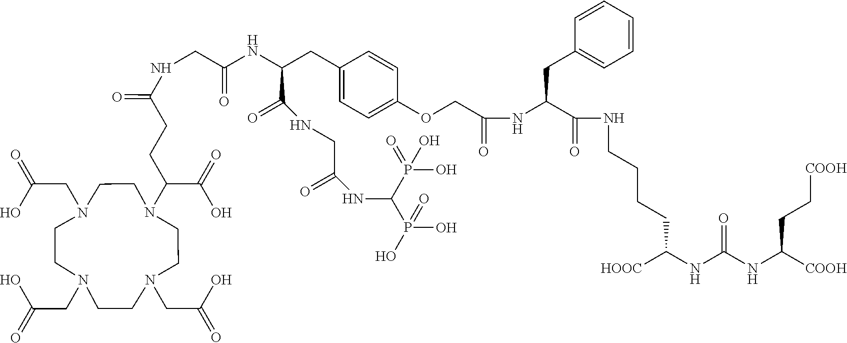 [SMILES: O=C(O)CC[C@H](NC(=O)N[C@@H](CCCCNC(=O)[C@H](Cc1ccccc1)NC(=O)COc1ccc(C[C@H](NC(=O)CNC(=O)CCC(C(=O)O)N2CCN(CC(=O)O)CCN(CC(=O)O)CCN(CC(=O)O)CC2)C(=O)NCC(=O)NC(P(=O)(O)O)P(=O)(O)O)cc1)C(=O)O)C(=O)O] | 10.7 ± 1.6 | |

^{a}: n = 2

### EXAMPLE 7

### In Vitro Cell Uptakes

To determine the cell uptakes of the [¹⁷⁷Lu] labelled ligands, 5×10⁵ cells/well were grown in 12-well plates in 1 mL of medium for 48 h. The cells were washed twice with PBS, and 900 μL fresh media were added. Radiolabelled ligand was added and the PSMA-inhibitor (2-PMPA) was applied in a final concentration of 10 μM to determine unspecific binding. All samples were prepared in triplicate. Following the incubation at 37° C., cells were washed twice to remove unbound activity and afterwards lysed in 1 mL of 0.5 M NaOH. Activity was measured in a gamma counter. Aliquots of the solution added to the cells were also measured for the calculation of the cellular uptake as % ID. All of the ¹⁷⁷Lu labeled ligands displayed high specific uptakes in the PSMA-positive cell line, PIP PC3. Especially, [¹⁷⁷Lu]4 and [¹⁷⁷Lu]51 showed much higher uptakes than those of reference ligand, [¹⁷⁷Lu]PSMA-617 suggesting the they might have superior PSMA binding and retention. No specific binding was observed for the PSMA-negative cell line, PC3.

TABLE 2
In vitro cell uptake studies (% ID per 5 × 10⁵ cells, Avg. n = 3)

| | Time | [¹⁷⁷Lu] | | | | |
|---|---|---|---|---|---|---|
| | (min) | PSMA-617 | [¹⁷⁷Lu]4 | [¹⁷⁷Lu]7 | [¹⁷⁷Lu]42 | [¹⁷⁷Lu]51 |
| PIP PC3 | 5 | 3.5 | 4.0 | 5.0 | 3.3 | 4.4 |
| PIP PC3 | 15 | 5.8 | 7.5 | 7.8 | 5.5 | 8.4 |
| PIP PC3 | 30 | 6.8 | 11.2 | 10.1 | 7.6 | 13.4 |
| PIP PC3 | 60 | 8.2 | 13.4 | 11.4 | 8.6 | 17.4 |
| PIP PC3 | 120 | 9.6 | 14.9 | 11.9 | 9.8 | 19.3 |
| PIP PC3* | 60 | 1.0 | 0.9 | 1.3 | 0.9 | 1.1 |
| PC3** | 60 | 0.3 | 0.4 | 0.7 | 0.8 | 0.7 |

*Assays were performed by incubating with the PSMA inhibitor (2-PMPA, 2 uM) which showed a complete inhibition of PSMA uptake.
**PC3 cells are normal tumor cells and they do not express PSMA.

### EXAMPLE 8

### Biodistribution of [⁶⁸Ga]4, ¹⁷⁷Lu labeled compounds 4 and 7 in tumor-bearing nude mice

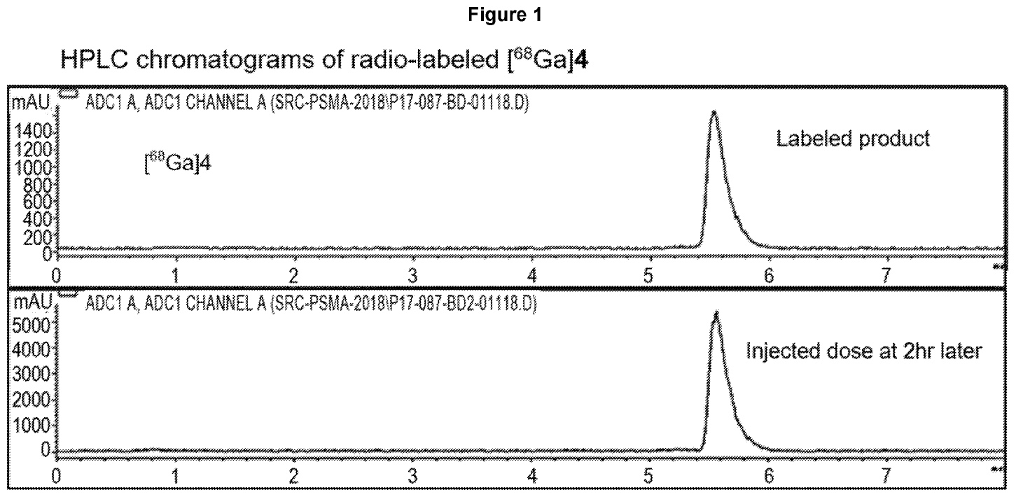

⁶⁸Ga labeling: To 15 nmole ligand 4 (1 mg/mL DMSO) were added 20 μL of 2.0 N NaOAc, 500 μL ⁶⁸Ga-solution (2.25 mCi). The reaction was heated in a heating block at 90° C. for 10 minutes in a 3 mL closed vial. After cooling, the sample was analyzed by HPLC (HPLC: Eclise XDB C18 150×4.6 mm, gradient, 2 mL/min; A: 0.1% TFA in water; B: 0.1% TFA in ACN: 0-2 min 100% A; 2-4 min: from 0% to 100% B; 4-9 min: 100% B; 9-10 min: from 100% to 0% B). Radiochemical purity of [⁶⁸Ga]4 was >99% RCP (FIG. 1) and injected doses were stable at 2 hr after formulation.

For iv injection 150 μL of labeled solution was diluted with saline to 3 mL. Mice were injected with 150 μL of formulated dose. Injected radioactivity was 19-28 μCi and PSMA ligand amount was constant at 0.2 nmole/mouse.

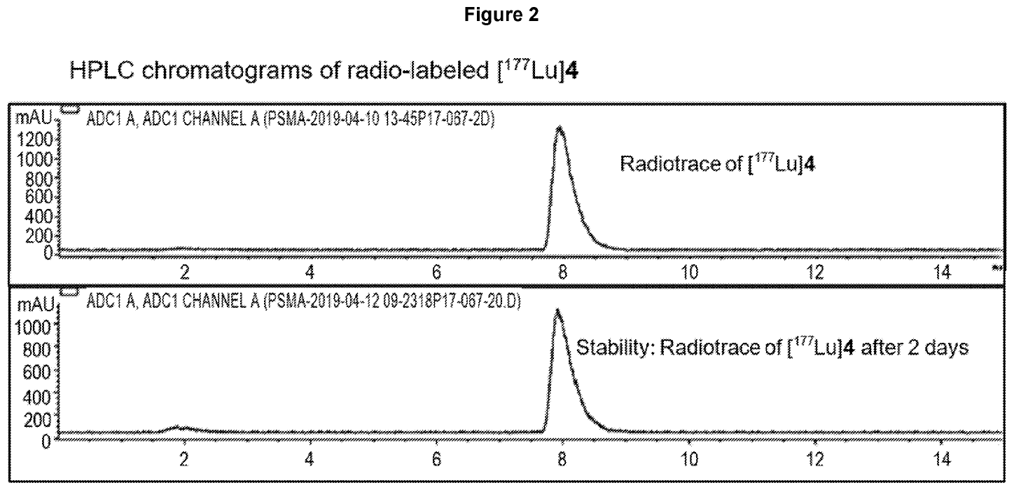

¹⁷⁷Lu labeling: To 10 μg ligand (1 mg/mL DMSO) were added 15 μL of 2.0 N NaOAc, 400 μL 0.05 N HCl, and 20 μL ¹⁷⁷Lu-solution (780 μCi (Capintec setting 450 (reading×10)). The reaction was heated with a heating block at 95° C. for 1 hour in a 3 mL closed vial. After cooling, the sample was analyzed by HPLC (HPLC: Eclipse XDB-C18 150×4.6 mm, gradient, 1 mL/min; A: 0.1% TFA in water; B: 0.1% TFA in ACN: 0-4 min A/B 85/15% ; 4-11 min: from 85/15 to 30/70%; 11-14 min: from 30/70% to 85/15%). Radiochemical purity of [¹⁷⁷Lu]4 (FIG. 2) and [¹⁷⁷Lu]7 was >98% and injected doses were stable at 48 hr after formulation.

For iv injection 150 μL of labeled solution was diluted with saline to 3.75mL. Mice were injected with 150 μL of formulated dose. Injected radioactivity was 100 μCi and PSMA ligand amount was constant at 0.72 nmole/mouse.

TABLE 3a
Biodistribution of [⁶⁸Ga]4 in tumor bearing nude mice
CD-1 male nude mice bearing PC3-PIP (PSMA
positive) and PC-3 tumors (PSMA negative)
[⁶⁸Ga]4 (% dose/g (Avg ± sd, n = 3))

| % dose/g | 30 min | 1 hr | 2 hr |
|---|---|---|---|
| Blood | 1.22 ± 0.06 | 0.65 ± 0.08 | 0.53 ± 0.11 |
| Heart | 0.75 ± 0.12 | 0.43 ± 0.06 | 0.25 ± 0.04 |
| Muscle | 0.51 ± 0.07 | 0.21 ± 0.05 | 0.14 ± 0.07 |
| Lung | 2.46 ± 0.37 | 1.52 ± 0.28 | 1.05 ± 0.08 |
| Kidney | 137.36 ± 12.92 | 166.31 ± 18.62 | 116.41 ± 51.94 |
| Spleen | 7.19 ± 1.55 | 4.60 ± 1.80 | 3.41 ± 2.46 |
| Pancreas | 1.57 ± 0.93 | 0.75 ± 0.13 | 0.50 ± 0.15 |
| Liver | 3.23 ± 0.31 | 2.87 ± 0.30 | 2.54 ± 0.16 |
| Skin | 1.71 ± 0.22 | 0.64 ± 0.26 | 0.62 ± 0.31 |
| Brain | 0.04 ± 0.01 | 0.03 ± 0.00 | 0.03 ± 0.00 |
| Bone | 0.35 ± 0.01 | 0.17 ± 0.01 | 0.19 ± 0.03 |
| Stomach | 0.44 ± 0.28 | 0.34 ± 0.19 | 0.20 ± 0.09 |
| Intestine | 0.48 ± 0.12 | 0.44 ± 0.13 | 0.38 ± 0.04 |
| PIP- | 12.88 ± 2.08 | 13.86 ± 1.54 | 16.74 ± 2.75 |
| PSMA+ | | | |
| tumor | | | |
| PC3- | 1.59 ± 0.27 | 1.14 ± 0.25 | 0.74 ± 0.23 |
| PSMA- | | | |
| tumor | | | |

TABLE 3b
Biodistribution of [¹⁷⁷Lu]4 in tumor bearing nude mice
CD-1 male nude mice bearing PC3-PIP (PSMA positive)
and PC-3 tumors (PSMA negative)
[¹⁷⁷Lu]4 (% dose/g (Avg ± SD of n = 4)

| % dose/g | | | 24 hrs | 48 hrs | 96 hrs | 192 hrs |
|---|---|---|---|---|---|---|
| (n = 4) | 1 hr | 4 hrs | (1 day) | (2 days) | (4 days) | (8 days) |
| Blood | 0.27 ± 0.07 | 0.02 ± 0.01 | 0.002 ± 0.000 | 0.003 ± 0.002 | 0.001 ± 0.000 | 0.001 ± 0.000 |
| Heart | 0.17 ± 0.03 | 0.11 ± 0.07 | 0.011 ± 0.002 | 0.010 ± 0.000 | 0.011 ± 0.006 | 0.005 ± 0.000 |
| Muscle | 0.18 ± 0.06 | 0.05 ± 0.01 | 0.012 ± 0.003 | 0.006 ± 0.003 | 0.006 ± 0.002 | 0.003 ± 0.003 |
| Lung | 0.63 ± 0.13 | 0.20 ± 0.05 | 0.024 ± 0.005 | 0.032 ± 0.028 | 0.020 ± 0.009 | 0.006 ± 0.003 |
| Kidney | 45.59 ± 20.51 | 21.17 ± 5.08 | 2.291 ± 0.761 | 2.359 ± 1.504 | 0.623 ± 0.072 | 0.177 ± 0.009 |
| Spleen | 1.42 ± 1.44 | 0.53 ± 0.26 | 0.047 ± 0.005 | 0.133 ± 0.173 | 0.033 ± 0.015 | 0.032 ± 0.010 |
| Pancreas | 0.43 ± 0.20 | 0.18 ± 0.10 | 0.015 ± 0.004 | 0.011 ± 0.001 | 0.022 ± 0.011 | 0.003 ± 0.001 |
| Liver | 0.18 ± 0.05 | 0.09 ± 0.03 | 0.036 ± 0.003 | 0.069 ± 0.067 | 0.031 ± 0.003 | 0.021 ± 0.005 |
| Skin | 0.35 ± 0.07 | 0.12 ± 0.05 | 0.059 ± 0.029 | 0.034 ± 0.015 | 0.032 ± 0.019 | 0.011 ± 0.003 |
| Brain | 0.02 ± 0.01 | 0.01 ± 0.00 | 0.006 ± 0.001 | 0.006 ± 0.002 | 0.006 ± 0.001 | 0.003 ± 0.001 |
| Bone | 0.15 ± 0.05 | 0.05 ± 0.01 | 0.022 ± 0.010 | 0.030 ± 0.010 | 0.043 ± 0.038 | 0.042 ± 0.035 |
| Stomach | 0.20 ± 0.10 | 0.12 ± 0.02 | 0.076 ± 0.026 | 0.032 ± 0.014 | 0.030 ± 0.002 | 0.209 ± 0.131 |
| Intestine | 0.19 ± 0.08 | 0.21 ± 0.11 | 0.132 ± 0.114 | 0.059 ± 0.036 | 0.040 ± 0.028 | 0.162 ± 0.087 |
| PIP PC3 tumor | 15.27 ± 2.93 | 22.38 ± 3.50 | 11.29 ± 2.62 | 13.98 ± 6.41 | 6.90 ± 1.87 | 4.21 ± 1.03 |
| PC3 tumor | 1.13 ± 0.61 | 0.28 ± 0.08 | 0.071 ± 0.010 | 0.080 ± 0.034 | 0.047 ± 0.012 | 0.022 ± 0.002 |

TABLE 3c
Biodistribution of [¹⁷⁷Lu]7 in tumor bearing nude mice
CD-1 male nude mice bearing PC3-PIP (PSMA positive)
and PC-3 tumors (PSMA negative)
[¹⁷⁷Lu]7 (% dose/g (Avg ± SD of n = 4)

| % dose/g | | | 24 hrs | 48 hrs | 96 hrs | 192 hrs |
|---|---|---|---|---|---|---|
| (n = 4) | 1 hr | 4 hrs | (1 day) | (2 days) | (4 days) | (8 days) |
| Blood | 13.20 ± 3.17 | 12.06 ± 2.26 | 1.79 ± 0.98 | 0.84 ± 0.25 | 0.11 ± 0.10 | 0.01 ± 0.00 |
| Heart | 3.79 ± 0.67 | 3.96 ± 0.63 | 0.73 ± 0.42 | 0.59 ± 0.21 | 0.30 ± 0.23 | 0.07 ± 0.01 |
| Muscle | 1.88 ± 0.27 | 1.55 ± 0.84 | 0.36 ± 0.24 | 0.22 ± 0.07 | 0.18 ± 0.12 | 0.03 ± 0.00 |
| Lung | 5.89 ± 0.53 | 6.07 ± 0.66 | 1.33 ± 0.85 | 0.93 ± 0.26 | 0.31 ± 0.22 | 0.09 ± 0.02 |
| Kidney | 37.38 ± 4.08 | 60.07 ± 2.41 | 18.51 ± 7.79 | 16.13 ± 6.19 | 4.28 ± 2.61 | 1.58 ± 0.43 |
| Spleen | 2.87 ± 0.82 | 6.16 ± 2.81 | 0.75 ± 0.17 | 1.27 ± 1.12 | 0.52 ± 0.30 | 0.30 ± 0.07 |
| Pancreas | 1.71 ± 0.40 | 2.08 ± 0.82 | 0.45 ± 0.30 | 0.39 ± 0.18 | 0.09 ± 0.06 | 0.03 ± 0.00 |
| Liver | 2.63 ± 0.69 | 2.92 ± 0.85 | 0.57 ± 0.27 | 0.42 ± 0.08 | 0.22 ± 0.09 | 0.12 ± 0.02 |
| Skin | 5.18 ± 1.59 | 5.85 ± 2.04 | 1.37 ± 0.63 | 0.63 ± 0.03 | 0.46 ± 0.22 | 0.16 ± 0.03 |
| Brain | 0.35 ± 0.04 | 0.37 ± 0.08 | 0.08 ± 0.04 | 0.06 ± 0.01 | 0.02 ± 0.01 | 0.01 ± 0.00 |
| Bone | 1.78 ± 0.97 | 2.05 ± 0.89 | 0.33 ± 0.14 | 0.19 ± 0.03 | 0.11 ± 0.01 | 0.09 ± 0.01 |
| Stomach | 1.57 ± 0.39 | 1.86 ± 1.12 | 0.36 ± 0.23 | 0.18 ± 0.02 | 0.09 ± 0.06 | 0.10 ± 0.09 |
| Intestine | 1.89 ± 0.18 | 2.97 ± 1.71 | 0.41 ± 0.24 | 0.23 ± 0.07 | 0.19 ± 0.18 | 0.12 ± 0.05 |
| PIP PC3 tumor | 12.51 ± 0.94 | 35.34 ± 12.11 | 34.36 ± 6.87 | 41.80 ± 10.71 | 24.23 ± 13.09 | 13.14 ± 2.57 |
| PC3 tumor | 5.26 ± 1.63 | 5.17 ± 1.02 | 2.15 ± 0.69 | 1.77 ± 0.55 | 0.97 ± 0.56 | 0.35 ± 0.03 |

Biodistribution of [⁶⁸Ga]4, [¹⁷⁷Lu]4 and [¹⁷⁷Lu]7 was determined in nude mice bearing PIP PC3 (PSMA positive) and PC3 (PSMA negative) tumors on the left and right shoulder, respectively, over a period of 192 h (Table 3a, 3b and 3c). Uptake of these radioligands into the PC-3 PIP tumors showed very different kinetic profiles. [⁶⁸Ga]4 showed excellent tumor uptake suitable for PET imaging. [¹⁷⁷Lu]4 showed a fast tumor accumulation which reached 22.38±3.50% dose/g at 4 h p.i. In the cases of [¹⁷⁷Lu]7, such high tumor uptake (35.34±12.11% dose/g) was found at 24 h and reached the highest uptake at 48hr and was retained the high level of radioactivity in PIP PC3 tumors. The uptake in PC3 tumors (PSMA negative) of both ligands, [¹⁷⁷Lu]4 and [¹⁷⁷Lu]7, was clearly much lower than those of PC-3 PIP tumors (PSMA positive). [¹⁷⁷Lu]4 showed fast clearance of radioactivity from the blood resulting in 0.02% dose/g after 4 h p.i. whereas clearance of [¹⁷⁷Lu]7 was slower resulting in 12.06% dose/g at the same time point. By introducing a 4-(p -iodophenyl) moiety as an albumin binder into, the enhanced blood circulation of [¹⁷⁷Lu]7 resulted in unprecedentedly high tumor uptake and retention over time. Results suggested that [¹⁷⁷Lu]4 and [¹⁷⁷Lu]7, might be useful for radionuclide therapy of prostate tumor over expressing PSMA binding sites.

### EXAMPLE 9

### Biodistribution of ¹⁷⁷Lu Labeled Compounds 42 and 51 in Tumor-Bearing Nude Mice

¹⁷⁷Lu labeling: To 10 μg ligand (42 or 51 in 1 mg/mL DMSO) were added 15 μL of 2.0 N NaOAc, 400 μL 0.05 N HCl, and 20 μL ¹⁷⁷Lu-solution (780 μCi (Capintec setting 450 (reading×10)). The reaction was heated with a heating block at 95° C. for 1 hour in a 3 mL closed vial. After cooling, the sample was analyzed by HPLC (HPLC: Eclipse XDB C18 150×4.6 mm, gradient, 2 mL/min; A: 0.1% TFA in water; B: 0.1% TFA in ACN: 0-2 min 100% A; 2-4 min: from 0% to 100% B; 4-9 min: 100% B; 9-10 min: from 100% to 0% B) It was found that the radiochemical purity of [¹⁷⁷Lu]42 or [¹⁷⁷Lu]51 was >98% and injected doses were stable at 24 hr after formulation. For iv injection 150 μL of labeled solution was diluted with saline to 3.75mL. Mice were injected with 150 μL of formulated dose. Injected radioactivity was 100 μCi and PSMA ligand amount was constant at 0.72 nmole/mouse.

TABLE 4a
Biodistribution of [¹⁷⁷Lu]42 in tumor bearing nude mice
CD-1 male nude mice bearing PC3-PIP (PSMA
positive) and PC-3 tumors (PSMA negative)
[¹⁷⁷Lu]42 (% dose/g, Avg ± SD of n = 4)

| | 1 hr | 4 hr | 24 hr |
|---|---|---|---|
| Blood | 0.18 ± 0.05 | 0.02 ± 0.01 | 0.00 ± 0.00 |
| Heart | 0.13 ± 0.03 | 0.06 ± 0.02 | 0.03 ± 0.01 |
| Muscle | 0.08 ± 0.02 | 0.05 ± 0.04 | 0.03 ± 0.02 |
| Lung | 0.38 ± 0.11 | 0.13 ± 0.04 | 0.04 ± 0.01 |
| Kidney | 27.52 ± 7.47 | 11.81 ± 5.81 | 2.62 ± 0.19 |
| Spleen | 0.53 ± 0.17 | 0.18 ± 0.06 | 0.04 ± 0.01 |
| Pancreas | 0.20 ± 0.09 | 0.05 ± 0.02 | 0.01 ± 0.00 |
| Liver | 0.12 ± 0.02 | 0.08 ± 0.02 | 0.06 ± 0.00 |
| Skin | 0.22 ± 0.05 | 0.10 ± 0.03 | 0.05 ± 0.01 |
| Stomach | 0.17 ± 0.07 | 0.49 ± 0.60 | 0.14 ± 0.10 |
| Lg Intestine | 0.11 ± 0.03 | 1.03 ± 1.01 | 0.37 ± 0.20 |
| Sm Intestine | 0.24 ± 0.15 | 0.94 ± 1.12 | 0.06 ± 0.05 |
| Bone | 5.82 ± 1.59 | 5.62 ± 1.18 | 4.55 ± 0.26 |
| PIP PC3 tumor | 11.29 ± 2.44 | 7.62 ± 1.60 | 4.72 ± 1.68 |
| PC3 tumor | 0.41 ± 0.10 | 0.28 ± 0.11 | 0.11 ± 0.04 |

TABLE 4b
Biodistribution of [¹⁷⁷Lu]51 in tumor bearing nude mice
CD-1 male nude mice bearing PC3-PIP (PSMA positive)
and PC-3 tumors (PSMA negative)
[¹⁷⁷Lu]51 (% dose/g, Avg ± SD of n = 4)

| | 1 hr | 4 hr | 24 hr |
|---|---|---|---|
| Blood | 0.18 ± 0.04 | 0.03 ± 0.01 | 0.00 ± 0.00 |
| Heart | 0.20 ± 0.06 | 0.05 ± 0.00 | 0.03 ± 0.01 |
| Muscle | 0.11 ± 0.03 | 0.03 ± 0.00 | 0.02 ± 0.00 |
| Lung | 0.62 ± 0.09 | 0.20 ± 0.06 | 0.10 ± 0.04 |
| Kidney | 90.59 ± 18.47 | 35.24 ± 5.28 | 4.18 ± 1.74 |
| Spleen | 1.33 ± 0.41 | 0.39 ± 0.12 | 0.15 ± 0.04 |
| Pancreas | 0.37 ± 0.11 | 0.11 ± 0.03 | 0.03 ± 0.00 |
| Liver | 0.23 ± 0.03 | 0.16 ± 0.00 | 0.14 ± 0.02 |
| Skin | 0.35 ± 0.05 | 0.11 ± 0.01 | 0.07 ± 0.02 |
| Stomach | 0.11 ± 0.03 | 0.26 ± 0.23 | 0.40 ± 0.56 |
| Lg Intestine | 0.10 ± 0.04 | 0.22 ± 0.15 | 0.49 ± 0.34 |
| Sm Intestine | 0.16 ± 0.03 | 0.21 ± 0.29 | 0.30 ± 0.33 |
| Bone | 6.52 ± 0.34 | 6.74 ± 0.71 | 5.51 ± 0.66 |
| PIP PC3 | 14.70 ± 1.29 | 15.12 ± 1.36 | 10.75 ± 2.52 |
| tumor | | | |
| PC3 tumor | 0.53 ± 0.12 | 0.20 ± 0.03 | 0.16 ± 0.02 |

Similarly, the tissue distribution of [¹⁷⁷Lu]42 and [¹⁷⁷Lu]51 was evaluated in mice bearing PIP PC3 (PSMA positive) and PC3 (PSMA negative) tumors on the left and right shoulder, respectively, over a period of 24 h (Table 4). Biodistribution data suggested that both agents showed excellent PIP PC3 (PSMA positive) tumor uptake; while the PC3 (PSMA negative) tumor as expected showed very low uptake. The tumor specific uptake for [¹⁷⁷Lu]51 was higher than that observed for [¹⁷⁷Lu]42. This finding was novel and un-expected, which suggested that the position of bisphosphonate group might have a significant impact on the in vivo biodistribution. It was found that both agents contained bisphosphonate group, which led to high specific uptake in bone. The bone uptake was consistently higher for [¹⁷⁷Lu]51. The tissue distribution of [¹⁷⁷Lu]42 and [¹⁷⁷Lu]51 suggested that these two agents might be targeting both PSMA positive tumor and perhaps might localize at lesions related to metastatic tumor in bone. Results of this study lend support for using these dual-targeted ¹⁷⁷Lu labeled agents for treatment of metastatic prostate cancer.

### EXAMPLE 10

### Biodistribution of ¹²⁵I Labeled Compounds 17, 18, 26 and 27 in Tumor-Bearing Nude Mice

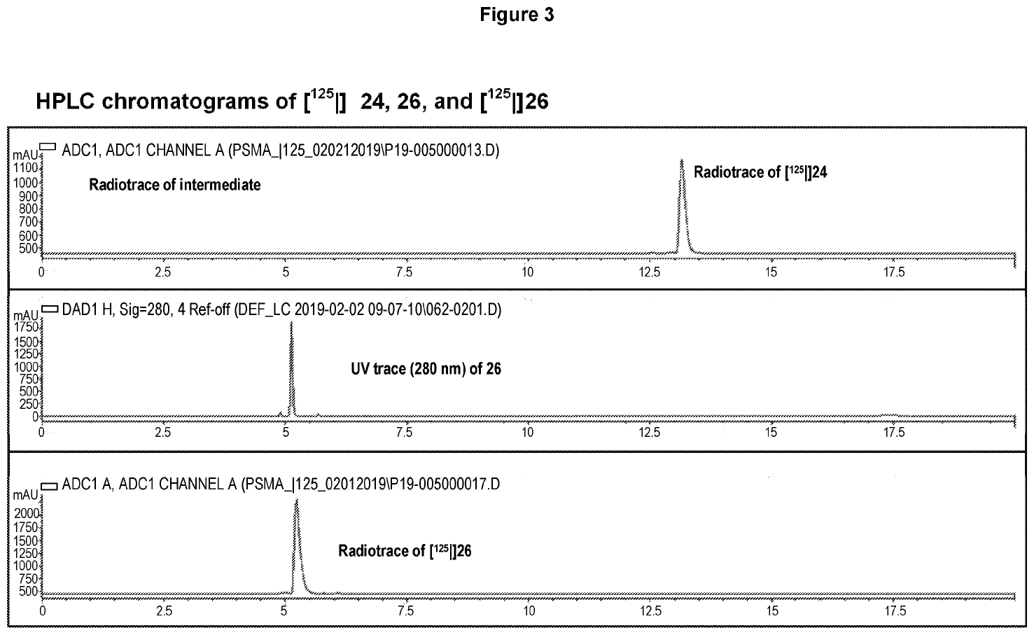

Radioiodination and Purification: 100 μg of either precursor 13, 14, 22 or 23 were dissolved in 100 μL EtOH; 22 μL Na¹²⁵I (1033-1118 μCi in 0.1 N NaOH), 100 μL 1N HCl and 100 μL 3% H₂O₂ were added. After 15 minutes at room temperature, the reaction was stopped by adding 150 μL sat. NaHSO₃. The reaction mixture was slowly added to 1.5 mL sat. NaHCO₃. Vial was rinsed with 1000 μL EtOH and mixture was diluted further to 10 mL mL water. The active sample was transferred onto an activated C4 mini column. The mixture was pushed through, washed twice with 3 mL water and the product was eluted with 1 mL ACN. Added 100 μL DMSO. The mixture was concentrated to ˜100 μL and purified by HPLC (Agilent Eclipse XCD C18 150×4.6 mm, 5 μm; 4 mL/min, gradient (ACN and water; 0-1 min (20/80), 1-16 min (20/80-100/0), 16-16.5 min (100/0-20/80), 16.5-20 min 20/80) (collected every minute). The sample was blown to dryness under argon, re-dissolved in 500 μL CH₂Cl₂ and 1 mL TFA was added at room temperature. After 1 hr the solution was blown to dryness and activity taken into 1 mL EtOH (10 μL saturated ascorbic acid/EtOH was added). Isolated activities for [¹²⁵I]17, 18, 26, and 27 were 197, 189, 600 and 197 μCi respectively. A representative picture of HPLC profiles (FIG. 3) for radiolabeled protected (intermediate), cold standard and radioactive trace of final compound is shown for [¹²⁵I]26.

For iv injection 150 μL of labeled solution was diluted with saline to 3.75 mL. Mice were injected with 2˜3 μCi of [¹²⁵I]18, 27, 26, and 18 in 0.15 mL saline. Injected radioactivity was 2 to 3 μCi.

TABLE 5
Biodistribution of [¹²⁵I]18 in tumor bearing nude mice
(% ID/g Avg. n = 4)

| % ID/g | 1 h | 4 h |
|---|---|---|
| blood | 0.71 ± 0.31 | 0.33 ± 0.17 |
| heart | 3.06 ± 0.48 | 1.60 ± 0.46 |
| muscle | 0.95 ± 0.27 | 0.69 ± 0.27 |
| lung | 3.16 ± 0.55 | 1.50 ± 0.65 |
| kidney | 96.21 ± 21.68 | 69.39 ± 24.24 |
| spleen | 6.83 ± 0.46 | 4.37 ± 1.70 |
| pancreas | 1.66 ± 0.93 | 0.89 ± 0.13 |
| liver | 3.57 ± 0.32 | 2.62 ± 1.62 |
| skin | 1.48 ± 0.44 | 0.81 ± 0.37 |
| Stomach* | 0.46 ± 0.29 | 0.72 ± 0.76 |
| large intestine* | 1.12 ± 0.65 | 0.74 ± 0.25 |
| small intestine* | 1.98 ± 0.27 | 1.11 ± 0.82 |
| gland | 4.03 ± 0.46 | 2.25 ± 0.80 |
| thyroid | 15.99 ± 1.88 | 26.99 ± 18.70 |
| Bladder* | 97.62 ± 20.87 | 123.13 ± 40.10 |
| bone | 0.89 ± 0.08 | 0.45 ± 0.23 |
| PIP tumor | 13.29 ± 3.46 | 15.27 ± 5.40 |
| PC3 tumor | 2.22 ± 0.58 | 1.20 ± 0.52 |
| PIP/blood | 21.30 ± 8.28 | 50.29 ± 15.07 |
| PIP/muscle | 16.14 ± 10.36 | 25.78 ± 14.13 |

*organ with content

TABLE 6
Biodistribution of [¹²⁵I]27 in tumor bearing nude mice
(% ID/g Avg. n = 4)

| % ID/g | 1 h | 4 h |
|---|---|---|
| blood | 0.47 ± 0.07 | 0.32 ± 0.12 |
| heart | 2.60 ± 0.10 | 1.36 ± 0.33 |
| muscle | 1.05 ± 0.07 | 0.69 ± 0.21 |
| lung | 3.53 ± 1.35 | 1.86 ± 0.24 |
| kidney | 127.77 ± 8.24 | 138.08 ± 31.03 |
| spleen | 12.25 ± 3.36 | 13.16 ± 4.01 |
| pancreas | 1.32 ± 0.46 | 1.07 ± 0.31 |
| liver | 1.72 ± 0.11 | 1.28 ± 0.63 |
| skin | 2.68 ± 0.47 | 2.03 ± 0.39 |
| Stomach* | 0.55 ± 0.21 | 0.96 ± 0.55 |
| large intestine* | 1.13 ± 0.52 | 1.35 ± 0.48 |
| small intestine* | 1.52 ± 0.45 | 0.83 ± 0.34 |
| gland | 3.59 ± 1.03 | 3.57 ± 0.54 |
| thyroid | 39.72 ± 12.95 | 60.34 ± 22.28 |
| Bladder* | 53.39 ± 19.82 | 38.17 ± 21.99 |
| bone | 0.94 ± 0.21 | 0.56 ± 0.05 |
| PIP tumor | 17.19 ± 3.74 | 17.36 ± 2.57 |
| PC3 tumor | 1.99 ± 0.17 | 1.67 ± 0.44 |
| PIP/blood | 36.90 ± 8.14 | 58.93 ± 20.54 |
| PIP/muscle | 16.57 ± 4.28 | 27.34 ± 10.28 |

*organ with content

TABLE 7
Biodistribution of [¹²⁵I]17 in tumor bearing nude mice
(% ID/g Avg. n = 4)

| % ID/g | 1 h | 4 h |
|---|---|---|
| blood | 0.45 ± 0.11 | 0.09 ± 0.03 |
| heart | 0.29 ± 0.04 | 0.11 ± 0.07 |
| muscle | 0.43 ± 0.24 | 0.25 ± 0.44 |
| lung | 1.15 ± 1.27 | 0.19 ± 0.13 |
| kidney | 41.44 ± 5.10 | 25.41 ± 7.57 |
| spleen | 3.25 ± 1.52 | 0.46 ± 0.27 |
| pancreas | 0.55 ± 0.64 | 0.22 ± 0.25 |
| liver | 2.94 ± 2.12 | 0.48 ± 0.53 |
| skin | 0.57 ± 0.24 | 0.11 ± 0.04 |
| Stomach* | 0.18 ± 0.05 | 0.11 ± 0.10 |
| large intestine* | 2.10 ± 1.64 | 0.86 ± 0.22 |
| small intestine* | 3.22 ± 1.64 | 0.23 ± 0.16 |
| gland | 0.81 ± 0.18 | 0.19 ± 0.05 |
| thyroid | 3.57 ± 0.75 | 7.16 ± 1.91 |
| Bladder* | 204.72 ± 108.11 | 68.75 ± 30.09 |
| bone | 0.16 ± 0.02 | 0.03 ± 0.02 |
| PIP tumor | 10.72 ± 2.98 | 5.17 ± 2.02 |
| PC3 tumor | 1.70 ± 1.33 | 0.28 ± 0.10 |
| PIP/blood | 23.69 ± 2.07 | 62.95 ± 31.81 |
| PIP/muscle | 27.52 ± 7.80 | 200.29 ± 221.74 |

*organ with content

TABLE 8
Biodistribution of [¹²⁵I]126 in tumor bearing nude mice
(% ID/g Avg. n = 4)

| % ID/g | 1 h | 4 h |
|---|---|---|
| blood | 0.46 ± 0.22 | 0.21 ± 0.21 |
| heart | 0.73 ± 0.20 | 0.38 ± 0.23 |
| muscle | 1.72 ± 0.54 | 0.30 ± 0.19 |
| lung | 3.08 ± 0.87 | 2.08 ± 0.67 |
| kidney | 174.61 ± 30.65 | 114.69 ± 32.67 |
| spleen | 21.66 ± 2.63 | 15.37 ± 3.26 |
| pancreas | 0.98 ± 0.42 | 0.52 ± 0.26 |
| liver | 4.61 ± 1.72 | 4.90 ± 1.80 |
| skin | 2.33 ± 0.61 | 1.67 ± 1.30 |
| Stomach* | 0.35 ± 0.11 | 0.49 ± 0.27 |
| large intestine* | 1.24 ± 0.79 | 1.35 ± 0.47 |
| small intestine* | 1.08 ± 0.63 | 0.30 ± 0.11 |
| gland | 6.97 ± 1.06 | 2.03 ± 1.29 |
| thyroid | 13.21 ± 8.48 | 28.57 ± 10.04 |
| Bladder* | 14.69 ± 3.80 | 15.11 ± 4.09 |
| bone | 0.48 ± 0.11 | 0.25 ± 0.06 |
| PIP tumor | 26.19 ± 3.46 | 14.32 ± 3.87 |
| PC3 tumor | 2.39 ± 1.42 | 1.37 ± 0.45 |
| PIP/blood | 66.04 ± 29.31 | 104.80 ± 62.05 |
| PIP/muscle | 16.02 ± 3.85 | 60.91 ± 38.35 |

*organ with content

Biodistribution study of [¹²⁵I]18, 27, 26, and 18 in tumor bearing nude mice were evaluated for their ability to localization of PSMA positive tumor (Tables 5, 6, 7 and 8). It was observed that [¹²⁵I]26 and [¹²⁵I] 27 containing three benzene rings in the molecule showed higher uptakes in PIP tumors, kidneys and spleen compared to [¹²⁵I]17 and [¹²⁵I18. Results suggested that compounds with higher lipophilicity showed stronger binding affinity to PSMA in vivo. [¹²⁵I]17 and [¹²⁵I]26 with one additional glutamic acid in the linker showed significantly faster washout in PIP tumors, kidneys and spleen compared to [¹²⁵I]18 and [¹²⁵I]27. The observation indicated that the lipophilicity and in vivo biodistribution may be manipulated by adding lipophilic benzene rings or hydrophilic glutamic acid in the linker. Liver uptakes were low, indicating that [¹²⁵I]18, 27, 26, and 18 -PSMA compounds were preferentially excreted via the renal system rather than the hepatobiliary route. These new agents are valuable for radionuclide therapy, when labeled with beta or alpha-emitting isotopes; but these agents will also be useful as diagnostic agents when labeled with gamma-emitting isotopes.

While certain embodiments have been illustrated and described, it should be understood that changes and modifications can be made therein in accordance with ordinary skill in the art without departing from the technology in its broader aspects as defined in the following claims.

The present disclosure is not to be limited in terms of the particular embodiments described in this application. Modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### Abbreviations:

SPECT, single photon emission computer tomography;
PET, positron emission tomography
HPLC, High performance liquid chromatography;
HRMS, High-resolution mass spectrometry;
PBS, phosphate buffered saline;
SPE, solid-phase extraction;
TFA, trifluoroacetic acid;
GMP: manufacturing good manufacturing;
NET: neuroendocrine tumor
FDG, 2-fluoro-2-dexoy-D-glucose
DOTA: 1,4,7,10-tetraazacyclododecane-1,4,7,10-tetraacetic acid
DOTA-TOC, DOTA-D-Phe-c(Cys-Tyr-D-Trp-Lys-Thr-Cys)-Thr-ol
DOTA-TATE, DOTA-D-Phe-c(Cys-Tyr-D-Trp-Lys-Thr-Cys)-Thr
DOTA-NOC, DOTA-D-Phe-c(Cys-Nal-D-Trp-Lys-Thr-Cys)-Thr-ol
NOTA: 1,4,7-triazacyclononane-N,N′,N″-triacetic acid
NODAGA: 1,4,7-triazacyclononane, 1-glutaric acid-4,7-acetic acid
DOTAGA: 1,4,7,10-tetraazacyclodocecane,1-(glutaric acid)-4,7,10-triacetic acid
DOTA(GA)2: 1,4,7,10-tetraazacyclodocecane,1,7-(diglutaric acid)-4,10-diacetic acid
TRAP: 1,4,7-triazacyclononane-N,N′,N″-tris(methylenephosphonic) acid
DEDPA: 1,2-[[6-(carboxy)-pyridin-2-yl]-methylamino]ethane
AAZTA: 6-[bis(hydroxycarbonyl-methyl)amino]-1,4-bis(hydroxycarbonyl methyl)-6-methylperhydro-1,4-diazepine,
EDTMP (ethylene-diamino-N,N,N′,N′-tetrakis-methylene-phosphoric acid) bis-(Glu-NH—CO—NH-Lys-(Ahx)-HBED-CC)
[¹¹C]-MCG: [¹¹C](S)-2-[3-((R)-1-carboxy-2-methylsulfanyl-ethyl)-ureido]-pentanedioic acid,
[¹⁸F]DCFBC: N-[N-[(S)-1,3-dicarboxypropyl]carbamoyl]-4-[¹⁸F]-fluorobenzyl-L-cysteine,
[¹⁸F]DCFPyL: 2-(3-(1-carboxy-5-[(6-[¹⁸]fluoro-pyridine-3-carbonyl)-amino]-pentyl)-ureido)-pentanedioic acid,
PSMA-11 Glu-NH—CO—NH-Lys-(Ahx)-(HBED-CC)
PSMA-617: 2-[3-(1-Carboxy-5-(3-naphthalen-2-yl-2-[(4-([2-(4,7,10-tris-carboxy methyl-1,4,7,10-tetraaza-cyclododec-1-yl)-acetylamino]-methyl)-cyclohexanecarbonyl)-amino]-Propionylamino)-pentyl)-ureido]-pentanedioic acid
GPI 2[(3-amino-3-carboxypropyl)(hydroxy)(phosphinyl)-methyl]pentane-1,5-dioic acid
2-PMPA 2-(3-mercaptopropyl)pentane-dioic acid

### REFERENCES

[1] Afshar-Oromieh A, Haberkorn U, Zechmann C, Armor T, Mier W, Spohn F, et al. Repeated PSMA-targeting radioligand therapy of metastatic prostate cancer with (131)I-MIP-1095. Eur. J. Nucl. Med. Mol. Imaging 2017;44:950-9.
[2] Reyes D K, Demehri S, Werner R A, Pomper M G, Gorin M A, Rowe S P, et al. PSMA-targeted [(18)F]DCFPyL PET/CT-avid lesions in a patient with prostate cancer: Clinical decision-making informed by the PSMA-RADS interpretive framework. Urol Case Rep 2019;23 :72-4.
[3] Giesel F L, Knorr K, Spohn F, Will L, Maurer T, Flechsig P, et al. Detection efficacy of [(18)F]PSMA-1007 PET/CT in 251 Patients with biochemical recurrence after radical prostatectomy. J. Nucl. Med. 2018.
[4] Fendler W P, Calais J, Eiber M, Flavell R R, Mishoe A, Feng F Y, et al. Assessment of 68Ga-PSMA-11 PET Accuracy in Localizing Recurrent Prostate Cancer: A Prospective Single-Arm Clinical Trial. JAMA Oncol 2019.
[5] Zha Z, Ploessl K, Choi S R, Wu Z, Zhu L, and Kung H F. Synthesis and evaluation of a novel urea-based (68)Ga-complex for imaging PSMA binding in tumor. Nucl. Med. Biol. 2018;59:36-47.
[6] Velikyan I. 68Ga-Based Radiopharmaceuticals: Production and Application Relationship. Molecules 2015;20:12913-43.
[7] Banerjee S, Pillai M R, and Knapp F F. Lutetium-177 therapeutic radiopharmaceuticals: linking chemistry, radiochemistry, and practical applications. Chem. Rev. 2015;115:2934-74.
[8] Kostelnik T I and Orvig C. Radioactive Main Group and Rare Earth Metals for Imaging and Therapy. Chem. Rev. 2018.
[9] Ballinger JR. Theranostic radiopharmaceuticals: established agents in current use. Br. J. Radiol. 2018;91:20170969.
[10] Kratochwil C, Haberkorn U, and Giesel F L. Radionuclide Therapy of Metastatic Prostate Cancer. Semin. Nucl. Med.: Elsevier; 2019.
[11] Hofman M S, Hicks R J, Maurer T, and Eiber M. Prostate-specific Membrane Antigen PET: Clinical Utility in Prostate Cancer, Normal Patterns, Pearls, and Pitfalls. Radiographics 2018;38:200-17.
[12] O'Keefe D S, Bacich D J, Huang S S, and Heston WDW. A Perspective on the Evolving Story of PSMA Biology, PSMA-Based Imaging, and Endoradiotherapeutic Strategies. J. Nucl. Med. 2018;59:1007-13.
[13] Rowe S P, Gorin M A, and Pomper M G. Imaging of Prostate-Specific Membrane Antigen with Small-Molecule PET Radiotracers: From the Bench to Advanced Clinical Applications. Annu. Rev. Med. 2019;70:461-77.
[14] Wustemann T, Haberkorn U, Babich J, and Mier W. Targeting prostate cancer: Prostate-specific membrane antigen based diagnosis and therapy. Med. Res. Rev. 2019;39:40-69.
[15] Kulkarni H R, Singh A, Langbein T, Schuchardt C, Mueller D, Zhang J, et al. Theranostics of prostate cancer: from molecular imaging to precision molecular radiotherapy targeting the prostate specific membrane antigen. Br. J. Radiol. 2018;91:20180308.
[16] Emmett L, Crumbaker M, Ho B, Willowson K, Eu P, Ratnayake L, et al. Results of a Prospective Phase 2 Pilot Trial of (177)Lu-PSMA-617 Therapy for Metastatic Castration-Resistant Prostate Cancer Including Imaging Predictors of Treatment Response and Patterns of Progression. Clin. Genitourin. Cancer 2019;17:15-22.
[17] Heck M M, Tauber R, Schwaiger S, Retz M, D'Alessandria C, Maurer T, et al. Treatment Outcome, Toxicity, and Predictive Factors for Radioligand Therapy with (177)Lu-PSMA-I&amp; T in Metastatic Castration-resistant Prostate Cancer Post-therapeutic dosimetry of 177Lu-DKFZ-PSMA-617 in the treatment of patients with metastatic castration-resistant prostate cancer. Eur. Urol. 2018;38:91-8.
[18] Tsionou M I, Knapp C E, Foley C A, Munteanu C R, Cakebread A, Imberti C, et al. Comparison of macrocyclic and acyclic chelators for gallium-68 radiolabelling. RSC Adv 2017;7:49586-99.
[19] Price E W and Orvig C. Matching chelators to radiometals for radiopharmaceuticals. Chem. Soc. Rev. 2014;43:260-90.
[20] Stasiuk G J and Long N J. The ubiquitous DOTA and its derivatives: the impact of 1,4,7,10-tetraazacyclododecane-1,4,7,10-tetraacetic acid on biomedical imaging. Chem. Commun. (Camb.) 2013;49:2732-46.
[21] Roosenburg S, Laverman P, Joosten L, Cooper M S, Kolenc-Peitl P K, Foster J M, et al. PET and SPECT imaging of a radiolabeled minigastrin analogue conjugated with DOTA, NOTA, and NODAGA and labeled with (64)Cu, (68)Ga, and (111)In. Mol. Pharm. 2014;11:3930-7.
[22] Notni J, Simecek J, and Wester H J. Phosphinic acid functionalized polyazacycloalkane chelators for radiodiagnostics and radiotherapeutics: unique characteristics and applications. ChemMedChem 2014;9:1107-15.
[23] Baum R P, Kulkarni H R, Muller D, Satz S, Danthi N, Kim Y S, et al. First-In-Human Study Demonstrating Tumor-Angiogenesis by PET/CT Imaging with (68)Ga-NODAGA-THERANOST, a High-Affinity Peptidomimetic for alphavbeta3 Integrin Receptor Targeting. Cancer Biother. Radiopharm. 2015;30:152-9.
[24] Eisenwiener K P, Prata M I, Buschmann I, Zhang H W, Santos A C, Wenger S, et al. NODAGATOC, a new chelator-coupled somatostatin analogue labeled with [67/68Ga] and [111In] for SPECT, PET, and targeted therapeutic applications of somatostatin receptor (hsst2) expressing tumors. Bioconjug. Chem. 2002;13:530-41.
[25] Boros E, Ferreira C L, Yapp D T, Gill R K, Price E W, Adam M J, et al. RGD conjugates of the H2dedpa scaffold: synthesis, labeling and imaging with 68Ga. Nucl. Med. Biol. 2012;39:785-94.
[26] Manzoni L, Belvisi L, Arosio D, Bartolomeo MP, Bianchi A, Brioschi C, et al. Synthesis of Gd and (68)Ga complexes in conjugation with a conformationally optimized RGD sequence as potential MRI and PET tumor-imaging probes. ChemMedChem 2012;7:1084-93.
[27] Waldron B P, Parker D, Burchardt C, Yufit DS, Zimny M, and Roesch F. Structure and stability of hexadentate complexes of ligands based on AAZTA for efficient PET labelling with gallium-68. Chem. Commun. (Camb.) 2013;49:579-81.
[28] Pomper M G, Musachio J L, Zhang J, Scheffel U, Zhou Y, Hilton J, et al. 11C-MCG: synthesis, uptake selectivity, and primate PET of a probe for glutamate carboxypeptidase II (NAALADase). Mol. Imaging 2002;1:96-101.
[29] Rowe S P, Gage K L, Faraj S F, Macura K J, Cornish T C, Gonzalez-Roibon N, et al. (1)(8)F-DCFBC PET/CT for PSMA-Based Detection and Characterization of Primary Prostate Cancer. J. Nucl. Med. 2015;56:1003-10.
[30] Cho S Y, Gage K L, Mease R C, Senthamizhchelvan S, Holt D P, Jeffrey-Kwanisai A, et al. Biodistribution, tumor detection, and radiation dosimetry of 18F-DCFBC, a low-molecular-weight inhibitor of prostate-specific membrane antigen, in patients with metastatic prostate cancer. J. Nucl. Med. 2012;53:1883-91.
[31] Chen Y, Pullambhatla M, Foss C A, Byun Y, Nimmagadda S, Senthamizhchelvan S, et al. 2-(3-{1-Carboxy-5-[(6-[18F]fluoro-pyridine-3 -carbonyl)-amino]-pentyl}-ureido)-pen tanedioic acid, [18F]DCFPyL, a PSMA-based PET imaging agent for prostate cancer. Clin. Cancer Res. 2011;17:7645-53.
[32] Szabo Z, Mena E, Rowe S P, Plyku D, Nidal R, Eisenberger M A, et al. Initial Evaluation of [(18)F]DCFPyL for Prostate-Specific Membrane Antigen (PSMA)-Targeted PET Imaging of Prostate Cancer. Mol. Imaging Biol. 2015;17:565-74.
[33] Eder M, Eisenhut M, Babich J, and Haberkorn U. PSMA as a target for radiolabelled small molecules. Eur. J. Nucl. Med. Mol. Imaging 2013;40:819-23.
[34] Eder M, Neels O, Mueller M, Bauder-Wuest U, Remde Y, Schaefer M, et al. Novel preclinical and radiopharmaceutical aspects of [68Ga]Ga-PSMA-HBED-CC: a new PET tracer for imaging of prostate cancer. Pharmaceuticals 2014;7:779-96.
[35] Eiber M, Maurer T, Souvatzoglou M, Beer A J, Ruffani A, Haller B, et al. Evaluation of Hybrid 68Ga-PSMA Ligand PET/CT in 248 Patients with Biochemical Recurrence After Radical Prostatectomy. J. Nucl. Med. 2015;56:668-74.
[36] Benesova M, Schafer M, Bauder-Wust U, Afshar-Oromieh A, Kratochwil C, Mier W, et al. Preclinical Evaluation of a Tailor-Made DOTA-Conjugated PSMA Inhibitor with Optimized Linker Moiety for Imaging and Endoradiotherapy of Prostate Cancer. J. Nucl. Med. 2015;56:914-20.
[37] Kabasakal L, AbuQbeitah M, Aygun A, Yeyin N, Ocak M, Demirci E, et al. Pre-therapeutic dosimetry of normal organs and tissues of Lu-PSMA-617 prostate-specific membrane antigen (PSMA) inhibitor in patients with castration-resistant prostate cancer. Eur. J. Nucl. Med. Mol. Imaging 2015.
[38] Afshar-Oromieh A, Hetzheim H, Kratochwil C, Benesova M, Eder M, Neels O C, et al. The novel theranostic PSMA-ligand PSMA-617 in the diagnosis of prostate cancer by PET/CT: biodistribution in humans, radiation dosimetry and first evaluation of tumor lesions. J. Nucl. Med. 2015;56:1697-705.
[39] Weineisen M, Schottelius M, Simecek J, Baum R P, Yildiz A, Beykan S, et al. 68Ga- and 177Lu-Labeled PSMA I&T: Optimization of a PSMA-Targeted Theranostic Concept and First Proof-of-Concept Human Studies. J. Nucl. Med. 2015;56:1169-76.
[40] Herrmann K, Bluemel C, Weineisen M, Schottelius M, Wester H J, Czernin J, et al. Biodistribution and radiation dosimetry for a probe targeting prostate-specific membrane antigen for imaging and therapy. J. Nucl. Med. 2015;56:855-61.

What is claimed is:

1. A compound according to Formula I: 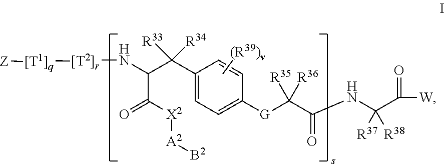
or a pharmaceutically acceptable salt thereof,
wherein
Z is a chelating moiety, or
a group having the structure of Z¹: 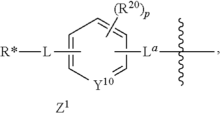
wherein Y¹⁰ is CH or N;
each of L and L^{a} is independently a bond or a divalent linking moiety comprising 1 to 6 carbon atoms in a chain, a ring, or a combination thereof, wherein at least one carbon atom is optionally replaced with O, —NR³—, or —C(O)—;
R* is a radioactive isotope;
R²² is selected from the group consisting of alkyl, alkoxyl, halide, haloalkyl, and CN;
p is an integer from 0 to 4, wherein when p is greater than 1, each R²² is the same or different;
W is a PSMA-targeting ligand;
each T¹ independently has the structure of T¹¹ or T¹²: 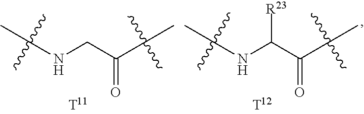
wherein R²³ is —(CH₂)ₐCO₂H, and a is an integer from 0 to 4;
each T² independently has the structure of of T²¹ or T²²: 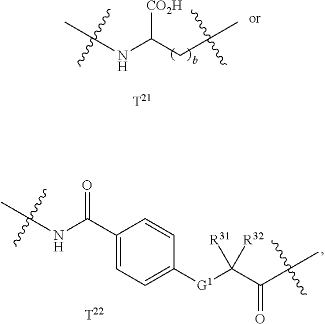
wherein b is an integer from 1 to 6, and G¹ is O, S, or NR³;
q is 0, 1, 2, or 3;
r is 0, 1, or 2;
A² is a bond or a divalent linking moiety comprising 1 to 20 carbon atoms in a chain, a ring, or a combination thereof, wherein one or more carbon atoms can be optionally replaced with O, —NR⁴⁰—, or —C(O)—;
B² is H 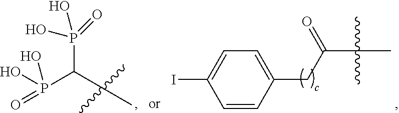
wherein c is an integer from 1 to 4,
G is O, S, or NR³;
X² is O, S, or —NR⁴¹—;
each of R³, R⁴⁰, and R⁴¹ is independently selected from the group consisting of hydrogen, alkyl, cycloalkyl, heterocycloalkyl, aryl, alkylaryl, and heteroaryl.
each of R³¹, R³², R³³, R³⁴, R³⁵, and R³⁶ is independently hydrogen, alkyl, alkoxyl, or halide;
each of R³⁷ and R³⁸ is independently hydrogen, alkyl, aryl, or alkylaryl;
each R³⁹ is independently selected from the group consisting of alkyl, alkoxyl, halide, haloalkyl, and CN;
s is 0 or 1; and
v is an integer from 0 to 4, wherein when v is greater than 1, each R³⁹ is the same or different;
provided that if s is 1, —X²-A²-B² is —OH, r is 0, q is 1, and T¹ is T¹¹, then Z is not Z¹ or

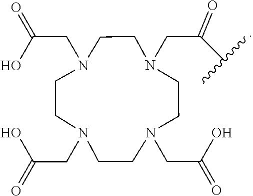 [SMILES: *C(=O)CN1CCN(CC(=O)O)CCN(CC(=O)O)CCN(CC(=O)O)CC1]

2. The compound of claim 1, or a pharmaceutically acceptable salt thereof, wherein Z is 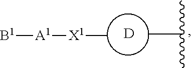
A¹ is a bond or a divalent linking moiety comprising 1 to 20 carbon atoms in a chain, a ring, or a combination thereof, wherein one or more carbon atoms can be optionally replaced with O, —NR⁴⁰—, or —C(O)—;
B¹ is H, 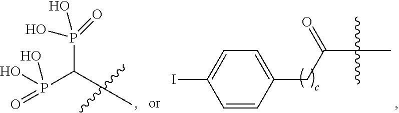
wherein c is an integer from 1 to 4;
X¹ is O, S, or —NR⁴¹—; and
D is a divalent chelating group derived from 1,4,7,10-tetraazacyclododecane-1,4,7,10-tetraacetic acid.

3. The compound of claim 2, or a pharmaceutically acceptable salt thereof, wherein D is selected from the group consisting of:

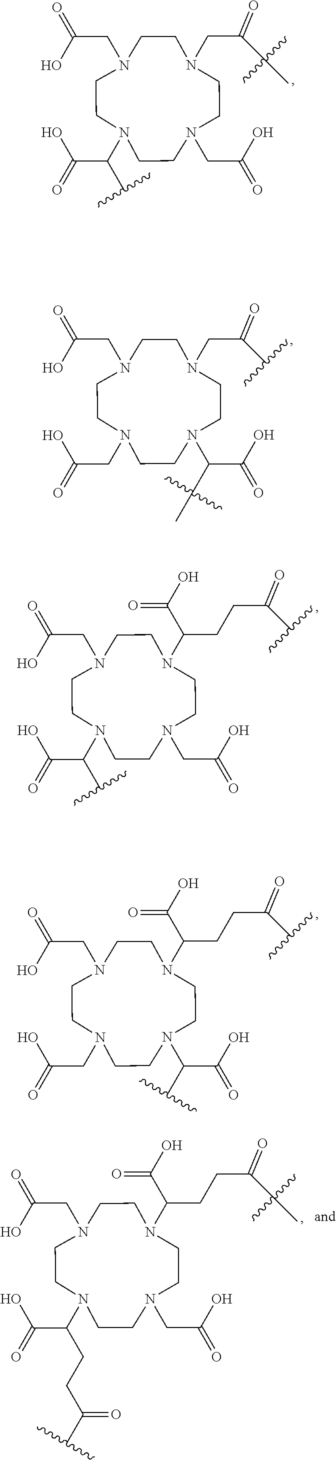 [SMILES: *C(=O)CN1CCN(CC(=O)O)CCN(C(*)C(=O)O)CCN(CC(=O)O)CC1 ; *C(=O)CN1CCN(CC(=O)O)CCN(CC(=O)O)CCN(C(*)C(=O)O)CC1 ; *C(=O)CCC(C(=O)O)N1CCN(CC(=O)O)CCN(C(*)C(=O)O)CCN(CC(=O)O)CC1 ; *C(=O)CCC(C(=O)O)N1CCN(CC(=O)O)CCN(CC(=O)O)CCN(C(*)C(=O)O)CC1 ; *C(=O)CCC(C(=O)O)N1CCN(CC(=O)O)CCN(C(CCC(*)=O)C(=O)O)CCN(CC(=O)O)CC1]

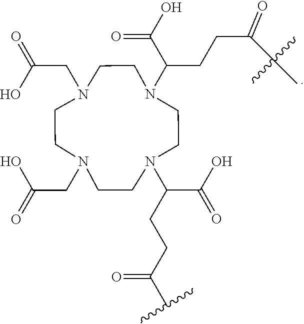 [SMILES: *C(=O)CCC(C(=O)O)N1CCN(CC(=O)O)CCN(CC(=O)O)CCN(C(CCC(*)=O)C(=O)O)CC1]

4. The compound of claim 3, or a pharmaceutically acceptable salt thereof, wherein D is selected from the group consisting of:

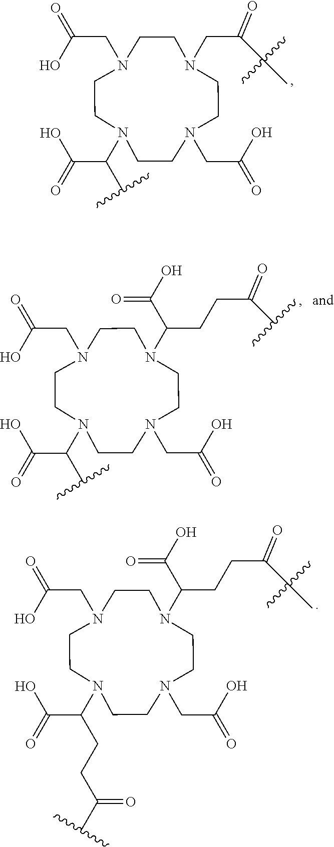 [SMILES: *C(=O)CN1CCN(CC(=O)O)CCN(C(*)C(=O)O)CCN(CC(=O)O)CC1 ; *C(=O)CCC(C(=O)O)N1CCN(CC(=O)O)CCN(C(*)C(=O)O)CCN(CC(=O)O)CC1 ; *C(=O)CCC(C(=O)O)N1CCN(CC(=O)O)CCN(C(CCC(*)=O)C(=O)O)CCN(CC(=O)O)CC1]

5. The compound of claim 1, or a pharmaceutically acceptable salt thereof, wherein Z is Z¹ having the structure: 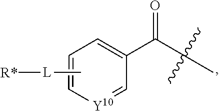
wherein R* is ¹²³I, ¹²⁴I, ¹²⁵I, ¹³¹I, ¹⁸F, or ²¹¹As.

6. The compound of claim 5, or a pharmaceutically acceptable salt thereof, wherein Z¹ has the structure:

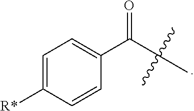

7. The compound of any one of claims 1 to 6, or a pharmaceutically acceptable salt thereof, wherein W has the structure: 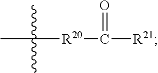
and
R²° and R²¹ are each independently an amino acid residue linked via an amino group thereof to the adjacent —C(O)— group.

8. The compound of any one of claims 1 to 6, or a pharmaceutically acceptable salt thereof, wherein W has the structure: 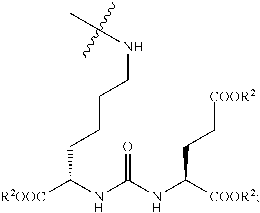
and
R² is hydrogen or a carboxylic acid protecting group.

9. The compound of any one of claims 1 to 8, having Formula I-A: 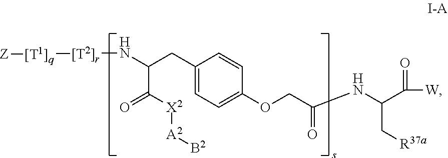
or a pharmaceutically acceptable salt thereof, wherein R^{37a} is optionally substituted phenyl or optionally substituted naphthyl.

10. The compound of claim 9, having Formula I-B: 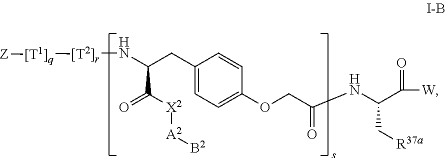
or a pharmaceutically acceptable salt thereof.

11. The compound of any one of claims 1 to 10, having Formula II-A: 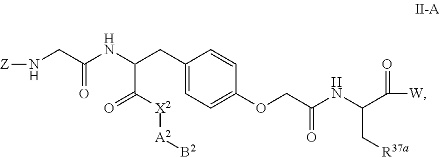
or a pharmaceutically acceptable salt thereof.

12. The compound of any one of claims 1 to 10, having Formula II-B: 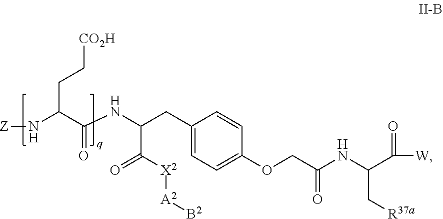
or a pharmaceutically acceptable salt thereof,
wherein q is 1 or 2.

13. The compound of any one of claims 1 to 10, having Formula II-C: 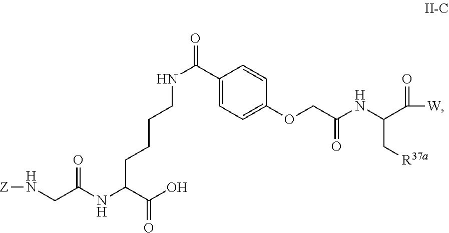
or a pharmaceutically acceptable salt thereof.

14. The compound of any one of claims 1 to 10, having Formula II-D: 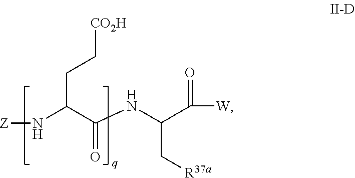
or a pharmaceutically acceptable salt thereof,
wherein q is 1 or 2.

15. The compound of any one of claims 1 to 4 and 7 to 14, or a pharmaceutically acceptable salt thereof, wherein Z is 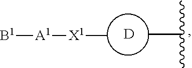
A¹ is a bond or a divalent linking moiety comprising 1 to 20 carbon atoms in a chain, a ring, or a combination thereof, wherein one or more carbon atoms can be optionally replaced with O, —NH—, or —C(O)—;
B¹ is H, 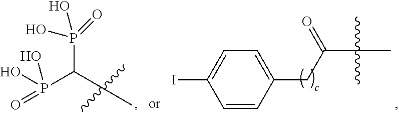
wherein c is 3;
X¹ is a bond, O, or —NH—; and
D is

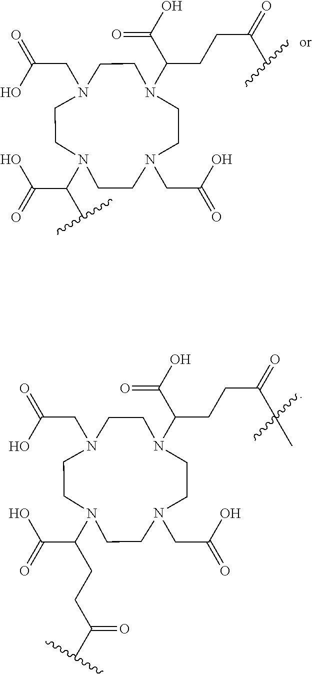 [SMILES: *C(=O)CCC(C(=O)O)N1CCN(CC(=O)O)CCN(C(*)C(=O)O)CCN(CC(=O)O)CC1 ; *C(=O)CCC(C(=O)O)N1CCN(CC(=O)O)CCN(C(CCC(*)=O)C(=O)O)CCN(CC(=O)O)CC1]

16. The compound of any one of claims 1 to 4 and 7 to 10, having Formula III-A: 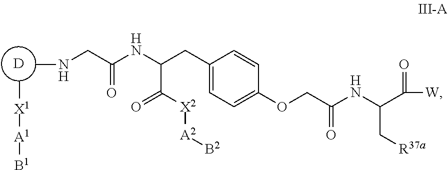
or a pharmaceutically acceptable salt thereof.

17. The compound of claim 16, having Formula III-B: 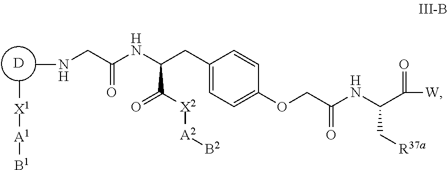
or a pharmaceutically acceptable salt thereof.

18. The compound of claim 17, having Formula IV-A: 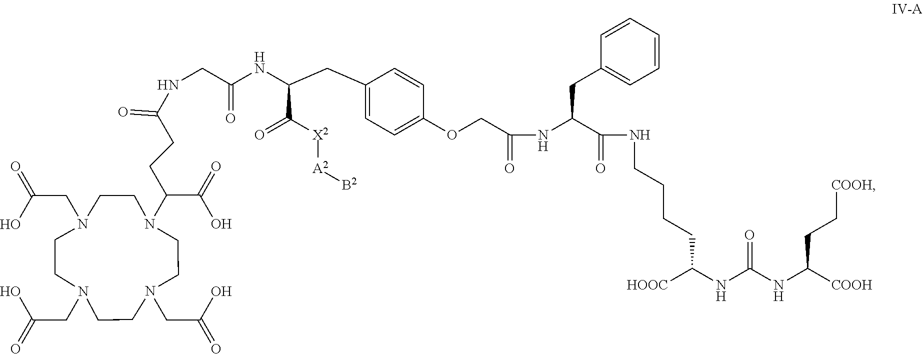
or a pharmaceutically acceptable salt thereof.

19. The compound of claim 17, having Formula IV-B: 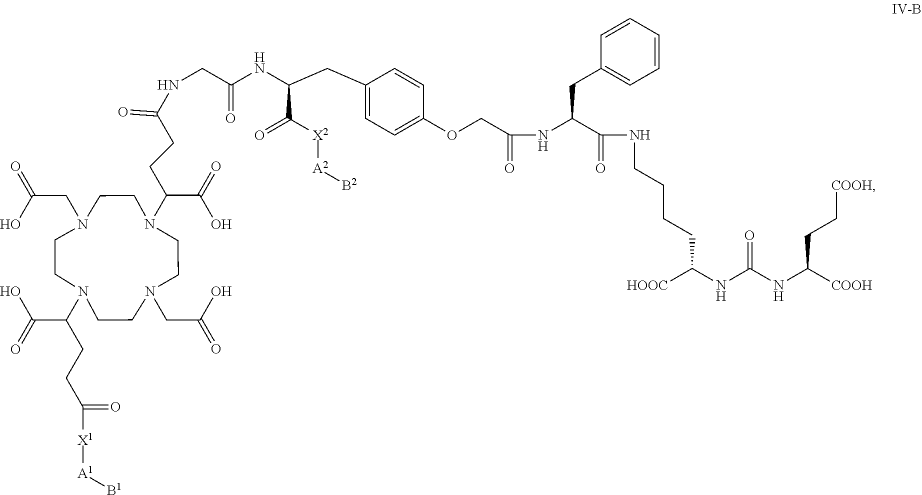
or a pharmaceutically acceptable salt thereof.

20. The compound of any one of claims 9 to 17, wherein R^{37a} is optionally substituted phenyl.

21. The compound of any one of claims 2 to 4, 7 to 17, and 19 to 20, or a pharmaceutically acceptable salt thereof, wherein X¹ is O or —NH—.

22. The compound of any one of claims 1 to 21, or a pharmaceutically acceptable salt thereof, wherein X² is a bond, O, or —NH—.

23. The compound of any one of claims 1 to 22, or a pharmaceutically acceptable salt thereof, wherein each of A¹ and A² is a bond, —(CH₂)ₙ—, —(CH₂)ₙC(O)O—, —(CH₂)ₙC(O)NH—, —(CH₂CH₂O)ₙ—, or —(CH₂CH₂O)ₙ(CH₂CH₂NH)ₙ—; and
each n is independently 1, 2, 3, or 4.

24. The compound of claim 23, or a pharmaceutically acceptable salt thereof, wherein A¹ is a bond, —(CH₂)ₙC(O)NH—, or —(CH₂CH₂O)ₙ(CH₂CH₂NH)ₙ—.

25. The compound of claim 24, or a pharmaceutically acceptable salt thereof, wherein A¹ is a bond, —(CH₂)C(O)NH—, or —(CH₂CH₂O)₂(CH₂CH₂NH)—.

26. The compound of any one of claims 1 to 25, or a pharmaceutically acceptable salt thereof, wherein A² is a bond or —(CH₂)ₙC(O)NH—; and n is 1, 2, or 3.

27. The compound of claim 26, or a pharmaceutically acceptable salt thereof, wherein A² is a bond or —(CH₂)C(O)NH—.

28. The compound of claim 1, having the structure: 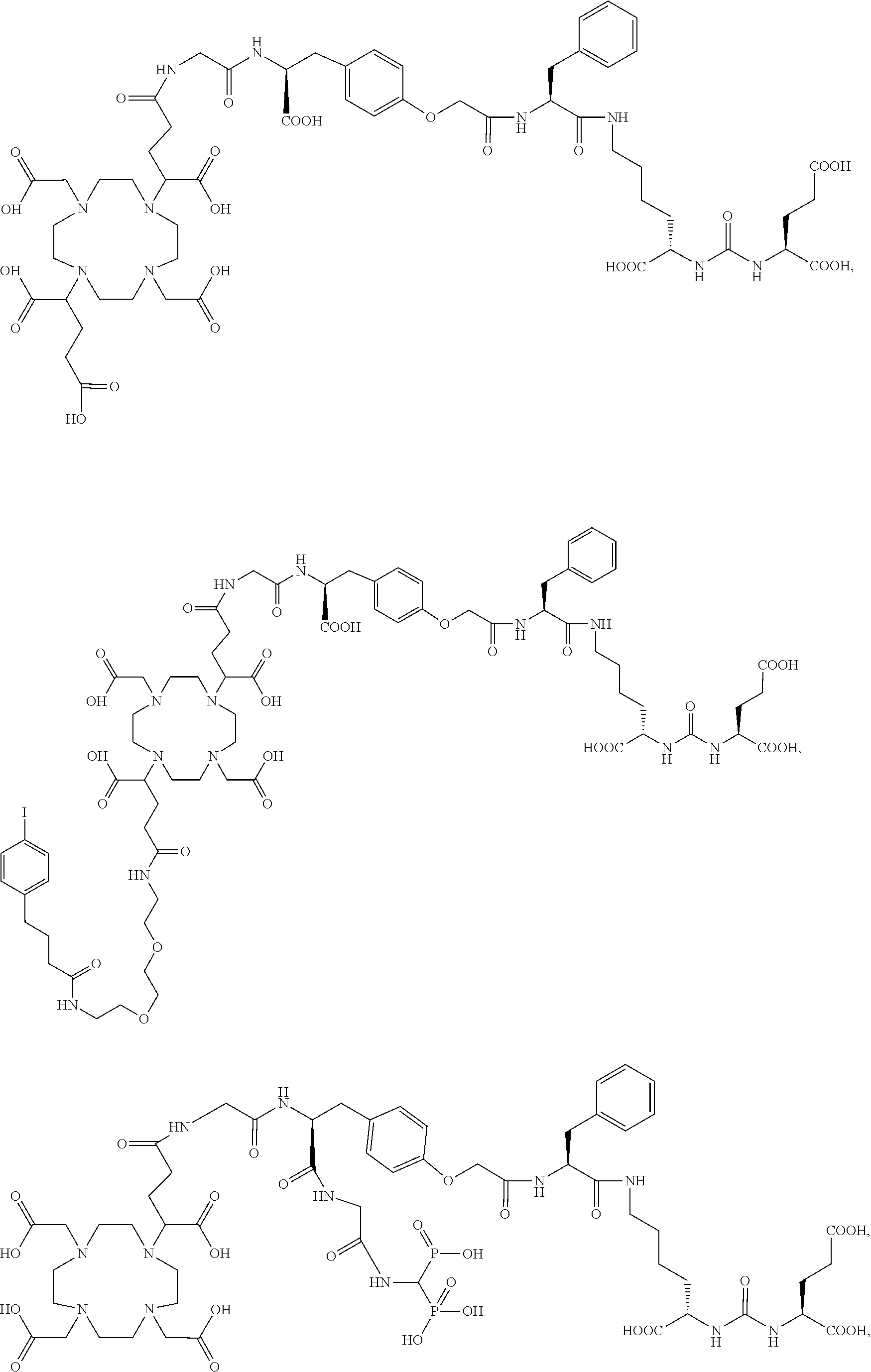 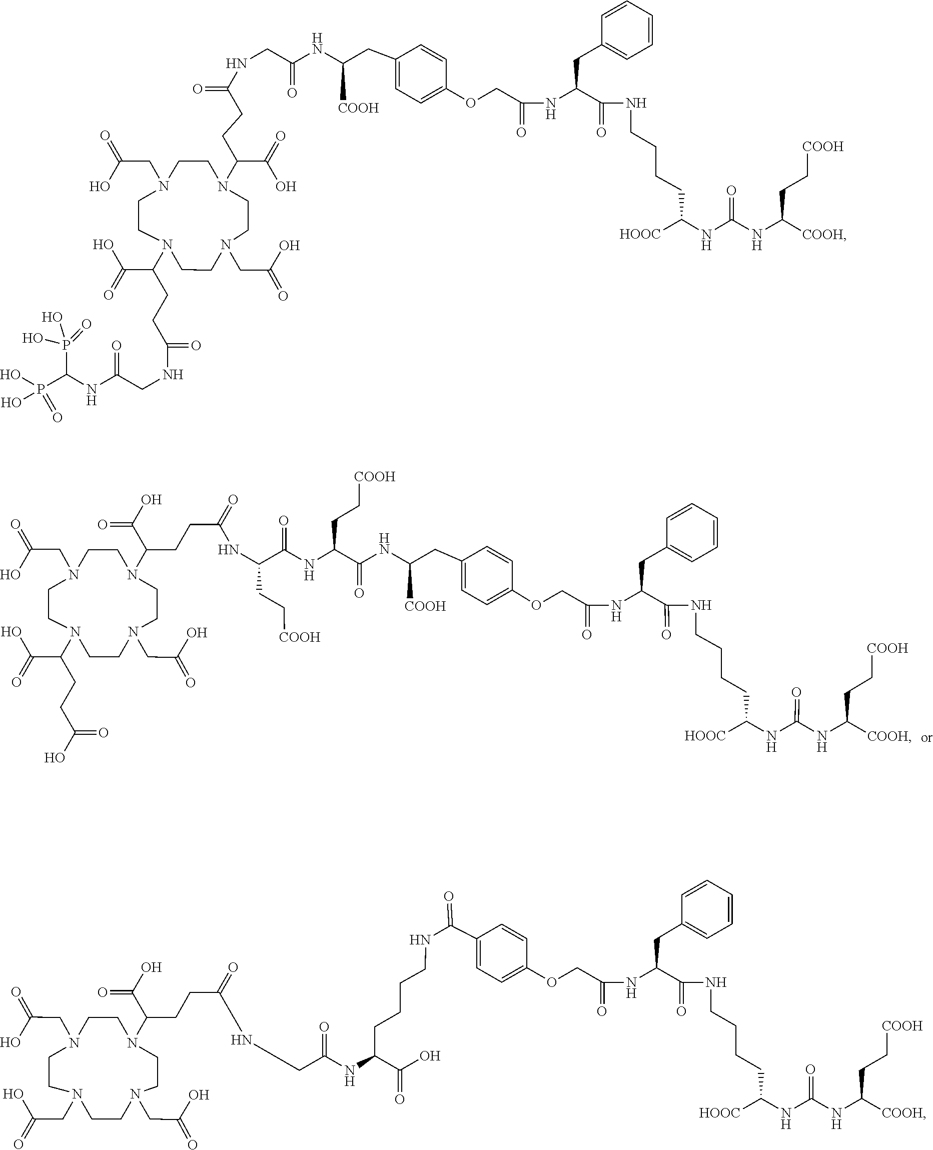
or a pharmaceutically acceptable salt thereof.

29. The compound of claim 1, having the structure: 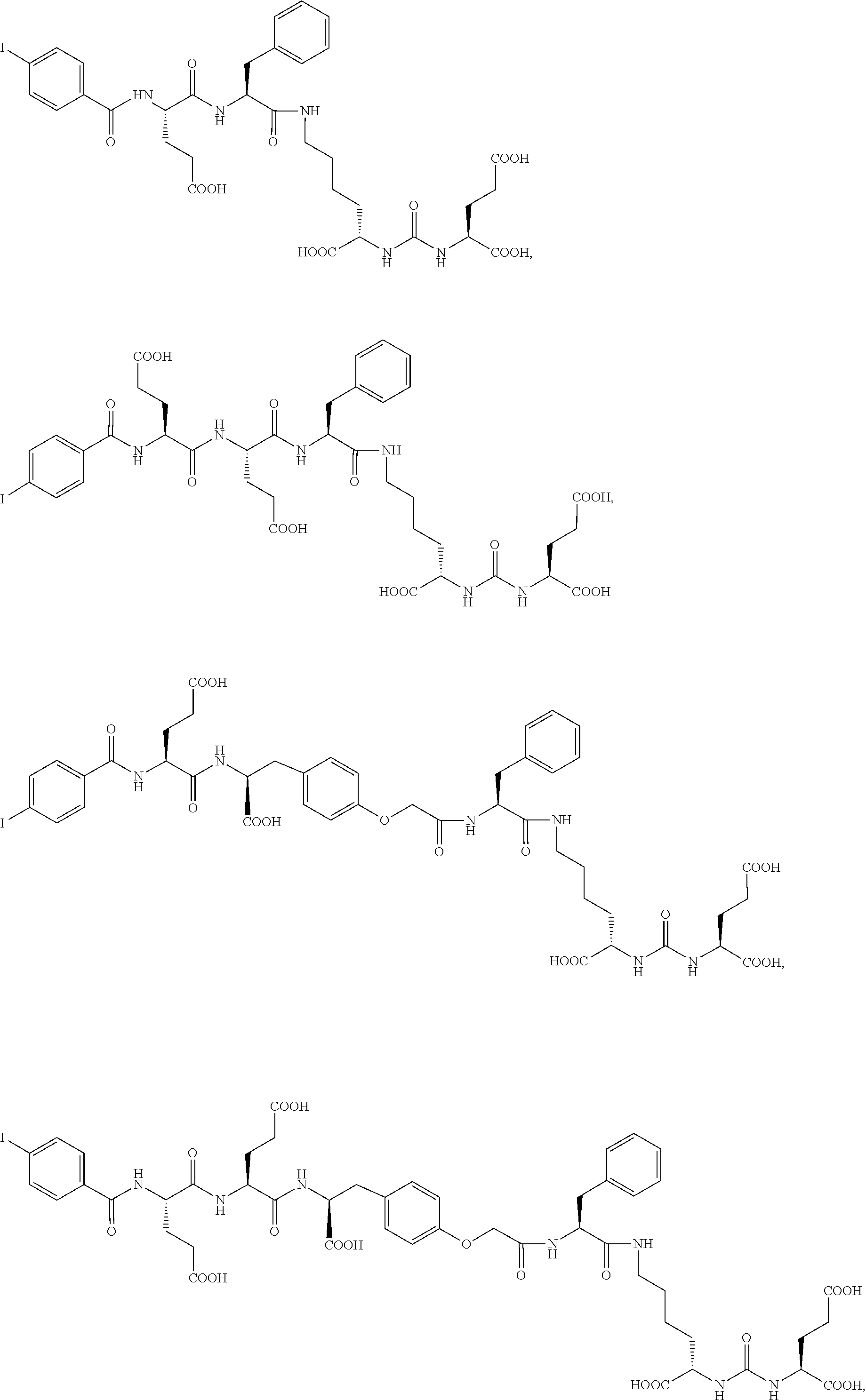
or a pharmaceutically acceptable salt thereof, wherein I is radioactive.

30. A complex comprising the compound according to any one of claims 1 to 4 and 7 to 28 and a metal M chelated to the chelating moiety of the compound, wherein M is selected from the group consisting of ²²⁵Ac, ⁴⁴Sc, ⁴⁷Sc, ^{203/212}Pb, ^{67Ga}, ⁶⁸Ga, ⁷²As, ^{99m}Tc, ¹¹¹In, ⁹⁰Y, ⁹⁷Ru, ⁶²Cu, ⁶⁴Cu, ⁵²Fe, ^{52m}Mn, ¹⁴⁰La, ¹⁷⁵Yb, ¹⁵³Sm, ¹⁶⁶Ho, ¹⁴⁹Pm, ¹⁷⁷Lu, ¹⁴²Pr, ¹⁵⁹Gd, ²¹³Bi, ⁶⁷Cu, ¹¹¹Ag, ¹⁹⁹Au, ¹⁶¹Tb, and ⁵¹Cr.

31. The complex of claim 30, having the structure: 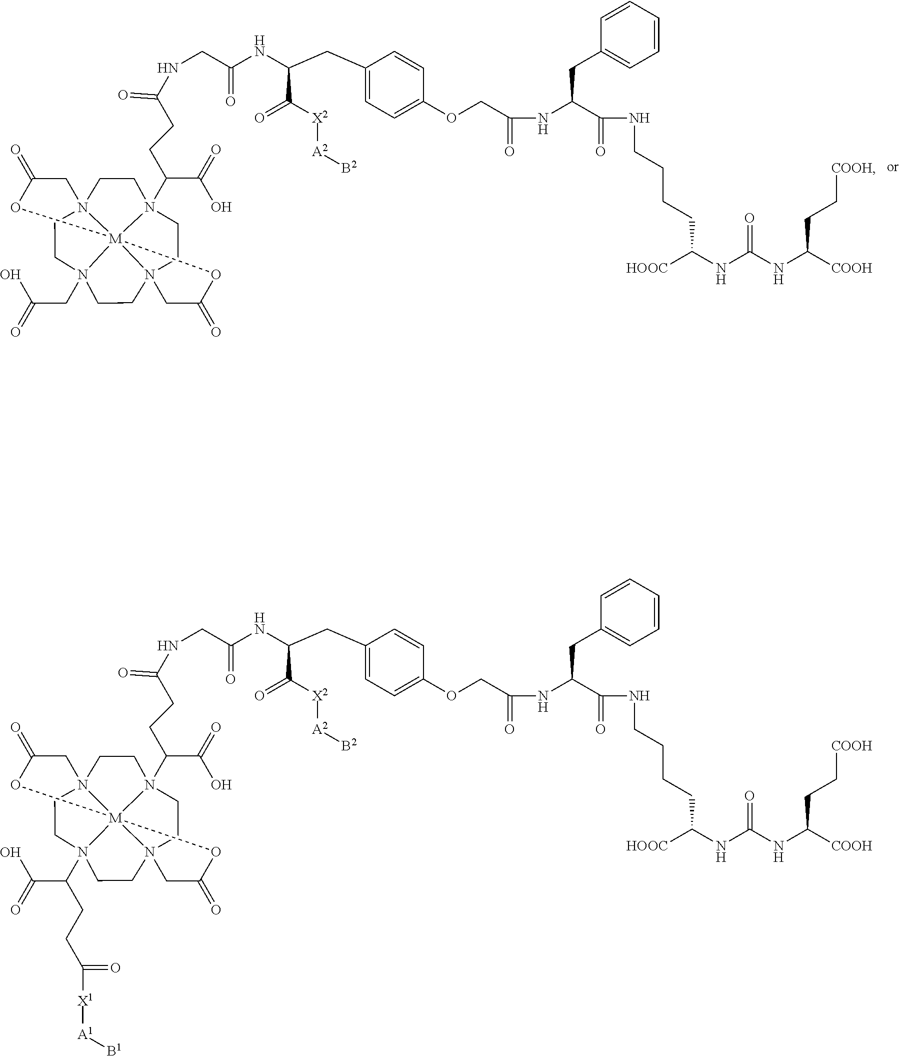
or a pharmaceutically acceptable salt thereof.

32. The complex of claim 31, or a pharmaceutically acceptable salt thereof, wherein
X¹ is O or —NH—;
X² is O or —NH—;
A¹ is a bond, —(CH₂)C(O)NH—, or —(CH₂CH₂O)₂(CH₂CH₂NH)—;
A² is a bond or —(CH₂)C(O)NH—; and
each of B¹ and B² is independently H,

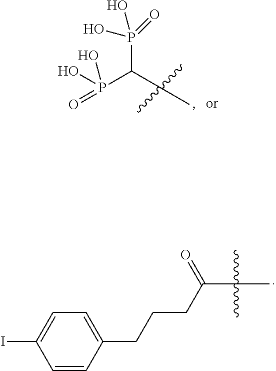 [SMILES: *C(P(=O)(O)O)P(=O)(O)O ; *C(=O)CCCc1ccc(I)cc1]

33. The complex of any one of claims 30 to 32, or a pharmaceutically acceptable salt thereof, wherein M is ⁶⁸Ga or ¹⁷⁷Lu.

34. The complex of claim 30, having the structure: 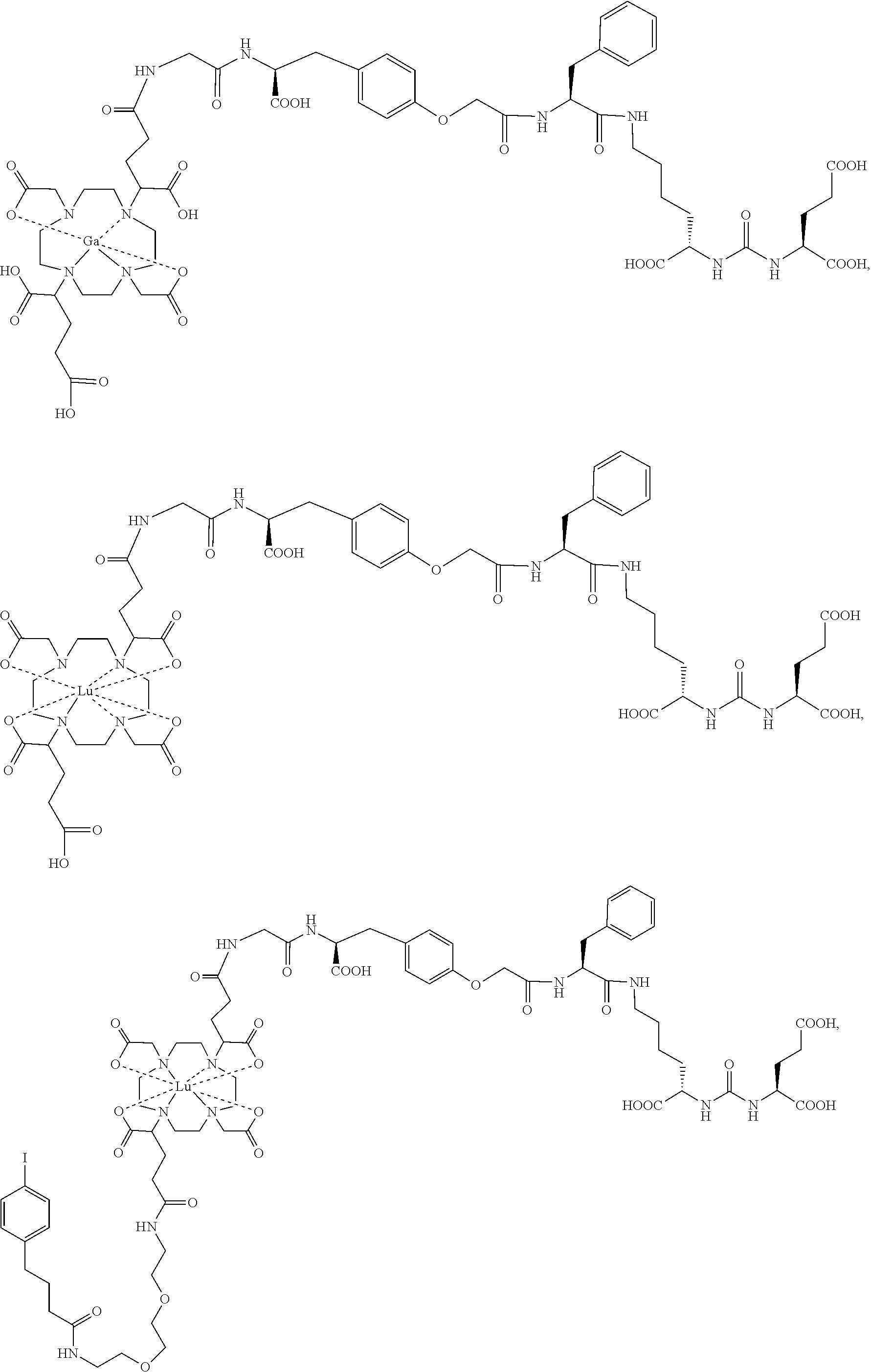 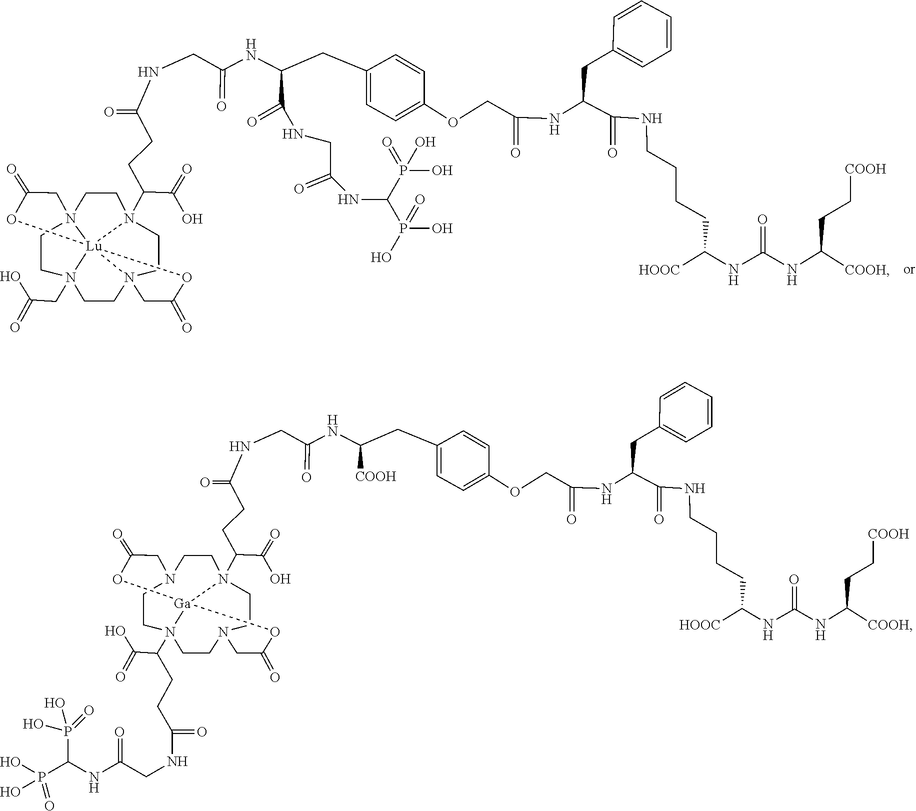
or a pharmaceutically acceptable salt thereof.

35. A pharmaceutical composition comprising a pharmaceutically acceptable carrier and the compound or complex according any one of claims 1 to 34 or a pharmaceutically acceptable salt thereof.

36. A method for imaging in a subject, comprising
administering the compound or complex of any one of claims 1, 5-14, 21-27, 29, and 30-34 to said subject; and
obtaining an image of said subject or a portion of said subject.

37. The method of claim 36, comprising obtaining an image with a device that is capable of detecting positron emission.

38. A method of in vivo imaging comprising administering an effective amount of the compound or complex according any one of claims 1, 5-14, 21-27, 29, and 30-34 to a subject, and detecting the pattern of radioactivity of the complex in said subject.

39. A method of treating one or more tumors in a subject, comprising administering an effective amount of the compound or complex according any one of claims 1, 5-14, 21-27, 29, and 30-34 to the subject.

40. A kit comprising a sterile container containing an effective amount of the compound of any one of claims 1 to 29 or a pharmaceutically acceptable salt thereof, and instructions for therapeutic use.
